(19) United States
(12) Patent Application Publication
(10) Pub. No.: US 2026/0234589 A1
(43) Pub. Date: Aug. 13, 2026

(54) INFLUENZA NEURAMINIDASE TETRAMERS

(71) Applicant: ICOSAVAX, INC., Seattle, WA (US)

(72) Inventor: Daniel ELLIS, Seattle, WA (US)

(21) Appl. No.: 19/398,620

(22) Filed: Nov. 24, 2025

Related U.S. Application Data
(63) Continuation of application No. PCT/US2024/031855, filed on May 31, 2024.
(60) Provisional application No. 63/505,446, filed on Jun. 1, 2023.
(60) Provisional application No. 63/598,726, filed on Nov. 14, 2023.

(51) Int. Cl. C12N 9/24; A61K 39/145; A61P 37/04; C07K 14/005; C07K 14/195

(52) U.S. Cl. CPC C12N 9/2402; A61K 39/145; A61P 37/04; C07K 14/005; C07K 14/195; C12Y 302/01018; C07K 2319/55; C12N 2760/16022; C12N 2760/16034

(57) ABSTRACT

The disclosure relates to compositions that include a recombinant influenza neuraminidase tetramer that includes four copies of a fusion protein which includes a DA4B tetramerization domain, optionally a linker, and an influenza NA head domain, and uses thereof. Novel linkers are provided. The disclosure further relates to a recombinant influenza neuraminidase (NA) polypeptide, comprising an influenza NA head domain comprising one or more amino acid substitutions selected from the group consisting of I99P, Y100L, T131Q, C161V, E165S, S172A, V177I, S196T, V205I, Q408M, R419V, and V453T. Further provided are compositions, methods of making, and methods of use, for example as vaccines for influenza.

### CROSS-REFERENCE TO RELATED APPLICATIONS

This application is a continuation of International Application No. PCT/US2024/031855, filed May 31, 2024, which claims priority benefit under 35 U.S.C. § 119(e) to U.S. Provisional Patent Application No. 63/505,446, filed Jun. 1, 2023, and U.S. Provisional Patent Application No. 63/598,726, filed Nov. 14, 2023, each of which is incorporated by reference herein in its entirety.

### SEQUENCE LISTING

This application contains a Sequence Listing which has been submitted in .XML format via EFS-WEB and is hereby incorporated by reference in its entirety. Said .XML copy, created on Nov. 5, 2025, is named 061291-511001WO_SeqList_ST26_v2.xml and is 630,784 bytes in size.

### BACKGROUND OF THE INVENTION

Influenza viruses are enveloped single-stranded RNA viruses that occur in three distinct antigenic types (i.e., A, B and C). Types A and B are responsible for epidemics, while type C causes mild respiratory illness. There are two major proteins on the surface of virion, hemagglutinin (HA) and neuraminidase (NA), which define distinct viral subtypes. Hemagglutinin (HA) initiates infection by binding to sialic acid residues present on surface of a host cell and performing membrane fusion.

Neuraminidase (NA) is a surface antigen on influenza viruses and is targeted by protective antibodies. Current influenza vaccines do not effectively elicit productive anti-NA antibody responses, and inclusion of recombinant NA proteins in vaccines offers potential to improve this. Neuraminidase is essential for the replication of influenza virus by cleaving terminal sialic acid residues from carbohydrate moieties on host cell membrane proteins and on viral envelope glycoprotein spikes of newly synthesized virions, which promotes release of influenza virions from infected cells and thereby dissemination of virus within the respiratory tract. It may also reduce the antiviral effect of respiratory mucus. Inhibition of neuraminidase promotes aggregation of viral particles on the surface of infected cells and interrupts the replicative cycle of the virus.

Tetramerization domains have been fused in the head domain of influenza NA to generate soluble tetramers intended as vaccine antigens and reagents. In one case, the influenza NA was expressed as a soluble tetramer using an N-terminal fusion with the tetramerization domain tetrabrachion. Other tetramerization domains tested have included VASP and MPP. To date, such constructs have been proposed as vaccine antigens. However, none have progressed to approved products for prevention or treatment of influenza.

Accordingly, there remains a long-felt and unmet need in the art for recombinant influenza neuraminidase (NA) tetramers, which may be used as vaccine antigens or as reagents.

### SUMMARY

The disclosure relates to compositions that include a recombinant influenza neuraminidase tetramer that includes four copies of a fusion protein which includes a DA4B tetramerization domain, optionally a linker, and an influenza NA head domain, and uses thereof. Novel linkers are provided. The disclosure further relates to a recombinant influenza neuraminidase (NA) polypeptide, comprising an influenza NA head domain comprising one or more amino acid substitutions selected from the group consisting of I99P, Y100L, T131Q, C161V, E165S, S172A, V177I, S196T, V205I, Q408M, R419V, and V453T. Further provided are compositions, methods of making, and methods of use, for example as vaccines for influenza.

In one aspect, the disclosure provides a recombinant influenza neuraminidase (NA) tetramer including four copies of a fusion protein. The fusion protein including, in N- to C-terminal order, a DA4B tetramerization domain, optionally a linker, and an influenza NA head domain, wherein the DA4B tetramerization domain comprises, consists essentially of, or consists of a DA4B polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5.

In some embodiments, the tetramer of the DA4B tetramerization domain comprises between 30 and 60 amino residues. In some embodiments, the tetramer of the DA4B polypeptide sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5. In some embodiments, the tetramer of the DA4B polypeptide sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1. In some embodiments, the tetramer of the DA4B polypeptide sequence is SEQ ID NO: 5. In some embodiments, the tetramer of the DA4B polypeptide sequence is SEQ ID NO: 1.

In some embodiments, the linker is a polypeptide bond. In some embodiments, the linker is SG, GGSGGSGG (SEQ ID NO: 11), ATPRGG (SEQ ID NO: 12), ATPRGSGG (SEQ ID NO: 13), or GGATPRGSGG (SEQ ID NO: 14).

In some embodiments, the tetramer of the influenza NA head domain comprises an NA polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15-31 and 423. In some embodiments, the tetramer of the influenza NA head domain is a head domain of a subtype N1 influenza. In some embodiments, the tetramer of the influenza NA head domain includes three or more amino acid substitutions of I99P, Y100L, T131Q, C161V, E165S, S172A, V177I, S196T, V205I, Q408M, R419V, and V453T, numbered relative SEQ ID NO: 15.

In some embodiments, the tetramer of the influenza NA head domain comprises one of the combinations of amino acid substitutions listed below, numbered relative SEQ ID NO: 15:
a. I99P, C161V, E165S, S172A, S196T,
b. I99P, T131Q, C161V, E165S, S172A, S196T,
c. I99P, T131Q, C161V, E165S, S172A, V177I, S196T,
d. I99P, T131Q, C161V, E165S, S172A, S196T, V453T,
e. I99P, T131Q, C161V, E165S, S172A, V177I, S196T, V205I,
f. I99P, Y100L, T131Q, C161V, E165S, S172A, V177I, S196T, V205I, Q408M, R419V,
g. I99P, C161V, E165S, S172A, V177I, S196T,
h. I99P, C161V, S172A, S196T, or
i. I99P, C161V, S172A, V177I, S196T.

In some embodiments, the disclosure provides:
a. the NA head domains are in a closed conformation,
b. the tetramer has increased thermal stability in the presence of chelating agent compared to a tetramer with tetrabrachion tetramerization domain,
c. the tetramer has increased interaction to neuraminidase inhibitor compared to a tetramer with tetrabrachion tetramerization domain, and/or
d. the tetramer is expressed in recombinant cell culture at increased yield compared to a tetramer with a tetrabrachion tetramerization domain.

In some embodiments, the fusion protein comprises the tetramer described herein. In some embodiments, the fusion protein comprises, consists essentially of, or consists of a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 32-422.

In another aspect, the disclosure provides a polynucleotide encoding the fusion protein comprised by the tetramer described herein, optionally the polynucleotide is an mRNA.

In another aspect, the disclosure provides a delivery vehicle comprising the polynucleotide described herein, optionally the delivery vehicle is a lipid nanoparticle.

In another aspect, the disclosure provides a pharmaceutical composition, comprising the compositions described herein and a pharmaceutically acceptable carrier.

In another aspect, the disclosure provides a method of immunizing a subject in need thereof against infection by influenza, the method comprising administering to the subject an effective amount of a composition of the disclosure.

In another aspect, the disclosure provides a method of treating and/or preventing influenza in a subject in need thereof, the method comprising administering to the subject an effective amount of a composition of the disclosure.

In some embodiments, the method generates an antibody response to the tetramer equivalent to or greater than the antibody response to administration of a tetramer with a tetrabrachion tetramerization domain.

In another aspect, the disclosure provides a kit including a composition of the disclosure and instructions for use.

In another aspect, the disclosure provides a host cell comprising a polynucleotide described herein.

In another aspect, the disclosure describes a method of producing a recombinant influenza neuraminidase (NA) tetramer, comprising culturing the host cell of the disclosure in cell culture media, and harvesting the recombinant influenza neuraminidase (NA) tetramer from the cell culture media or the host cell.

In some embodiments, the method produces an increased yield of tetramer compared to production of a tetramer with a tetrabrachion tetramerization domain.

In another aspect, the disclosure provides a fusion protein comprising a DA4B tetramerization domain and a heterologous polypeptide, wherein the DA4B tetramerization domain comprises, consists essentially of, or consists of a DA4B polypeptide sequence at least 75%, at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOs: 1-5.

In another aspect, the disclosure provides a recombinant influenza neuraminidase (NA) polypeptide, comprising an influenza NA head domain comprising one or more amino acid substitutions selected from the group consisting of I99P, Y100L, T131Q, C161V, E165S, S172A, V177I, S196T, V205I, Q408M, R419V, and V453T, numbered relative SEQ ID NO: 15.

In some embodiments of the polypeptide of the present application, the influenza NA head domain comprises three or more amino acid substitutions selected from the group consisting of I99P, Y100L, T131Q, C161V, E165S, S172A, V177I, S196T, V205I, Q408M, R419V, and V453T, numbered relative SEQ ID NO: 15.

In some embodiments of the polypeptide of the present application, the influenza NA head domain comprises the amino acid substitutions, numbered relative SEQ ID NO: 15, selected from:
a. I99P, C161V, E165S, S172A, S196T;
b. I99P, T131Q, C161V, E165S, S172A, S196T;
c. I99P, T131Q, C161V, E165S, S172A, V177I, S196T;
d. I99P, T131Q, C161V, E165S, S172A, S196T, V453T;
e. I99P, T131Q, C161V, E165S, S172A, V177I, S196T, V205I;
f. I99P, Y100L, T131Q, C161V, E165S, S172A, V177I, S196T, V205I, Q408M, R419V;
g. I99P, C161V, E165S, S172A, V177I, S196T;
h. I99P, C161V, S172A, S196T; or
i. I99P, C161V, S172A, V177I, S196T.

In some embodiments of the polypeptide of the present application, the influenza NA head domain comprises an NA polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15-31 and 423.

In some embodiments of the polypeptide of the present application, the influenza NA head domain is a head domain of a subtype N1 influenza.

In some embodiments of the polypeptide of the present application,
a. the NA head domains are in a closed conformation;
b. the tetramer has increased thermal stability in the presence of chelating agent compared to a tetramer with tetrabrachion tetramerization domain;
c. the tetramer has increased interaction to neuraminidase inhibitor compared to a tetramer with tetrabrachion tetramerization domain; and/or
d. the tetramer is expressed in recombinant cell culture at increased yield compared to a tetramer with a tetrabrachion tetramerization domain.

In another aspect, the disclosure provides a fusion protein, comprising, in N- to C-terminal order, a tetramerization domain and an influenza NA polypeptide disclosed herein.

In another aspect, the disclosure provides a recombinant influenza neuraminidase (NA) tetramer, comprising four copies of a fusion protein comprising, in N- to C-terminal order, a tetramerization domain and an influenza NA polypeptide disclosed herein.

In another aspect, the disclosure provides a polynucleotide encoding the fusion protein disclosed herein, optionally wherein said polynucleotide is an mRNA.

In another aspect, the disclosure provides a delivery vehicle comprising the polynucleotide disclosed herein, optionally wherein said delivery vehicle is a lipid nanoparticle.

In another aspect, the disclosure provides a pharmaceutical composition, comprising the composition disclosed herein and a pharmaceutically acceptable carrier.

In another aspect, the disclosure provides a method of immunizing a subject in need thereof against infection by influenza, the method comprising administering to the subject an effective amount of a composition disclosed herein.

In another aspect, the disclosure provides a method of treating and/or preventing influenza in a subject in need thereof, the method comprising administering to the subject an effective amount of a composition disclosed herein.

In some embodiments, the method generates an antibody response to the tetramer equivalent to or greater than the antibody response to administration of at least as effectively as administering a tetramer with a tetrabrachion tetramerization domain.

In another aspect, the disclosure provides a kit comprising a composition disclosed herein and instructions for use.

In another aspect, the disclosure provides a host cell comprising the polynucleotide disclosed herein.

In another aspect, the disclosure provides a method of producing a recombinant influenza neuraminidase (NA) tetramer, comprising culturing the host cell disclosed herein in cell culture media, and harvesting the recombinant influenza neuraminidase (NA) tetramer from the cell culture media or the host cell.

In some embodiments, the method produces an increased yield of tetramer compared to production of a tetramer with a tetrabrachion tetramerization domain.

In another aspect, the disclosure provides a polypeptide, comprising the polypeptide sequence ATPRGG (SEQ ID NO: 12), ATPRGSGG (SEQ ID NO: 13), or GGATPRGSGG (SEQ ID NO: 14).

In another aspect, the disclosure provides a recombinant influenza neuraminidase (NA) tetramer, comprising four copies of a fusion protein, said fusion protein comprising, in N- to C-terminal order, a tetramerization domain, a linker, and an influenza NA head domain, wherein the linker is or comprises the polypeptide sequence ATPRGG (SEQ ID NO: 12), ATPRGSGG (SEQ ID NO: 13), or GGATPRGSGG (SEQ ID NO: 14).

In another aspect, the disclosure describes a composition, method, or use as described herein.

Any tetramer, fusion protein, polynucleotide, delivery vehicle, VLP, composition or method disclosed herein is applicable to any herein-disclosed tetramer, fusion protein, polynucleotide, delivery vehicle, composition or method. In other words, any aspect or embodiment described herein can be combined with any other aspect or embodiment as disclosed herein.

### BRIEF DESCRIPTION OF THE DRAWINGS

FIGS. 1A-1E show structural modeling of DA4B. FIG. 1A shows Sequence alignment of 4B, DA4B, and source sequences. Added terminal residues are in parentheses. Buried hydrophobic residues in PDB 1FE6 are marked with asterisks, and the T51S mutation in DA4B is marked with a caret. FIG. 1B shows a model of the tetrabrachion tetramerization domain (“TB”) from PDB 1FE6. FIG. 1C shows a Rosetta homology model of DA4B generated from PDB 1FE6.

FIG. 1D shows a ColabFold model of DA4B. One chain each in B-D shows sidechains, with buried hydrophobic residues shown as spheres and exposed residues showed as lines. FIG. 1E shows representation of NA designs studied.

FIG. 2 shows a western blot of initial designs for N1, N2 and B soluble tetramers using TB, DA4B, modified versions of DA4B or VASP tetramerization domains. These data show that N1 designs using DA4B provide higher yields than equivalent designs that use TB.

FIGS. 3A-3H show SEC (A-D) and catalytic activity (E-H) of representative equivalent designs which compare DA4B and TB when genetically fused to N1 (A, E), N2 (B, F), By (C, G), and By antigens (D, H). SEC in A and B was performed on a Superdex 200 Increase 10/300 gl column, while SEC in C and D was performed on a Superose 6 Increase 10/300 gl column. SEC was performed on IMAC eluates produced from cell cultures of the same volume for constructs within each panel. For catalytic activity, each symbol marks a measurement at a particular dilution of the NA sample, while lines connect measurements from the same sample. FIG. 3A shows SEC of L2_70-N1-MI15 genetically fused with either DA4B or TB. FIG. 3B shows catalytic activity of L2_70-N1-MI15 genetically fused with either DA4B or TB. FIG. 3C shows SEC of L8_83-N2-DA21 genetically fused with either DA4B or TB. FIG. 3D shows catalytic activity of L8_83-N2-DA21 genetically fused with either DA4B or TB. FIG. 3E shows SEC of L8_75-Bv-CO17 genetically fused with either DA4B or TB. FIG. 3F shows catalytic activity of SEC of L8_75-Bv-CO17 genetically fused with either DA4B or TB. FIG. 3G shows SEC of L8_75-Bv-PH13 genetically fused with either DA4B or TB. FIG. 3H shows catalytic activity of SEC of L8_75-Bv-PH13 genetically fused with either DA4B or TB.

FIGS. 4A-4E show catalytic activity of N1, N2, Bv, and By NA antigens that alter the connection between DA4B and the NA head. Each symbol marks a measurement at a particular dilution of the NA sample, while lines connect measurements from the same sample. FIG. 4A shows that the use of the L2 linker and an N1 NA start point of 65 leads to lower catalytic activity than at start point at 70. FIG. 4B shows that the connection between DA4B and the N1 NA head can be shortened to 6 residues while maintaining high catalytic activity. FIG. 4C shows that the use of the L2 linker and an N2 NA start point of 65 leads to lower catalytic activity than other shorter connections. FIG. 4D shows that no difference in catalytic activity were observed within the By NA antigens studied. FIG. 4E shows that no differences in catalytic activity were observed within the By NA antigens studied.

FIG. 5 shows catalytic activity of select N1 soluble tetramers. Each symbol marks a measurement at a particular dilution of the NA sample, while lines connect measurements from the same sample. These data show that many combinations of stabilizing mutations can negatively impact catalytic activity.

FIGS. 6A-6D show enzyme-linked lectin assay (ELLA) titers elicited by N1 or N2 soluble tetramers in mice after one (day 21) and two (day 35) doses. Each dose consisted of lug of antigen adjuvanted with Addavax™. The “Pre-Bleed Pool Day 0” group consists of pooled serum from animals prior to immunization. FIG. 6A shows evaluation of strain-matched ELLA titers for N2 soluble tetramers, showing similar immunogenicity between all vaccine groups at both timepoints. FIG. 6B shows evaluation of strain-matched ELLA titers for one set of N1 soluble tetramers, showing no significant differences between vaccine groups on day 21, and one significant difference on day 35 with DA4B_L2_70-N1-MI15-6mutB higher than TB_L2_70-N1-MI15. FIG. 6C shows evaluation of strain-matched ELLA titers for a different set of N1 soluble tetramers, showing no significant differences between vaccine groups on day 21, and two significant difference on day 35 with DA4B_L2_70-N1-MI15 and DA4B_L2_70-N1-MI15-7mutA both higher than DA4B_L2_70-N1-MI15-11mut. FIG. 6D shows evaluation of strain-matched ELLA titers for TB_L8_75-Bv-CO17 and DA4B_L8_75-Bv-CO17, showing no significant differences between vaccine groups on days 21 and 35.

FIGS. 7A-7B show catalytic activity of select N1 soluble tetramers. FIG. 7A shows catalytic activity of neuraminidase mutants in A/Wisconsin/588/2019 strain. FIG. 7B shows catalytic activity of neuraminidase mutants in A/Vietnam/1203/2004 strain.

FIG. 8 shows western blot of supernatants produced at small-scale for variants of DA4B_L2_70-N1-MI15 with N-terminally shortened tetramerization domains. These data show that N-terminal deletions of up to 15 residues could be performed while maintaining similar yields. Binding to oseltamivir for these samples is provided in Table 18.

FIG. 9 shows SEC spectrum of IMAC eluate from DA4B-2glyc_L10_83-N2-DA21, which showed significant smaller species that were not consistent with tetramer formation. Performed on a Superdex 200 Increase 10/300 gl column.

FIG. 10A shows a graph depicting a biolayer interferometry (BLI) assay of N1 soluble tetramer antigens binding to oseltamivir (left panel) or CD6 mAb (right panel) in the presence of added CaCl₂). Selected shift values from these data (100 seconds into association phase) are also shown in Tables 7 and 9.

FIG. 10B shows a SYPRO® thermal denaturation fluorescence assay of stabilized N1 soluble tetramer antigens from various influenza strains in the presence of added CaCl₂ (left: A/Michigan/45/2015; middle: A/Wisconsin/588/2019; and right: A/Vietnam/1203/2004). Melting temperatures are marked by vertical lines, and are labelled above each vertical line. Melting temperatures from these data are also shown in Tables 13 and 16

FIG. 11A is a diagram of study reagents and study design used to test co-formulations of an HA VLP nanostructure and an N2 soluble tetramer (DA4B_L8_83-N2-DA21).

FIG. 11B shows measurement of Hemagglutination inhibition titers (HAI) assay against H1N1, H3N2, and By viruses from mouse serum dosed with a combination of HA VLP nanostructure and soluble NA tetramer at Day 35. “HA” and “NA” labels respectively refer to the HA VLP nanostructure and the N2 soluble tetramer (DA4B_L8_83-N2-DA21). HA antigens from each virus were matched to the H1, H3 and By HA antigens used in the HA nanostructure.

FIG. 11C shows measurement of anti-N2 (A/Darwin/6/2021) response by ELLA assay from mouse serum dosed with combinations of HA (“HA”) nanostructure and/or soluble NA tetramer (“NA”) at Day 35.

### DETAILED DESCRIPTION

### Definitions

Reference is made herein to particular features (including method steps). It is to be understood that the disclosure in this specification includes all possible combinations of such particular features. For example, where a particular feature is disclosed in the context of a particular aspect or embodiment, or a particular claim, that feature can also be used, to the extent possible, in combination with and/or in the context of other particular aspects and embodiments.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The practice of the technology will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. It is to be understood that this disclosure is not limited to the particular methodology, protocols, and reagents described, as these may vary, depending upon the context in which they are used by those of skill in the art.

All publications and patents mentioned herein are hereby incorporated by reference in their entirety as if each individual publication or patent was specifically and individually indicated to be incorporated by reference. In case of conflict, the present application, including any definitions herein, controls. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described. Specifically, features described in one section may be combined with features in any other section of the description.

While illustrative embodiments are described and depicted, it will be appreciated that various changes can be made to these illustrative embodiments without departing from the spirit and scope of the invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole.

The singular forms “a”, “an”, and “the” are intended to include the plural forms as well, unless the context clearly indicates otherwise.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10%, +/−5%, +/−3%, or +/−1% of the specified value.

The term “at least” followed by a number is used herein to denote the start of a range beginning with that number (which may be a range having an upper limit or no upper limit, depending on the variable being defined). For example, “at least 1” means 1 or more than 1.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number)” this means a range whose lower limit is the first number and whose upper limit is the second number. For example, 25 to 100 mm means a range whose lower limit is 25 mm, and whose upper limit is 100 mm.

Throughout this specification, unless the context requires otherwise, the words “comprise”, “comprises” and “comprising” will be understood to imply the inclusion of a stated step or element or group of steps or elements but not the exclusion of any other step or element or group of steps or elements. For example, a composition “comprising” (or “which comprises”) components A, B, and C can consist of (i.e., contain only) components A, B, and C, or can contain not only components A, B, and C but also one or more other components.

As used herein, the term “consisting of” refers to including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory and other elements may be included if they do not materially affect the basic and novel characteristic(s) of the invention.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The terms “identical,” “identity,” “percent identity,” “sequence identity,” or “percent sequence identity,” in the context of two or more nucleic acid or polypeptide sequences, refers to two or more sequences or subsequences that are the same or have a specified percentage of amino acid residues or nucleotides that are the same, when compared and aligned for maximum correspondence. Methods of alignment of sequences for comparison are well known in the art. Once aligned, the number of matches is determined by counting the number of positions where an identical nucleotide or amino acid residue is present in both sequences. The percent sequence identity is determined by dividing the number of matches in the alignment by the length of the reference sequence, followed by multiplying the resulting value by 100. For example, a peptide sequence that has 1166 matches when aligned with a test sequence having 1554 amino acids is 75.0 percent identical to the test sequence (1166÷1554*100=75.0). As the terms are used herein, gaps in the alignment do not decrease the percent sequence identity.

Unless otherwise specified, optimal alignment of sequences for comparison is conducted by the global alignment algorithm of Needleman and Wunsch, Mol. Biol. 48:443 (1970) as implemented by EMBOSS Needle (see, the World Wide Web at ebi.ac.uk/Tools/psa/emboss_needle/) (Madeira et al. Nucleic Acids Res. 50(W1):W276-W279 (2022)). In embodiments, other alignment methods may be used, including without limitation those described in Devereux, et al, Nucleic Acids Res. 12:387-95 (1984); Atschul et al. J. Mo. Biol. 215:403-10 (1990) (BLAST); Carrillo and Lipman Siam J. Appl. Math. 48(5) (1988); Computational Molecular Biology (Lesk, A M, ed., 1989); Biocomputing Informatics and Genome Projects, (Smith, D W, ed., 1993); Computer Analysis of Sequence Data, Part I, (Griffin and Griffin, eds., 1994); Sequence Analysis in Molecular Biology (von Heinje, 2012); Sequence Analysis Primer (Gribskov and Devereux, J., eds. 1993). In embodiments, sequence identity is calculated using the implementation of the Needleman-Wunsch algorithm provided by the National Library of Medicine (See, the World Wide Web at blast.ncbi.nlm.nih.gov/Blast.cgi?PAGE_TYPE=BlastSearch&BLAST_SPEC=GlobalAln).

For example, sequence identity can be determined by standard methods that are commonly used to compare the similarity of two polypeptide or two polynucleotide sequences. Using a computer program such as EMBOSS Needle or BLAST, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) that can be used in conjunction with the computer program.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “relative to” or “numbered with reference to” or “corresponding to,” when used in the context of the numbering of a given sequence, refers to the numbering of the residues of the given sequence when aligned to a specified reference sequence, such that each position in the numbered sequence is associated with a number corresponding to the number of the counterpart position in the reference sequence.

As used herein an “influenza virus”, a member of the family Orthomyxoviridae includes influenza virus type A, influenza virus type B and influenza virus type C and all influenza virus subtypes. “Influenza type” as used herein refers to influenza type A, type B or type C. “Influenza strain” as used herein refers to different influenza A viruses, for example H1N1, H1N2, H1N7, H2N2, H3N2, H3N8, H4N8, H5N1, H5N2, H5N9, H6N2, H6N5, H7N2, H7N3, H7N7, H8N4, H9N2, H10N7, H11N6, H12N5, or H13N6. “Neuraminidase” or “NA” as used herein refers to a neuraminidase protein of influenza virus and may be of any influenza type, A, B or C, and any serotype, for example, N1, N2, N3, N4, N5, N6, N7, N8, N9, N10, or N11 including subtypes belonging to the same serotype but with a small difference as a result of for example antigenic drift. As used herein, the term “neuraminidase” refers to immunological cross reactivity with the native neuraminidase of one or more strains of influenza virus. The literature is replete with examples of such known neuraminidases (e.g., in GenBank, in publications from the CDC, etc.). Again, the literature is replete with examples of such known neuraminidase molecules.

The term “head domain” or “NA head domain” as used herein refers to the folded domain of the native influenza NA present on the surface of the virion. It has neuraminidase (also known as sialidase) activity and cleavage of sialic acid promotes release of virions from the cell. The head domain forms intratetramer contacts in the so-called “closed” formation of the influenza NA. In recombinant polypeptides that include a head domain, as described herein, the head domain may be in the closed or open conformation. Head domains suitable for use as vaccines maintain at least one antigenic epitope. The sialidase activity may serve to identify head domains in the closed conformation. However, head domains may lack sialidase activity and still have utility as vaccine antigens. In some cases, the “head domain” or “NA head domain” comprises the beginning of the globular part of NA, and does not include the stalk.

The term “NA antigen” includes about the entirety of the NA molecule, including the tetramerization domain. In other words, this term could be considered as an “NA immunogen”.

The term “antigen fragment”, e.g., “NA antigen fragment” includes the starting and ending residues of the region derived from the pathogen. Because some of the part derived from the pathogen is also the NA stalk (and not just the NA head domain), this may be different from a NA head domain.

The term “linker” refers to a peptide bond or polypeptide that chemically joins the C terminus of a first polypeptide segment to the N terminus of a second polypeptide segment, thereby forming a fusion protein containing the two polypeptide segments.

The term “tetramerization domain” as used herein is defined as a domain that mediates the formation of a tetramer out of four monomeric proteins or parts thereof.

The terms “polypeptide”, “peptide” and “protein” are used interchangeably herein to refer to a polymer of amino acid residues, wherein the polymer may be conjugated to a moiety that does not consist of amino acids, such as a glycan or other post-translational modification, or a non-natural chemical moiety. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single polymer molecule.

The term “subtype” or “serotype” as used herein, interchangeably, and in reference to influenza virus, means genetic variants of an influenza virus such that one subtype is recognized by an immune system apart from a different subtype.

The term “substitution” refers to replacing a single amino acid in a sequence with another amino acid residue. The standard form of abbreviations for amino acid substitution are used. For example, V94R refers to substitution of valine (V) in a reference sequence with arginine (R). The abbreviation Arg94 refers to any sequence in which the 94th residue, relative to a reference sequence, is arginine (Arg).

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The term “antigenic variant” refers to a variant that has one or more epitopes in common with a reference polypeptide and/or generates the same or similar immune response when administered to a subject as a reference polypeptide.

The term “antigenic fragment” refers to any fragment of a protein that generates an immune response (humoral or T cell response) to the protein in vivo. The antigenic fragment may be a linear epitope, discontinuous epitope, or a conformation epitope (e.g., a folded domain). The antigenic fragment may preserve the secondary, tertiary, and/or quaternary structure of the full-length protein.

As used herein, “chelating agents” or “chelators” refers to chemicals that bind certain metal ions, inactivating the ions so that they cannot react with other elements forming chelates. In the present context the term “chelating agents” comprises chelants, chelating agent, or chelating agents that form water-soluble complexes with metal ions such as calcium and magnesium.

An “inhibitor” refers to a compound (e.g., compounds described herein) that reduces activity when compared to a control, such as absence of the compound or a compound with known inactivity.

The term “recombinant” indicates that the material (e.g., a nucleic acid or protein) has been artificially or synthetically (non-naturally) altered by human intervention. The alteration can be performed on the material within, or removed from, its natural environment or state. Specifically, e.g., an influenza virus is recombinant when it is produced by the expression of a recombinant nucleic acid. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant influenza virus, is produced by the expression of a recombinant nucleic acid. A “fusion protein” or “tetramer” as described herein is recombinant and do not occur in nature.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

As used herein, the term “messenger RNA” or “mRNA” refer to a polynucleotide that encodes at least one polypeptide. mRNA as used herein encompasses both modified and unmodified RNA. mRNA may contain one or more reading frames or regions.

The term “lipid nanoparticle” or “LNP” refers to particles formed by one or more lipid components typically used as carriers for nucleic acid, protein, or oligonucleotide delivery to target cells and often having at least one dimension on the order of nanometers (e.g., 1-1,000 nm). Generally, lipid nanoparticle compositions for delivery are composed of synthetic ionizable or cationic lipids, phospholipids, cholesterol, and a polyethylene glycol (PEG) lipid. However, these compositions may also include other lipids. In embodiments, lipid nanoparticles can include an ionizable lipid (sometimes termed a “cationic lipid” in the art), a helper lipid, a sterol, and a polymer-conjugated lipid. Without being bound by theory, LNPs protect payloads (e.g., mRNA) by encapsulating the payloads and then delivering them to cells in response to the pH decrease that occurs after internalization of the LNP by target cells.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, intraperitoneal, intratumoral, or subcutaneous route). Administration of a nanoparticle composition to a mammal or mammalian cell may involve contacting one or more cells with the nanoparticle composition.

The term “pharmaceutically acceptable excipients” and “pharmaceutically acceptable carrier” refer to a substance that aids the administration of an active agent to and absorption by a subject and can be included in the compositions of the present disclosure without causing a significant adverse toxicological effect on the patient and can mean excipients approved by a regulatory agency of the Federal or a state government or listed in the U.S. Pharmacopeia or other generally recognized pharmacopeia for use in animals, and more particularly in humans. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The terms “immunization” and “immunizing” refer to administering a composition to a subject in an amount sufficient to elicit, after one or more administering steps, a desired immune response. Immunization may comprise between one and ten, or more administrations (e.g., injections) of the composition, such as 1, 2, 3, 4, 5, 6, 7, 8, 9, 10, or more administrations. The first administration may elicit no detectable immune response as generally each subsequent administration will boost the immune response generated by prior administrations. The term “immunizing” as used herein includes post-exposure prophylaxis.

The terms “subject” or “patient” refers to a living organism to which a composition as described herein may be administered. The subject may be suffering from or be at risk for a disease or condition that can be treated by administration of a vaccine or pharmaceutical composition as provided herein. Non-limiting examples of subjects include humans, other mammals, bovines, rats, mice, dogs, monkeys, goat, sheep, cows, deer, and other non-mammalian animals. In some embodiments, the subject is human.

The term “infection” or “infectious disease” refers to a disease or condition that can be caused by organisms such as a bacterium, virus, fungi or any other pathogenic microbial agents. In embodiments, the infectious disease is caused by a pathogenic bacteria. In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection.

The term “viral infection” or “viral disease” refers to a disease or condition that is caused by a virus. Non-limiting examples of viral infections include hepatic viral diseases (e.g., hepatitis A, B, C, D, E), herpes virus infection (e.g., HSV-1, HSV-2, herpes zoster), flavivirus infection, Zika virus infection, cytomegalovirus infection, a respiratory viral infection (e.g., adenovirus infection, influenza, severe acute respiratory syndrome, coronavirus infection (e.g., SARS-CoV-1, SARS-CoV-2, MERS-CoV, COVID-19, MERS)), a gastrointestinal viral infection (e.g., norovirus infection, rotavirus infection, astrovirus infection), an exanthematous viral infection (e.g., measles, shingles, smallpox, rubella), viral hemorrhagic disease (e.g., Ebola, Lassa fever, dengue fever, yellow fever), a neurologic viral infection (e.g., West Nile viral infection, polio, viral meningitis, viral encephalitis, Japanese encephalitis, rabies), and human papilloma viral infection.

The term “administering” refers to providing a composition to a subject in a manner that permits the composition to have its intended effect. Administration for vaccination or post-exposure prophylaxis may be performed by intramuscular injection, intravenous injection, intraperitoneal injection, or any other suitable route.

“Treating” or “treatment” as used herein (and as well-understood in the art) broadly refers to any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

The term “vaccine” refers to a composition that can provide active acquired immunity to a particular disease (e.g., Flu) or a pathogen (e.g., influenza virus). A vaccine typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by a pathogen) or therapeutic (e.g., treating an infection in a subject in need thereof). The administration of vaccines is referred to as vaccination. In some examples, a vaccine composition can provide antigenic molecules or a nucleic acid, e.g. mRNA that encodes antigenic molecules (e.g. peptides) to a subject. The antigenic molecule or nucleic acid that is delivered via the vaccine composition in the subject can be expressed into antigenic molecules and allow the subject to acquire immunity against the antigenic molecules. In the context of the vaccination against infectious disease, the vaccine composition can provide antigenic molecules or mRNA encoding antigenic molecules that are associated with a certain pathogen, e.g., one or more peptides that are known to be expressed in the pathogen (e.g., pathogenic bacterium or virus).

The term “adjuvant” refers to a pharmaceutically acceptable substance that enhances the immune response to an antigen when co-administered with the antigen or administered before, during, or after administration of the antigen to a subject.

As used herein, the term “prevent” refers to a decrease in the occurrence of disease symptoms in a patient. The prevention may be complete (no detectable symptoms) or partial, such that fewer symptoms are observed than would likely occur absent treatment.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “host cell” refers to any cell capable of use in expression of a recombinant polypeptide or polynucleotide. In some embodiments, the host cell is a bacterial cell. In some embodiments, the bacterial cell is an E. coli cell.

The terms “culturing” and “culture medium” refers to standard cell culture and recombinant protein expression techniques.

The term “signal sequence” refers to a polypeptide sequence, typically at the N terminus of a polypeptide, expressed in a host cell that directs the polypeptide to a particular cellular compartment. A signal sequence may be a secretion signal to cause the host cell to secrete the polypeptide into the media in which with host cell is cultured. The signal sequence can be the “native” signal sequence, a signal sequence that occurs in nature as part of the polypeptide. The signal sequence can be a sequence that does not naturally occur with the polypeptide as found in nature. Various signal sequences are known and it is within the skill of an ordinary artisan to select an appropriate signal sequence.

The term “polyclonal antibody response” refers to an antibody response comprising antibodies having more than one specificities and/or variation in their antibody sequences.

### DA4B Tetramerization Domain

In one aspect, the disclosure provides a recombinant influenza neuraminidase (NA) tetramer including four copies of a fusion protein. The four copies may be copies of an identical fusion protein (a homomer) or of different fusion proteins (a heteromer). A tetramer comprising different fusion proteins may be termed a “mosaic.” The fusion protein including, in N- to C-terminal order, a DA4B tetramerization domain, an optional linker, and an influenza NA head domain.

In some embodiments, the DA4B tetramerization domain comprises a DA4B polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5. In some embodiments, the DA4B tetramerization domain consists essentially of a DA4B polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5. In some embodiments, the DA4B tetramerization domain consists of a DA4B polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5.

TABLE 1
DA4B tetramerization domain

| SEQ | | |
|---|---|---|
| ID | | |
| NO | Sequence | Length |
| 1 | VIYDVRDDVLTNLTITLDDIKTDLTTLINTR | 50 |
| | VDKLEMMINDNVTTILASG | |
| 2 | DVRDDVLTNLTITLDDIKTDLTTLINTRVDK | 47 |
| | LEMMINDNVTTILASG | |
| 3 | DVLTNLTITLDDIKTDLTTLINTRVDKLEMM | 43 |
| | INDNVTTILASG | |
| 4 | NLTITLDDIKTDLTTLINTRVDKLEMMINDN | 39 |
| | VTTILASG | |
| 5 | TLDDIKTDLTTLINTRVDKLEMMINDNVTTI | 35 |
| | LASG | |

In some embodiments, the tetramer of the DA4B tetramerization domain comprises between 30 and 60 amino residues. In some embodiments, the tetramer of the DA4B tetramerization domain comprises between 30 and 50 amino residues. In some embodiments, the tetramer of the DA4B tetramerization domain comprises between 30 and 40 amino residues. In some embodiments, the tetramer of the DA4B tetramerization domain comprises between 35 and 60 amino residues. In some embodiments, the tetramer of the DA4B tetramerization domain comprises between 35 and 50 amino residues. In some embodiments, the tetramer of the DA4B tetramerization domain comprises between 35 and 45 amino residues.

In some embodiments, the tetramer of the DA4B tetramerization domain comprises at least 30 amino residues. In some embodiments, the tetramer of the DA4B tetramerization domain comprises at least 35 amino residues. In some embodiments, the tetramer of the DA4B tetramerization domain comprises at least 40 amino residues. In some embodiments, the tetramer of the DA4B tetramerization domain comprises at least 45 amino residues. In some embodiments, the tetramer of the DA4B tetramerization domain comprises at least 50 amino residues.

In some embodiments, the tetramer of the DA4B tetramerization domain comprises at most 80 amino residues. In some embodiments, the tetramer of the DA4B tetramerization domain comprises at most 70 amino residues. In some embodiments, the tetramer of the DA4B tetramerization domain comprises at most 65 amino residues. In some embodiments, the tetramer of the DA4B tetramerization domain comprises at most 60 amino residues. In some embodiments, the tetramer of the DA4B tetramerization domain comprises at most 55 amino residues. In some embodiments, the tetramer of the DA4B tetramerization domain comprises at most 50 amino residues. In some embodiments, the tetramer of the DA4B tetramerization domain comprises at most 45 amino residues. In some embodiments, the tetramer of the DA4B tetramerization domain comprises at most 40 amino residues. In some embodiments, the tetramer of the DA4B tetramerization domain comprises at most 35 amino residues.

In some embodiments, the tetramer of the DA4B polypeptide sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5. In some embodiments, the tetramer of the DA4B polypeptide sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1. In some embodiments, the tetramer of the DA4B polypeptide sequence is SEQ ID NO: 5. In some embodiments, the tetramer of the DA4B polypeptide sequence is SEQ ID NO: 1.

### Tetrabrachion Tetramerization Domain

An alpha-helical segment of the tetrabrachion protein (Genbank accession 2211379A, residues 1238-1286, referred to here as “TB”) from the hyperthermophilic archaebacterium Staphyiothermus marinus was previously identified to form a highly stable tetrameric complex with C4 symmetry. Further, TB was found useful for genetic fusion to influenza NA head domains to assist tetramerization and proper folding of soluble NA antigens.

Nonlimiting examples of Tetrabrachion tetramerization domain are provided in Table 2.

TABLE 2
Tetrabrachion (TB) tetramerization domains
(hydrophobic core residues underlined)

| SEQ | | |
|---|---|---|
| ID | | |
| NO | Sequence | Length |
| 6 | IINETADDIVYRLTVIIDDRYESLKNLITL | 50 |
| | RADRLEMIINDNVSTILASG | |
| 7 | ETADDIVYRLTVIIDDRYESLKNLITLRAD | 47 |
| | RLEMIINDNVSTILASG | |
| 8 | DIVYRLTVIIDDRYESLKNLITLRADRLEM | 43 |
| | IINDNVSTILASG | |
| 9 | RLTVIIDDRYESLKNLITLRADRLEMIIND | 39 |
| | NVSTILASG | |
| 10 | IIDDRYESLKNLITLRADRLEMIINDNVST | 35 |
| | ILASG | |

In some embodiments, the Tetrabrachion tetramerization domain comprises a Tetrabrachion polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of sequence in Table 2. In some embodiments, the Tetrabrachion tetramerization domain consists essentially of a Tetrabrachion polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of sequence in Table 2. In some embodiments, the Tetrabrachion tetramerization domain consists of a Tetrabrachion polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of sequence in Table 2.

### Other Types of Tetramerization Domains

In variations, the amino acid substitutions in the head domain described herein are used in polypeptides with other tetramerization domains. In further variations, the polypeptides described herein comprise multiple tandem tetramerization domains. Illustrative alternative tetramerization domain include a measles virus phosphoprotein tetramerization domain or a Sendai virus phosphoprotein tetramerization domain. Further illustrative tetramerization domains include a tetramerization domain from a GCN4 leucine zipper, a bacterial tetrabrachion tetramerization domain or the human vasodilator stimulated phosphoprotein (VASP) tetramerization domain. Illustrative alternative tetramerization domains comprise the tetramerization domain from SEPPALLATA-like MADS domain transcription factor from Arabidopsis thaliana (SMDTF), PiLZ structure from Xhantomonas campestris, or Dictyocaulus viviparus ACE tetramerization domain. Illustrative alternative tetramerization domains comprise a tetramerization domain from a paramyxovirus phosphoprotein (e.g., a Nipah virus phosphoprotein, a Hendra virus phosphoprotein, a respiratory syncytial virus phosphoprotein, human parainfluenza virus (hPIV) phosphoprotein, bovine parainfluenza virus phosphoprotein, a mumps virus phosphoprotein, a Cedar virus phosphoprotein, a Ghana virus phosphoprotein, a Newcastle disease virus phosphoprotein, a canine distemper virus phosphoprotein, or a Peste des petits ruminants virus (PPRV) phosphoprotein). Sequences are provided in Table 3.

TABLE 3
Examples of tetramerization domains

| Tetramerization domain | Sequence |
|---|---|
| human vasodilator | SSSDYSDLQRVKQELLEEVKKELQKVKEEIIEAFVQEL |
| stimulated phosphoprotein | RKRG (SEQ ID NO: 466) |
| (VASP) tetramerization | |
| domain | |
| Tetramerization domain | VELSSQQEYLKLKERYDALQRTQRNLLGEDLGPLSTK |
| from SEPPALLATA-like | ELESLERQLDSSLKQIRALRTQFMLDQSKERMLTETN |
| MADS domain | KTLRLRLADGY (SEQ ID NO: 467) |
| transcription factor from | |
| Arabidopsis thaliana | |
| (SMDTF) | |
| Sendai virus | ENTSSMKEMATLLTSLGVIQSAQEFESSRDASYVFAR |
| phosphoprotein | RALKSANYAEMTFNVCGLILSAEKSSARKVDENKQL |
| tetramerization domain | LKIQESVESFRDIYKRFSEYQKEQNSLLMSNLSTLHIIT |
| (SPP) | D (SEQ ID NO: 468) |
| Nipah Virus | DKYIMPSDDFSNTFFPHDTDRLNYHADHLGDYDLET |
| Phosphoprotein | LCEESVLMGVINSIKLINLDMRLNHIEEQVKEIPKIINK |
| tetramerization domain | LESIDRVLAKTNTALSTIEGHLVSMMIMI |
| | (SEQ ID NO: 469) |
| PiLZ structure from | LLVQRMDAKLDLILALIGRLVRQS |
| Xhantomonas campestris | (SEQ ID NO: 470) |
| Measles virus | GDHYDDELFSDVQDIKTALAKIHEDNQKIISKLESLLL |
| phosphoprotein | LKGEVESIKKQINRQNISISTLEGHLSSIMIAIPGL |
| tetramerization domain | (SEQ ID NO: 471) |
| (MPP) | |
| Dictyocaulus viviparus | AVADVGDPFLLWKQQMDKWQNEYITDWQYHFEQY |
| ACE tetramerization | KKYQTYRHLDSDSCSGS (SEQ ID NO: 472) |
| domain | |

### Neuraminidase Head Domain

Influenza A NAs are divided into subtypes that are clustered into the phylogenetically defined groups 1 (N1, N4, N5, and N8), group 2 (N2, N3, N6, N7, and N9), and bat NA-like (N10 and N11), whereas influenza B NAs are clustered in a separate branch. Neuraminidase is a homotetrameric, type II integral membrane protein with a short cytoplasmic N-terminal domain. The C-terminal catalytic domain folds into a disulfide-stabilized and glycosylated six-bladed beta propeller that is supported by a hypervariable stalk domain. Crystal structures of NA catalytic domains have consistently shown that four identical subunits interact non-covalently to form a globular “head” with four-fold symmetry.

GenBank™ Accession No. AAA43397.1 provides an exemplary amino acid sequence for a human influenza virus neuraminidase. GenBank™ Accession No. ABG23658.1 (GI: 108946273), GenBank™ Accession No. NP 040981.1 (GI: 8486128), GenBank™ Accession No. AAA43412.1 (GI: 324508), GenBank™ Accession No. ABE97720.1 (GI: 93008579), GenBank™ Accession No. ABE97719.1 (GI: 93008577), and GenBank™ Accession No. ABE97718.1 (GI: 93008575) provide exemplary amino acid sequences for human influenza virus neuraminidases. GenBank™ Accession No. CRI06477.1 provides an exemplary amino acid sequence for a swine influenza virus neuraminidase. GenBank™ Accession No. AAQ90293.1 provides an exemplary amino acid sequence for an equine influenza virus neuraminidase. GenBank™ Accession No. AEX30531.1 (GI: 371449652), GenBank™ Accession No. AEX30532.1 (GI: 371449654), GenBank™ Accession No. AIA62041.1 (GI: 641454926), GenBank™ Accession No. AII30325.1 (GI: 670605039), GenBank™ Accession No. AG018161.1 (GI: 513130855), and GenBank™ Accession No. AAS89005.1 (GI: 46360357) provide exemplary amino acid sequences for avian influenza virus neuraminidases. Sequences of influenza virus genes may also be found in the Influenza Research Database. For example, influenza virus neuraminidase sequences may be found in the Influenza Research Database under Accession No. FJ66084 and Accession No. KF90392.

In some embodiments, the neuraminidase head domains described herein are the neuraminidase of influenza A viruses. In some embodiments, the neuraminidase head domain described herein is N1, N2, N3, N4, N5, N6, N7, N8, N9, N10, or N11 influenza virus neuraminidase.

In some embodiments, the neuraminidase head domains described herein are the N1 influenza virus neuraminidase. In some embodiments, the neuraminidase head domain described herein is the neuraminidase of influenza virus A/California/7/2009 (SEQ ID NO: 15). In some embodiments, the neuraminidase head domain described herein is the neuraminidase of influenza virus A/Michigan/45/2015 (SEQ ID NO: 16). In some embodiments, the neuraminidase head domain described herein is the neuraminidase of influenza virus A/Sydney/5/2021 (SEQ ID NO: 17). In some embodiments, the neuraminidase head domain described herein is the neuraminidase of influenza virus A/Wisconsin/588/2019 (SEQ ID NO: 18). In some embodiments, the neuraminidase head domain described herein is the neuraminidase of influenza virus A/Vietnam/1203/2004 (SEQ ID NO: 19). In some embodiments, the neuraminidase head domain described herein is the neuraminidase of influenza virus A/gadwall/North Carolina/AH0182894/2022 (SEQ ID NO: 20). In some embodiments, the neuraminidase head domain described herein is the neuraminidase of influenza virus A/WSN/1933 (SEQ ID NO: 21). In some embodiments, the neuraminidase head domain described herein is the neuraminidase of influenza virus A/Wisconsin/67/2022 (SEQ ID NO: 423).

In some embodiments, the neuraminidase head domains described herein are N2 influenza virus neuraminidase. In some embodiments, the neuraminidase head domain described herein is the neuraminidase of influenza virus A/Darwin/6/2021 (SEQ ID NO: 22). In some embodiments, the neuraminidase head domain described herein is the neuraminidase of influenza virus A/Wisconsin/67/2005 (SEQ ID NO: 23). In some embodiments, the neuraminidase head domain described herein is the neuraminidase of influenza virus A/Moscow/10/1999 (SEQ ID NO: 24). In some embodiments, the neuraminidase head domain described herein is the neuraminidase of influenza virus A/Philippines/2/1982 (SEQ ID NO: 25). In some embodiments, the neuraminidase head domain described herein is the neuraminidase of influenza virus A/Hong Kong/1/1968 (SEQ ID NO: 26).

In some embodiments, the neuraminidase head domains described herein are influenza B viruses. In some embodiments, the neuraminidase head domains described herein are the B/Vic influenza virus neuraminidase. In some embodiments, the neuraminidase head domain described herein is the neuraminidase of influenza virus B/Austria/1359417/2021 (SEQ ID NO: 27). In some embodiments, the neuraminidase head domain described herein is the neuraminidase of influenza virus B/Colorado/06/2017 (SEQ ID NO: 28). In some embodiments, the neuraminidase head domain described herein is the neuraminidase of influenza virus B/Phuket/3073/2013 (SEQ ID NO: 29). In some embodiments, the neuraminidase head domain described herein is the neuraminidase of influenza virus B/Yamagata/16/1988 (SEQ ID NO: 30). In some embodiments, the neuraminidase head domain described herein is the neuraminidase of influenza virus B/Lee/40 (SEQ ID NO: 31). The sequence of the NA head domain from each strain is shown in Table 4. In some embodiments, the influenza NA head domain comprises an N1, N2, and B/Vic influenza virus neuraminidases.

TABLE 4

| SEQ | | |
|---|---|---|
| ID | | |
| NO: | Sequence | Strain |
| 15 | VKLAGNSSLCPVSGWAIYSKDNSVRIGSKGDVFVIREPFISCS | A/California/ |
| | PLECRTFFLTQGALLNDKHSNGTIKDRSPYRTLMSCPIGEVPS | 7/2009 |
| | PYNSRFESVAWSASACHDGINWLTIGISGPDNGAVAVLKYNG | |
| | IITDTIKSWRNNILRTQESECACVNGSCFTVMTDGPSNGQAS | |
| | YKIFRIEKGKIVKSVEMNAPNYHYEECSCYPDSSEITCVCRD | |
| | NWHGSNRPWVSFNQNLEYQIGYICSGIFGDNPRPNDKTGSC | |
| | GPVSSNGANGVKGFSFKYGNGVWIGRTKSISSRNGFEMIWD | |
| | PNGWTGTDNNFSIKQDIVGINEWSGYSGSFVQHPELTGLDCI | |
| | RPCFWVELIRGRPKENTIWTSGSSISFCGVNSDTVGWSWPD | |
| | GAELPFTIDK | |
| 16 | VKLAGNSSLCPVSGWAIYSKDNSVRIGSKGDVFVIREPFISCS | A/Michigan/ |
| | PLECRTFFLTQGALLNDKHSNGTIKDRSPYRTLMSCPIGEVPS | 45/2015 |
| | PYNSRFESVAWSASACHDGINWLTIGISGPDSGAVAVLKYNGI | |
| | ITDTIKSWRNNILRTQESECACVNGSCFTIMTDGPSDGQASY | |
| | KIFRIEKGKIIKSVEMKAPNYHYEECSCYPDSSEITCVCRDN | |
| | WHGSNRPWVSFNQNLEYQMGYICSGVFGDNPRPNDKTGSC | |
| | GPVSSNGANGVKGFSFKYGNGVWIGRTKSISSRKGFEMIWD | |
| | PNGWTGTDNKFSIKQDIVGINEWSGYSGSFVQHPELTGLDCI | |
| | RPCFWVELIRGRPEENTIWTSGSSISFCGVNSDTVGWSWPDG | |
| | AELPFTIDK | |
| 17 | VKLAGNSSLCPVSGWAIYSKDNSVRIGSKGDVFVIREPFISCS | A/Sydney/5/ |
| | PLECRTFFLTQGALLNDKHSNGTIKDRSPYRTLMSCPIGEVPS | 2021 |
| | PYNSRFESVAWSASACHDGTNWLTIGISGPDSGAVAVLKYNG | |
| | IITDTIKSWRNKILRTQESECACVNGSCFTIMTDGPSDGQASY | |
| | KIFRIEKGKIIKSVEMKAPNYHYEECSCYPDSSEITCVCRDN | |
| | WHGSNRPWVSFNQNLEYQMGYICSGVFGDNPRPNDKTGSC | |
| | GPVSSNGANGVKGFSFKYGNGVWIGRTKSISSRKGFEMIWD | |
| | PNGWTGTDNKFSKKQDIVGINEWSGYSGSFVQHPELTGLNC | |
| | IRPCFWVELIRGRPEENTIWTSGSSISFCGVDSDIMGWSWPD | |
| | GAELPFTIDK | |
| 18 | VKLAGNSSLCPVSGWAIYSKDNSVRIGSKGDVFVIREPFISCS | A/Wisconsin/ |
| | PLECRTFFLTQGALLNDKHSNGTIKDRSPYRTLMSCPIGEVPS | 588/2019 |
| | PYNSRFESVAWSASACHDGTNWLTIGISGPDSGAVAVLKYNG | |
| | IITDTIKSWRNKILRTQESECACVNGSCFTIMTDGPSDGQASY | |
| | KIFRIEKGKIIKSVEMKAPNYHYEECSCYPDSSEITCVCRDN | |
| | WHGSNRPWVSFNQNLEYQMGYICSGVFGDNPRPNDKTGSC | |
| | GPVSSNGANGVKGFSFKYGNGVWIGRTKSISSRKGFEMIWD | |
| | PNGWTGTDNKFSKKQDIVGINEWSGYSGSFVQHPELTGLNC | |
| | IRPCFWVELIRGRPEENTIWTSGSSISFCGVDSDIVGWSWPDG | |
| | AELPFTIDK | |
| 19 | VKLAGNSSLCPINGWAVYSKDNSIRIGSKGDVFVIREPFISCS | A/Vietnam/ |
| | HLECRTFFLTQGALLNDKHSNGTVKDRSPHRTLMSCPVGEA | 1203/2004 |
| | PSPYNSRFESVAWSASACHDGTSWLTIGISGPDNGAVAVLKY | |
| | NGIITDTIKSWRNNILRTQESECACVNGSCFTVMTDGPSNGQ | |
| | ASHKIFKMEKGKVVKSVELDAPNYHYEECSCYPNAGEITCV | |
| | CRDNWHGSNRPWVSFNQNLEYQIGYICSGVFGDNPRPNDG | |
| | TGSCGPVSSNGAYGVKGFSFKYGNGVWIGRTKSTNSRSGFE | |
| | MIWDPNGWTETDSSFSVKQDIVAITDWSGYSGSFVQHPELT | |
| | GLDCIRPCFWVELIRGRPKESTIWTSGSSISFCGVNSDTVGWS | |
| | WPDGAELPFTIDK | |
| 20 | VTLAGNSSLCPISGWAIYSKDNGIRIGSKGDVFVIREPFISCSH | A/gadwall/ |
| | LECRTFFLTQGALLNDKHSNGTVKDRSPYRTLMSCPVGEAP | North |
| | SPYNSRFESVAWSASACHDGISWLTIGISGPDNGAVAVLKYN | Carolina/ |
| | GIITDTIKSWRNNILRTQESECACVNGSCFTVMTDGPSNGQA | AH0182894/ |
| | SYKIFKIEKGKVVKSVELNAPNYHYEECSCYPDAGDIMCVC | 2022 |
| | RDNWHGSNRPWVSFNQNLEYQIGYICSGVFGDNPRPNDRT | |
| | GSCSPMSSNGAYGVKGFSFKYGNGVWIGRTKSTSSRSGFEM | |
| | IWDPNGWTETDSSFSVKQDIVEITDWSGYSGSFVQHPELTGL | |
| | DCMRPCFWVELIRGRPKENTIWTSGSSISFCGVNSDTVGWS | |
| | WPDGAELPFTIDK | |
| 21 | VILTGNSSLCPIRGWAIHSKDNGIRIGSKGDVFVIREPFISCSH | A/WSN/1933 |
| | LECRTFFLTQGALLNDKHSRGTFKDRSPYRALMSCPVGEAPS | |
| | PYNSRFESVAWSASACHDGMGWLTIGISGPDDGAVAVLKYN | |
| | GIITETIKSWRKNILRTQESECTCVNGSCFTIMTDGPSDGLAS | |
| | YKIFKIEKGKVTKSIELNAPNSHYEECSCYPDTGKVMCVCR | |
| | DNWHGSNRPWVSFDQNLDYKIGYICSGVFGDNPRPKDGTG | |
| | SCGPVSADGANGVKGFSYKYGNGVWIGRTKSDSSRHGFEM | |
| | IWDPNGWTETDSRFSMRQDVVAMTDRSGYSGSFVQHPELT | |
| | GLDCMRPCFWVELIRGLPEEDAIWTSGSIISFCGVNGDTVD | |
| | WSWPDGAELPFTIDK | |
| 22 | EYRNWSKPQCGITGFAPFSKDNSIRLSAGGDIWVTREPYVSC | A/Darwin/6/ |
| | DLDKCYQFALGQGTTLNNVHSNNTVSDRTPYRTLLMNELG | 2021 (N2) |
| | VPFHLGTKQVCIAWSSSSCHDGKAWLHVCITGDDKNATASFI | |
| | YNGRLVDSVVSWSNDILRTQESECVCINGTCTVVMTDGNAT | |
| | GKADTKILFIEEGKIVHTSKLSGSAQHVEECSCYPRYPGVRC | |
| | VCRDNWKGSNRPIIDINIKDHSIVSRYVCSGLVGDTPRKSDSS | |
| | SSSHCLNPNNEKGDHGVKGWAFDDGNDVWMGRTINETSRL | |
| | GYETFKVVEGWSNPKSKLQINRQVIVDRGDRSGYSGIFSVE | |
| | GKSCINRCFYVELIRGRKEETEVLWTSNSIVVFCGTSGTYGT | |
| | GSWPDGANLSLMHI | |
| 23 | EYRNWSKPQCNITGFAPFSKDNSIRLSAGGDIWVTREPYVSC | A/Wisconsin/ |
| | DPDKCYQFALGQGTTLNNVHSNDTVHDRTPYRTLLMNELG | 67/2005 (N2) |
| | VPFHLGTKQVCIAWSSSSCHDGKAWLHVCVTGDDKNATAS | |
| | FIYNGRLVDSIVSWSKEILRTQESECVCINGTCTVVMTDGSA | |
| | SGKADTKILFIEEGKIVHTSTLSGSAQHVEECSCYPRYLGVR | |
| | CVCRDNWKGSNRPIVDINIKDYSIVSSYVCSGLVGDTPRKND | |
| | SSSSSHCLDPNNEEGGHGVKGWAFDDGNDVWMGRTISEKL | |
| | RSGYETFKVIEGWSNPNSKLQINRQVIVDRGNRSGYSGIFSV | |
| | EGKSCINRCFYVELIRGRKEETEVLWTSNSIVVFCGTSGTYG | |
| | TGSWPDGADINLMPI | |
| 24 | EYRNWSKPQCNITGFAPFSKDNSIRLSAGGDIWVTREPYVSC | A/Moscow/10/ |
| | DPDKCYQFALGQGTTLNNGHSNDTVHDRTPYRTLLMNELG | 1999 (N2) |
| | VPFHLGTKQVCIAWSSSSCHDGKAWLHVCVTGDDENATASF | |
| | IYNGRLVDSIGSWSKKILRTQESECVCINGTCTVVMTDGSAS | |
| | GKADTKILFIEEGKIVHTSPLSGSAQHVEECSCYPRYPGVRC | |
| | VCRDNWKGSNRPIVDINVKDYSIVSSYVCSGLVGDTPRKND | |
| | SSSSSHCLDPNNEEGGHGVKGWAFDDGNDVWMGRTISEKL | |
| | RSGYETFKVIEGWSKPNSKLQINRQVIVDRGNRSGYSGIFSV | |
| | EGKSCINRCFYVELIRGRKQETEVLWTSNSIVVFCGTSGTYG | |
| | TGSWPDGADINLMPI | |
| 25 | EYRNWSKPQCKITGFAPFSKDNSIRLSAGGDIWVTREPYVSC | A/Philippines/ |
| | DPGKCYQFALGQGTTLDNKHSNDTIHDRTPYRTLLMNELGV | 2/1982 (N2) |
| | PFHLGTRQVCIAWSSSSCHDGKAWLHVCITGYDKNATASFIY | |
| | DGRLVDSIGSWSKNILRTQESECVCINGTCTVVMTDGSASER | |
| | ADTKILFIEEGKIVHISPLSGSAQHVEECSCYPRYPGVRCVCR | |
| | DNWKGSNRPVVDINVKDYSIVSSYVCSGLVGDTPRKNDRSS | |
| | SSYCRNPNNEKGNHGVKGWAFDDGNDVWMGRTISEESRSG | |
| | YETFKVIGGWSTPNSKLQINRQVIVDSGNRSGYSGIFSVEGK | |
| | SCINRCFYVELIRGREQETRVWWTSNSIVVFCGTSGTYGTGS | |
| | WPDGADINLMPI | |
| 26 | EYRNWSKPQCQITGFAPFSKDNSIRLSAGGDIWVTREPYVSC | A/Hong Kong/ |
| | DHGKCYQFALGQGTTLDNKHSNDTIHDRIPHRTLLMNELGV | 1/1968 (N2) |
| | PFHLGTRQVCIAWSSSSCHDGKAWLHVCITGDDKNATASFIY | |
| | DGRLVDSIGSWSQNILRTQESECVCINGTCTVVMTDGSASGR | |
| | ADTRILFIEEGKIVHISPLSGSAQHVEECSCYPRYPGVRCICRD | |
| | NWKGSNRPVVDINMEDYSIDSSYVCSGLVGDTPRNDDRSSN | |
| | SNCRNPNNERGNQGVKGWAFDNGDDVWMGRTISKDLRSG | |
| | YETFKVIGGWSTPNSKSQINRQVIVDSDNRSGYSGIFSVEGK | |
| | SCINRCFYVELIRGRKQETRVWWTSNSIVVFCGTSGTYGTGS | |
| | WPDGANINFMPI | |
| 27 | PEPEWTYPRLSCPGSTFQKALLISPHRFGETKGNSAPLIIREPF | B/Austria/ |
| | VACGPNECKHFALTHYAAQPGGYYNGTRGDRNKLRHLISVK | 1359417/2021 |
| | LGKIPTVENSIFHMAAWSGSACHDGKEWTYIGVDGPDNNA | |
| | LLKVKYGEAYTDTYHSYANNILRTQESACNCIGGNCYLMIT | |
| | DGSASGVSECRFLKIREGRIIKEIFPTGRVKHTEECTCGFASN | |
| | KTIECACRDNRYTAKRPFVKLNVETDTAEIRLMCTDTYLDTP | |
| | RPNDGSITGPCESDGDEGSGGIKGGFVHQRMKSKIGRWYSR | |
| | TMSKTERMGMGLYVKYGGDPWADSDALVFSGVMISMKEP | |
| | GWYSFGFEIKDKKCDVPCIGIEMVHDGGKETWHSAATAIYC | |
| | LMGSGQLLWDTITGVDMAL | |
| 28 | PEPEWTYPRLSCPGSTFQKALLISPHRFGETKGNSAPLIIREPF | B/Colorado/ |
| | VACGPNECKHFALTHYAAQPGGYYNGTRGDRNKLRHLISVK | 06/2017 |
| | LGKIPTVENSIFHMAAWSGSACHDGKEWTYIGVDGPDNNA | |
| | LLKVKYGEAYTDTYHSYANNILRTQESACNCIGGNCYLMIT | |
| | DGSASGVSECRFLKIREGRIIKEIFPTGRVKHTEECTCGFASN | |
| | KTIECACRDNRYTAKRPFVKLNVETDTAEIRLMCTDTYLDTP | |
| | RPNDGSITGPCESDGDKGSGGIKGGFVHQRMKSKIGRWYSR | |
| | TMSQTERMGMGLYVKYGGDPWADSDALAFSGVMVSMKEP | |
| | GWYSFGFEIKDKKCDVPCIGIEMVHDGGKETWHSAATAIYC | |
| | LMGSGQLLWDTVTGVDMAL | |
| 29 | PEPEWTYPRLSCPGSTFQKALLISPHRFGETKGNSAPLIIREPF | B/Phuket/ |
| | IACGPKECKHFALTHYAAQPGGYYNGTREDRNKLRHLISVK | 3073/2013 |
| | LGKIPTVENSIFHMAAWSGSACHDGREWTYIGVDGPDSNAL | |
| | LKIKYGEAYTDTYHSYAKNILRTQESACNCIGGDCYLMITDG | |
| | PASGISECRFLKIREGRIIKEIFPTGRVKHTEECTCGFASNKTIE | |
| | CACRDNSYTAKRPFVKLNVETDTAEIRLMCTKTYLDTPRPN | |
| | DGSITGPCESDGDEGSGGIKGGFVHQRMASKIGRWYSRTMS | |
| | KTKRMGMGLYVKYDGDPWTDSEALALSGVMVSMEEPGW | |
| | YSFGFEIKDKKCDVPCIGIEMVHDGGKTTWHSAATAIYCLM | |
| | GSGQLLWDTVTGVNMTL | |
| 30 | SEPEWTYPRLSCQGSTFQKALLISPHRFGETRGNSAPLIIREPF | B/Yamagata/ |
| | IACGPKECKHFALTHYAAQPGGYYNGTREDRNKLRHLISVK | 16/1988 |
| | LGKIPTVENSIFHMAAWSGSACHDGREWTYIGVDGPDSNAL | |
| | IKIKYGEAYTDTYHSYANNILRTQESACNCIGGDCYLMITDG | |
| | SASGISKCRFLKIREGRIIKEIFPTGRVEHTEECTCGFASNKTIE | |
| | CACRDNSYTAKRPFVKLNVETDTAEIRLMCTETYLDTPRPD | |
| | DGSITGPCESNGDKGRGGIKGGFVHQRMASKIGRWYSRTMS | |
| | KTERMGMELYVKYDGDPWTDSDALAPSGVMVSMKEPGW | |
| | YSFGFEIKDKKCDVPCIGIEMVHDGGKKTWHSAATAIYCLM | |
| | GSGQLLWDTVTGVDMAL | |
| 31 | PEPEWTYPRLSCQGSTFQKALLISPHRFGEIKGNSAPLIIREPF | B/Lee/40 |
| | VACGPKECRHFALTHYAAQPGGYYNGTRKDRNKLRHLVSV | |
| | KLGKIPTVENSIFHMAAWSGSACHDGREWTYIGVDGPDND | |
| | ALVKIKYGEAYTDTYHSYAHNILRTQESACNCIGGDCYLMIT | |
| | DGSASGISKCRFLKIREGRIIKEILPTGRVEHTEECTCGFASNK | |
| | TIECACRDNSYTAKRPFVKLNVETDTAEIRLMCTKTYLDTPR | |
| | PDDGSIAGPCESNGDKWLGGIKGGFVHQRMASKIGRWYSRT | |
| | MSKTNRMGMELYVKYDGDPWTDSDALTLSGVMVSIEEPG | |
| | WYSFGFEIKDKKCDVPCIGIEMVHDGGKDTWHSAATAIYCL | |
| | MGSGQLLWDTVTGVDMAL | |
| 423 | VKLAGNSSLCPVSGWAIYSKDNSVRIGSKGDVFVIREPFISCS | A/Wisconsin/ |
| | PLECRTFFLTQGALLNDKHSNGTIKDRSPYRTLMSCPIGEVPS | 67/2022 |
| | PYNSRFESVAWSASACHDGTNWLTIGISGPDSGAVAVLKYNG | |
| | IITDTIKSWRNKILRTQESECACVNGSCFTIMTDGPSDGQASY | |
| | KIFRIEKGKIIKSVEMKAPNYHYEECSCYPDSSEITCVCRDN | |
| | WHGSNRPWVSFNQNLEYQMGYICSGVFGDNPRPNDKTGSC | |
| | GPVLSNGANGVKGFSFKYGNGVWIGRTKSISSRKGFEMIWD | |
| | PNGWTGTDNKFSKKQDIVGINEWSGYSGSFVQHPELTGLNC | |
| | IRPCFWVELIRGRPEENTIWTSGSSISFCGVDSDIMGWSWPD | |
| | GAELPFTIDN | |

In some embodiments, the influenza NA head domain comprises an NA polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15-31 and 423. In some embodiments, the influenza NA head domain consists essentially of an NA polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15-31 and 423. In some embodiments, the influenza NA head domain consists of an NA polypeptide sequence a at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15-31 and 423. In some embodiments, the influenza NA head domain is a head domain of a subtype N1 influenza. In some embodiments, the influenza NA head domain is a head domain of a subtype N2 influenza. In some embodiments, the influenza NA head domain is a head domain of an Influenza virus B.

In some embodiments, the influenza NA head domain comprises one or more, two or more, three or more, four or more amino acid substitutions selected from the group comprising of 99P, 100L, 101A/C, 103N, 105A/S, 105S, 106V, 113E, 114I, 122V, 126P, 131M/Q, 157T, 160P, 161V, 163I/L/S, 164C, 165A/I/S/T/V/Q, 166P/V, 1705/T, 172A, 174V, 175I, 176I, 177I, 196T/S, 203I, 205I, 210G, 257I, 262I, 268M, 408M, 419V, 442I/S, 444A/V, and 453T, numbered relative to SEQ ID NO: 15.

In some embodiments, the influenza NA head domain comprises one or more amino acid substitutions selected from the group consisting of I99P, Y100L, T131Q, C161V, E165S, S172A, V177I, S196T, V205I, Q408M, R419V, and V453T, numbered relative SEQ ID NO: 15. In some embodiments, the influenza NA head domain comprises two or more amino acid substitutions selected from the group consisting of I99P, Y100L, T131Q, C161V, E165S, S172A, V177I, S196T, V205I, Q408M, R419V, and V453T, numbered relative SEQ ID NO: 15. In some embodiments, the influenza NA head domain comprises three or more amino acid substitutions selected from the group consisting of I99P, Y100L, T131Q, C161V, E165S, S172A, V177I, S196T, V205I, Q408M, R419V, and V453T, numbered relative SEQ ID NO: 15. In some embodiments, the influenza NA head domain comprises four or more amino acid substitutions selected from the group consisting of I99P, Y100L, T131Q, C161V, E165S, S172A, V177I, S196T, V205I, Q408M, R419V, and V453T, numbered relative SEQ ID NO: 15. In some embodiments, the influenza NA head domain comprises five or more amino acid substitutions selected from the group consisting of I99P, Y100L, T131Q, C161V, E165S, S172A, V177I, S196T, V205I, Q408M, R419V, and V453T, numbered relative SEQ ID NO: 15.

In some embodiments, the NA head domain comprises a substitution of one or more amino acid at positions I99P, Y100L, T131Q, C161V, E165S, S172A, V177I, S196T, V205I, Q408M, R419V, or V453T relative to a reference sequence according to SEQ ID NO: 15. In some embodiments, the NA head domain comprises a substitution of two or more amino acid at positions I99P, Y100L, T131Q, C161V, E165S, S172A, V177I, S196T, V205I, Q408M, R419V, or V453T relative to a reference sequence according to SEQ ID NO: 15. In some embodiments, the NA head domain comprises a substitution of three or more amino acid at positions I99P, Y100L, T131Q, C161V, E165S, S172A, V177I, S196T, V205I, Q408M, R419V, or V453T relative to a reference sequence according to SEQ ID NO: 15. In some embodiments, the NA head domain comprises a substitution of four or more amino acid at positions I99P, Y100L, T131Q, C161V, E165S, S172A, V177I, S196T, V205I, Q408M, R419V, or V453T relative to a reference sequence according to SEQ ID NO: 15. In some embodiments, the NA head domain comprises a substitution of five or more amino acid at positions I99P, Y100L, T131Q, C161V, E165S, S172A, V177I, S196T, V205I, Q408M, R419V, or V453T relative to a reference sequence according to SEQ ID NO: 15.

In some embodiments, the tetramer of the influenza NA head domain comprises the amino acid substitutions, numbered relative SEQ ID NO: 15, selected from:
a. I99P, C161V, E165S, S172A, S196T,
b. I99P, T131Q, C161V, E165S, S172A, S196T,
c. I99P, T131Q, C161V, E165S, S172A, V177I, S196T,
d. I99P, T131Q, C161V, E165S, S172A, S196T, V453T,
e. I99P, T131Q, C161V, E165S, S172A, V177I, S196T, V205I,
f. I99P, Y100L, T131Q, C161V, E165S, S172A, V177I, S196T, V205I, Q408M, R419V,
g. I99P, C161V, E165S, S172A, V177I, S196T,
h. I99P, C161V, S172A, S196T, or
i. I99P, C161V, S172A, V177I, S196T.

In some embodiments, the influenza NA head domain has substitutions of amino acids at positions I99P, C161V, E165S, S172A, and S196T relative to a reference sequence according to SEQ ID NO: 15. In some embodiments, the influenza NA head domain has substitutions of amino acids at positions I99P, T131Q, C161V, E165S, S172A, and S196T relative to a reference sequence according to SEQ ID NO: 15. In some embodiments, the influenza NA head domain has substitutions of amino acids at positions I99P, T131Q, C161V, E165S, S172A, V177I, and S196T relative to a reference sequence according to SEQ ID NO: 15. In some embodiments, the influenza NA head domain has substitutions of amino acids at positions I99P, T131Q, C161V, E165S, S172A, S196T, and V453T relative to a reference sequence according to SEQ ID NO: 15. In some embodiments, the influenza NA head domain has substitutions of amino acids at positions I99P, T131Q, C161V, E165S, S172A, V177I, S196T, and V205I relative to a reference sequence according to SEQ ID NO: 15. In some embodiments, the influenza NA head domain has substitutions of amino acids at positions I99P, Y100L, T131Q, C161V, E165S, S172A, V177I, S196T, V205I, Q408M, and R419V relative to a reference sequence according to SEQ ID NO: 15. In some embodiments, the influenza NA head domain has substitutions of amino acids at positions I99P, C161V, E165S, S172A, V177I, and S196T relative to a reference sequence according to SEQ ID NO: 15. In some embodiments, the influenza NA head domain has substitutions of amino acids at positions I99P, C161V, S172A, and S196T relative to a reference sequence according to SEQ ID NO: 15. In some embodiments, the influenza NA head domain has substitutions of amino acids at positions I99P, C161V, S172A, V177I, and S196T relative to a reference sequence according to SEQ ID NO: 15. Illustrative combinations of mutations are identified in the below table:

TABLE 5
Illustrative combinations of mutations

| Mutation combination | Name |
|---|---|
| I99P, C161V, E165S, S172A, S196T | 5mut |
| I99P, T131Q, C161V, E165S, S172A, S196T | 6mutA |
| I99P, T131Q, C161V, E165S, S172A, V177I, S196T | 7mutA |
| I99P, T131Q, C161V, E165S, S172A, S196T, V453T | 7mutB |
| I99P, T131Q, C161V, E165S, S172A, V177I, S196T, V205I | 8mut |
| I99P, Y100L, T131Q, C161V, E165S, S172A, V177I, S196T, | 11mut |
| V205I, Q408M, R419V | |
| I99P, C161V, E165S, S172A, V177I, S196T | 6mutB |
| I99P, C161V, S172A, S196T | |
| I99P, C161V, S172A, V177I, S196T | |

In each case the listed amino acid substitutions may be the only amino acid substitutions relative to a native neuraminidase sequence, or they may be combined with other amino acid substitutions. The other amino acid substitutions may be substitutions described herein or other substitutions.

In some embodiments, a cysteine substitution should be combined with its cognate substitution to make disulfide bond. The introduced cysteine residue pairs allows disulfide bond formation that stabilize the protein's conformation or oligomeric state.

### Linkers

In some embodiments, a linker connects the tetramerization domain to a NA head domain. The linker may be any chemical linkage including but not limited to a polypeptide used to form a N-terminal or C-terminal fusion of the tetramerization domain to the NA head domain.

A wide variety of polypeptide sequences can be used to link the tetramerization domain and the NA head domains. In some cases, the linker comprises that polypeptide sequence. Any suitable linker polypeptide can be used. In some embodiments, the linker imposes a rigid relative orientation of the tetramerization domain to the NA head domain. In some embodiments, the linker flexibly links the tetramerization domain to the NA head domain.

In some embodiments, the encoded polypeptide includes a linker between regions. A wide variety of polypeptide sequences can be used and are well known in the art. In some embodiments, the tetramer of the linker is polypeptide bond. In some embodiments, the tetramer of the linker is SG. In some embodiments, the tetramer of the linker is GGSGGSGG (SEQ ID NO: 11). In some embodiments, the tetramer of the linker is ATPRGG (SEQ ID NO: 12). In some embodiments, the tetramer of the linker is ATPRGSGG (SEQ ID NO: 13). In some embodiments, the tetramer of the linker is GGATPRGSGG (SEQ ID NO: 14).

In some embodiments, the linker may comprise a Gly-Ser linker (i.e., a linker consisting of glycine and serine residues) of any suitable length. In some embodiments, the Gly-Ser linker may be 2, 3, 4, 5, 6, 7, 8, 9, 10, 11, 12, 13, 14, 15, 16, 17, 18, 19, 20, or more amino acids in length. In some embodiments, the linker sequence comprises the sequence GSGGSGSGSGGS (SEQ ID NO: 481). In some embodiments, the Gly-Ser linker may comprise or consist of the amino acid sequence of GSGGSGSGSGGSGSG (SEQ ID NO: 482), GGSGGSGS (SEQ ID NO: 483), or GSGGSGSG (SEQ ID NO: 484). In some embodiments, the linker comprises the sequence GSGSGSG (SEQ ID NO: 485). In some embodiments, the linker comprises the sequence GSGSGSGSGSGSGSG (SEQ ID NO: 486).

### Fusion Proteins

In another aspect, the disclosure provides a fusion protein comprising a DA4B tetramerization domain and a heterologous polypeptide, wherein the DA4B tetramerization domain comprises, consists essentially of, or consists of a DA4B polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5.

In some embodiments, the fusion protein comprises the tetramerization domain of the disclosure, the linker of the disclosure, and the NA head domain of the disclosure.

In some embodiment, the fusion protein comprises of a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 32-422. In some embodiment, the fusion protein consists of a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 32-422. In some embodiment, the fusion protein consists essentially of a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 32-422.

In some embodiments, the fusion protein comprises a DA4B tetramer, a linker, and an influenza A/California/7/2009 NA head domain (collectively, SEQ ID NO: 15), or antigenic variant thereof. In some embodiments, the fusion protein comprises a DA4B tetramer, a linker, and an influenza A/Michigan/45/2015 NA head domain (collectively, SEQ ID NO: 16), or antigenic variant thereof. In some embodiments, the fusion protein comprises a DA4B tetramer, a linker, and an influenza A/Sydney/5/2021 NA head domain (collectively, SEQ ID NO: 17), or antigenic variant thereof. In some embodiments, the fusion protein comprises a DA4B tetramer, a linker, and an influenza A/Wisconsin/588/2019 NA head domain (collectively, SEQ ID NO: 18), or antigenic variant thereof. In some embodiments, the fusion protein comprises a DA4B tetramer, a linker, and an influenza A/Vietnam/1203/2004 NA head domain (collectively, SEQ ID NO: 19), or antigenic variant thereof. In some embodiments, the fusion protein comprises a DA4B tetramer, a linker, and an influenza A/gadwall/North Carolina/AH0182894/2022 NA head domain (collectively, SEQ ID NO: 20), or antigenic variant thereof. In some embodiments, the fusion protein comprises a DA4B tetramer, a linker, and an influenza A/WSN/1933 NA head domain (collectively, SEQ ID NO: 21), or antigenic variant thereof. In some embodiments, the fusion protein comprises a DA4B tetramer, a linker, and an influenza A/Darwin/6/2021 NA head domain (collectively, SEQ ID NO: 22), or antigenic variant thereof. In some embodiments, the fusion protein comprises a DA4B tetramer, a linker, and an influenza A/Wisconsin/67/2005 NA head domain (collectively, SEQ ID NO: 23), or antigenic variant thereof. In some embodiments, the fusion protein comprises a DA4B tetramer, a linker, and an influenza A/Moscow/10/1999 NA head domain (collectively, SEQ ID NO: 24), or antigenic variant thereof. In some embodiments, the fusion protein comprises a DA4B tetramer, a linker, and an influenza A/Philippines/2/1982 NA head domain (collectively, SEQ ID NO: 25), or antigenic variant thereof. In some embodiments, the fusion protein comprises a DA4B tetramer, a linker, and an influenza A/Hong Kong/1/1968 NA head domain (collectively, SEQ ID NO: 26), or antigenic variant thereof. In some embodiments, the fusion protein comprises a DA4B tetramer, a linker, and an influenza B/Austria/1359417/2021 NA head domain (collectively, SEQ ID NO: 27), or antigenic variant thereof. In some embodiments, the fusion protein comprises a DA4B tetramer, a linker, and an influenza B/Colorado/06/2017 NA head domain (collectively, SEQ ID NO: 28), or antigenic variant thereof. In some embodiments, the fusion protein comprises a DA4B tetramer, a linker, and an influenza B/Phuket/3073/2013 NA head domain (collectively, SEQ ID NO: 29), or antigenic variant thereof. In some embodiments, the fusion protein comprises a DA4B tetramer, a linker, and an influenza B/Yamagata/16/1988 NA head domain (collectively, SEQ ID NO: 30), or antigenic variant thereof. In some embodiments, the fusion protein comprises a DA4B tetramer, a linker, and an influenza B/Lee/40 NA head domain (collectively, SEQ ID NO: 31), or antigenic variant thereof.

### Neuraminidase Tetramers

In some embodiments, the disclosure describes the neuraminidase head domains. In embodiments, the neuraminidase head domains are in a closed conformation. The tetramer may be formed by expression of multiple copies of a polypeptide chain, or copies of a mixed population of several polypeptide chains for a so-called “mosaic” vaccine, which may spontaneously self-assemble in sets of four, under suitable conditions, to form tetramers (that is, complexes having four copies of the polypeptide chain or chains). Self-assembly may be driven by interactions between the NA head domains, between the tetramerization domains, or both.

As used herein, “open conformation” refers to head domains that do not form a single, compact structure. These NAs instead exhibited a clover-like appearance characterized by a thin stalk tethered to independent densities corresponding to the four globular head domains. As used herein, “closed conformation” refers to head domains packed tightly and resemble the four-fold symmetric structure classically observed by X-ray crystallography.

Several monoclonal antibodies (mAbs) are known to be used to assess the conformational state of NA. A mAb that binds across the interface of two neighboring protomers in the closed, crystallographically observed C4-symmetric configuration. In some embodiments, head domains from NA subtypes form closed tetramers naturally. In some embodiments, head domains from NA subtypes form open tetramers naturally. In some embodiments, head domains from NA subtypes form mixtures of open and closed tetramers.

Negative stain electron microscopy (NS-EM) with two-dimensional (2D) class averaging, can be used to analyze structural heterogeneity among the various recombinant NAs. Cryoelectron microscopy (cryo-EM) with 2D classification of the data can also be used to analyze structural conformation of NAs.

In some embodiments, the influenza NA head domain comprises an N1 influenza virus neuraminidase. In some embodiments, the influenza NA head domain comprises an N2 influenza virus neuraminidase. In some embodiments, the influenza NA head domain comprises a B/Vic influenza virus neuraminidase. In some embodiments, the influenza NA head domain comprises the combination of N1, N2, and B/Vic influenza virus neuraminidases.

In some embodiments, amino acid substitutions in NA head domain result in a recombinant influenza neuraminidase (NA) tetramer that is partially, substantially, or completely in a closed conformation. In some embodiments, amino acid substitutions in NA head domain result in a recombinant influenza neuraminidase (NA) tetramer that is partially, substantially, or completely in an open conformation. In some embodiments, amino acid substitutions result in about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% closed-conformation tetramers. In some embodiments, amino acid substitutions result in at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% closed-conformation tetramers.

In some embodiments, amino acid substitutions result in about 45% to about 95%, about 50% to about 95%, about 55% to about 95%, about 60% to about 95%, about 65% to about 95%, about 70% to about 95%, about 75% to about 95%, about 80% to about 95%, or about 85% to about 95% closed-conformation tetramers. In some embodiments, amino acid substitutions result in about 45% to about 90%, about 50% to about 90%, about 55% to about 90%, about 60% to about 90%, about 65% to about 90%, about 70% to about 90%, about 75% to about 90%, about 80% to about 90%, or about 85% to about 90% closed-conformation tetramers.

In some embodiments, amino acid substitutions result in about 45% to about 80%, about 50% to about 80%, about 55% to about 80%, about 60% to about 80%, about 65% to about 80%, about 70% to about 80%, or about 75% to about 80% closed-conformation tetramers. In some embodiments, amino acid substitutions result in about 45% to about 70%, about 50% to about 70%, about 55% to about 70%, about 60% to about 70%, or about 65% to about 70% closed-conformation tetramers.

In some embodiments, amino acid substitutions result in about 45% to about 100% closed-conformation tetramer particles. In some embodiments, amino acid substitutions result in about 55% to about 100% closed-conformation tetramer particles. In some embodiments, amino acid substitutions result in about 65% to about 100% closed-conformation tetramer particles. In some embodiments, amino acid substitutions result in about 75% to about 100% closed-conformation tetramer particles. In some embodiments, amino acid substitutions result in about 80% to about 100% closed-conformation tetramer particles. In some embodiments, amino acid substitutions result in about 85% to about 100% closed-conformation tetramer particles. In some embodiments, amino acid substitutions result in about 90% to about 100% closed-conformation tetramer particles. In some embodiments, amino acid substitutions result in about 95% to about 100% closed-conformation tetramer particles. In some embodiments, amino acid substitutions result in about 50% to about 95% closed-conformation tetramer particles. In some embodiments, amino acid substitutions result in about 60% to about 95% closed-conformation tetramer particles. In some embodiments, amino acid substitutions result in about 70% to about 95% closed-conformation tetramer particles. In some embodiments, amino acid substitutions result in about 80% to about 95% closed-conformation tetramer particles. In some embodiments, amino acid substitutions result in about 90% to about 95% closed-conformation tetramer particles.

In some embodiments, the disclosure describes the neuraminidase tetramer that has increased thermal stability in the presence of chelating agent compared to a tetramer with tetrabrachion tetramerization domain.

As used herein, the term “thermal stability” or “thermostability” of a protein refers to protein stability in terms of denaturation due to temperature alteration and can be determined by the kinetics of protein denaturation during heating. Thermal stability can be evaluated by measuring the melting temperature (Tₘ) or onset temperature (Tₒₙₛₑₜ) of a composition of the invention using any suitable technique. The melting temperature is the temperature at which the first derivative of the melting curve reaches a local maximum. The onset temperature is the temperature at which a linear regression of the baseline intersects with a linear regression of the melting transition.

In some embodiments, a nano differential scanning fluorimetry (nanoDSF) can be used to determine the thermal stability by monitoring intrinsic tryptophan fluorescence. In some embodiments, static light scattering (SLS) can be used to reveal an aggregation temperature.

In some embodiments, thermal stability can be evaluated by analytical spectroscopy. An exemplary analytical spectroscopy method is Circular Dichroism (CD) spectroscopy. CD spectroscopy measures the optical activity of a composition as a function of increasing temperature. CD spectroscopy measures differences in the absorption of left-handed polarized light versus right-handed polarized light which arise due to structural asymmetry. A disordered or unfolded structure results in a CD spectrum very different from that of an ordered or folded structure. The CD spectrum reflects the sensitivity of the proteins to the denaturing effects of increasing temperature and is therefore indicative of a protein's thermal stability (see, e.g., van Mierlo and Steemsma, J. BiotechnoL, 79(3):281-98, 2000).

Another exemplary analytical spectroscopy method for measuring thermal stability is Fluorescence Emission Spectroscopy. Fluorescence-based methods to evaluate thermal stability monitor changes in the fluorescence of intrinsic fluorophores (e.g., tryptophan and tyrosine amino acids) or extrinsic fluorophores (e.g., ANS or SYPRO Orange) upon thermal unfolding (see, e.g., Niesen F. PL, Berglund H. and Vedadi M.: The use of differential scanning fluorimetry to detect ligand interactions that promote protein stability. Nature Protocols 2007, 2:2212-21.) Yet another exemplary analytical spectroscopy method for measuring antibody, light or heavy chain thermal stability is Nuclear Magnetic Resonance (NMR) spectroscopy (see, e.g., van Mierlo and Steemsma, J. Biotechnol., 79(3):281-98, 2000).

The NA head features deep pockets on the exterior face of each protomer comprising the catalytic residues. NA contains several Ca2+ binding sites, one of which is highly conserved and supports the periphery of the catalytic pocket on each protomer, while another site at the four-fold symmetry axis is frequently, but not universally, observed across various subtypes. Ca2+ ions have been shown to assist catalytic activity and have been resolved to varying degrees in crystal structures, with reports and predictions of increased structural flexibility in their absence. Because NA contains an interprotomeric Ca2+ binding site that thermally stabilizes the protein, addition of calcium salts, e.g., CaCl₂, in presence or absence of chelating agent (e.g. EDTA), on thermal stability of recombinant NAs can be evaluated.

In some embodiments, the chelating agent is BAPTA-AM (N,N′-[1,2-ethanediylbis(oxy-2,1-phenylene)]bis[N-[2-[(acetyloxy)methoxy]-2-oxoethyl]-1,1′-bis[(acetyloxy)methyl] ester-glycine)), EDTA (Ethylenediamine tetraacetic acid), or EGTA (aminopolycarboxylic acid).

In some embodiments, the melting temperature (Tm) of a NA tetramer as described herein is about 35° C. In some embodiments, the melting temperature (Tm) is about 40° C. In some embodiments, the melting temperature (Tm) is about 45° C. In some embodiments, the melting temperature (Tm) is about 50° C. In some embodiments, the melting temperature (Tm) is about 55° C. In some embodiments, the melting temperature (Tm) is about 60° C. In some embodiments, the melting temperature (Tm) is about 65° C. In some embodiments, the melting temperature (Tm) is about 70° C. In some embodiments, the melting temperature (Tm) is about 75° C.

In some embodiments, the melting temperature (Tm) of a NA tetramer as described herein is at least about 35° C. In some embodiments, the melting temperature (Tm) is at least about 40° C. In some embodiments, the melting temperature (Tm) is at least about 45° C. In some embodiments, the melting temperature (Tm) is at least about 50° C. In some embodiments, the melting temperature (Tm) is at least about 55° C. In some embodiments, the melting temperature (Tm) is at least about 60° C. In some embodiments, the melting temperature (Tm) is at least about 65° C. In some embodiments, the melting temperature (Tm) is at least about 70° C. In some embodiments, the melting temperature (Tm) is at least about 75° C.

In some embodiments, the melting temperature (Tm) is from 35° C. to 75° C. In some embodiments, the melting temperature (Tm) is from 40° C. to 75° C. In some embodiments, the melting temperature (Tm) is from 45° C. to 75° C. In some embodiments, the melting temperature (Tm) is from 50° C. to 75° C. In some embodiments, the melting temperature (Tm) is from 55° C. to 75° C. In some embodiments, the melting temperature (Tm) is from 60° C. to 75° C. In some embodiments, the melting temperature (Tm) is from 65° C. to 75° C. In some embodiments, the melting temperature (Tm) is from 70° C. to 75° C. In some embodiments, the melting temperature (Tm) is from 35° C. to 70° C. In some embodiments, the melting temperature (Tm) is from 40° C. to 70° C. In some embodiments, the melting temperature (Tm) is from 45° C. to 70° C. In some embodiments, the melting temperature (Tm) is from 50° C. to 70° C. In some embodiments, the melting temperature (Tm) is from 55° C. to 70° C. In some embodiments, the melting temperature (Tm) is from 60° C. to 70° C. In some embodiments, the melting temperature (Tm) is from 65° C. to 70° C. In some embodiments, the melting temperature (Tm) is from 35° C. to 65° C. In some embodiments, the melting temperature (Tm) is from 40° C. to 65° C. In some embodiments, the melting temperature (Tm) is from 45° C. to 65° C.° C. In some embodiments, the melting temperature (Tm) is from 50° C. to 65° C. In some embodiments, the melting temperature (Tm) is from 55° C. to 65° C. In some embodiments, the melting temperature (Tm) is from 60° C. to 65° C. In some embodiments, the melting temperature (Tm) is from 35° C. to 60° C. In some embodiments, the melting temperature (Tm) is from 40° C. to 60° C. In some embodiments, the melting temperature (Tm) is from 45° C. to 60° C. In some embodiments, the melting temperature (Tm) is from 50° C. to 60° C. In some embodiments, the melting temperature (Tm) is from 55° C. to 60° C.

In some embodiments, the melting temperature (Tm) of an expressed polypeptide as described herein is about 0.1° C. increased. In some embodiments, the melting temperature (Tm) of an expressed polypeptide as described herein is about 0.2° C. increased. In some embodiments, the melting temperature (Tm) of an expressed polypeptide as described herein is about 0.3° C. increased. In some embodiments, the melting temperature (Tm) of an expressed polypeptide as described herein is about 0.4° C. increased. In some embodiments, the melting temperature (Tm) of an expressed polypeptide as described herein is about 0.5° C. increased. In some embodiments, the melting temperature (Tm) of an expressed polypeptide as described herein is about 0.6° C. increased. In some embodiments, the melting temperature (Tm) of an expressed polypeptide as described herein is about 0.7° C. increased. In some embodiments, the melting temperature (Tm) of an expressed polypeptide as described herein is about 0.8° C. increased. In some embodiments, the melting temperature (Tm) of an expressed polypeptide as described herein is about 0.9° C. increased. In some embodiments, the melting temperature (Tm) of an expressed polypeptide as described herein is about 1.0° C. increased. In some embodiments, the melting temperature (Tm) of an expressed polypeptide as described herein is about 1.5° C. increased. In some embodiments, the melting temperature (Tm) of an expressed polypeptide as described herein is about 2.0° C. increased. In some embodiments, the melting temperature (Tm) of an expressed polypeptide as described herein is about 3.0° C. increased. In some embodiments, the melting temperature (Tm) of an expressed polypeptide as described herein is about 4.0° C. increased. In some embodiments, the melting temperature (Tm) of an expressed polypeptide as described herein is about 5.0° C. increased or more.

In some embodiments, the disclosure describes the neuraminidase tetramer that has increased interaction for a neuraminidase inhibitor compared to a tetramer with a tetrabrachion tetramerization domain.

Binding affinity for a target antigen can be measured or determined by standard antibody-antigen assays, such as competitive assays, saturation assays, or immunoassays such as ELISA (enzyme-linked immunoassay) or RIA (radioimmunoassay).

In some embodiments, the neuraminidase inhibitor is oseltamivir (Tamiflu®), zanamivir (Relenza), and Peramivir ((1S,2S,3S,4R)-3-[(1S)-1-acetamido-2-ethyl-butyl]-4-(diaminomethylideneamino)-2-hydroxycyclopentane- 1-carboxylic acid).

In some embodiments, the neuraminidase tetramer expressed in recombinant cell culture has an increased yield compared to a tetramer with a tetrabrachion tetramerization domain.

Yield can be measured by techniques well-known in the art, such as immobilized metal affinity (IMAC) followed by size-exclusion chromatography (SEC) purification or ultraviolet-visible (UV-Vis) spectroscopy, or by western blot analysis.

In some embodiments, the final yield is 1.5 times higher for DA4B-based tetramer compared to a tetramer with a tetrabrachion tetramerization domain. In some embodiments, the final yield is 2 times higher for DA4B-based tetramer compared to a tetramer with a tetrabrachion tetramerization domain. In some embodiments, the final yield is 3 times higher for DA4B-based tetramer. In some embodiments, the final yield is 4 times higher for DA4B-based tetramer. In some embodiments, the final yield is 5 times higher for DA4B-based tetramer. In some embodiments, the final yield is 10 times higher for DA4B-based tetramer. In some embodiments, the final yield is between 1.5 and 5, between 2 and 5, between 3 and 5, or between 4 or 5 times higher for DA4B-based tetramer compared to a tetramer with a tetrabrachion tetramerization domain. In some embodiments, the final yield is between 1.5 and 4, between 2 and 4, or between 3 and 4 times higher for DA4B-based tetramer compared to a tetramer with a tetrabrachion tetramerization domain.

### Polynucleotides

In another aspect, the disclosure provides a polynucleotide encoding any of the polypeptides described herein. The polynucleotide sequence may comprise RNA or DNA. “Isolated polynucleotides” as used herein refers to those that have been removed from their normal surrounding nucleic acid sequences in the genome or in cDNA sequences. Such polynucleotide sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what polynucleotide sequences will encode the proteins of the disclosure. In some embodiments, the polynucleotide is a messenger RNA (mRNA). In some embodiments, the polynucleotide is a messenger RNA (mRNA). In some embodiments, the polynucleotide is a cDNA. In some embodiments, the polynucleotide is a plasmid.

### Signal Sequences

In some embodiments, the encoded polypeptide includes a peptide region that is a signal sequence. Signal sequences are well known in the art. The signal sequence may be the native signal sequence or can be replaced with another signal sequence. Signal sequence functions to prompt a cell to translocate the protein, usually to the cellular membrane. The core of the signal sequence often contains a long stretch of hydrophobic amino acids (about 5-16 residues long) that has a tendency to form a single alpha-helix and is also referred to as the “h-region”. In addition, many signal sequence begin with a short positively charged stretch of amino acids, which may help to enforce proper topology of the polypeptide during translocation by what is known as the positive-inside rule. Because of its close location to the N-terminus it is called the “n-region”. At the end of the signal sequence there is typically a stretch of amino acids that is recognized and cleaved by signal peptidase.

In some embodiments, the polypeptide does not include a signal sequence.

Nonlimiting examples of signal sequence are provided in Table 6.

TABLE 6

| Sequence | SEQ ID NO: |
|---|---|
| MGILPSPGMPALLSLVSLLSVLLMGCVAETG | 473 |
| MELLILKANAITTILTAVTFCFASG | 474 |
| MSWKVMIIISLLITPQHGL | 475 |
| MKAILVVLLYTFTTANA | 476 |
| MPISILLIITTMIMASHC | 477 |
| MFVFLVLLPLVSSQC | 478 |
| MVPQVLLFVPLLGFSLCFG | 479 |

The native signal sequence is post-translationally cleaved when the protein is expressed. The native signal sequence may be replaced with another signal sequence for expression of the NA head domain comprising tetramerization domain. In some embodiments, no signal sequence is used.

In some embodiments, the present disclosure may optionally contain a poly-His tag, HHHHHH (SEQ ID NO: 480).

### Delivery Vehicles

In another aspect, the disclosure provides a delivery vehicle comprising the polynucleotide described herein.

In some embodiments, the delivery vehicle is a non-viral vector. In some embodiments, the delivery vehicle is a lipid nanoparticle (LNP). In some embodiments, the delivery vehicle is a liposome. In some embodiments, the delivery vehicle is a polymeric-non-viral vector, such as spermine, polyethyleneimine, chitosan, or polyurethane. In some embodiments, the delivery vehicle is a polymer delivery system, such as poly-amido-amine (PAA), poly-beta aminoesters (PBAEs) or polyethylenimine (PEI). In some embodiments, the delivery vehicle is a ferritin nanoparticle.

Lipid nanoparticles may be, e.g., microspheres (including unilamellar and multilamellar vesicles, e.g., liposomes), a dispersed phase in an emulsion, micelle or an internal phase in a suspension.

In various embodiments, the lipid nanoparticles have a mean diameter from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm. In some embodiments, the LNPs are substantially non-toxic. In certain embodiments, polynucleotides, when present in the LNPs, are resistant in aqueous solution to degradation with a nuclease. Lipids and LNPs comprising polynucleotide and their method of preparation are described in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the contents of each of which are incorporated by reference herein.

Further exemplary lipids and LNPs and their manufacture are known in the art—for example in U.S. Pat. Appl. Pub. No. U.S. 2012/0276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference herein. Lipids and their manufacture can be found, for example, in U.S. Pub. No. 2015/0376115 and 2016/0376224, the contents of which are incorporated by reference herein.

### Pharmaceutical Compositions

In another aspect, the disclosure provides a pharmaceutical composition, comprising the polypeptides and polynucleotides described herein and a pharmaceutically acceptable carrier.

### Adjuvants

In some embodiments, the pharmaceutical composition is used as a vaccine. In some embodiments, vaccine compositions may include an immunological adjuvant. Illustrative adjuvants include the following: mineral-containing compositions; oil emulsions; squalene emulsions; saponin formulations; virosomes and virus-like particles; bacterial or microbial derivatives; bioadhesives and mucoadhesives; liposomes; polyoxyethylene ether and polyoxyethylene ester formulations; polyphosphazene (pcpp); muramyl peptides; imidazoquinolone compounds; thiosemicarbazone compounds; tryptanthrin compounds; human immunomodulators; lipopeptides; benzonaphthyridines; microparticles; immunostimulatory polynucleotide (such as RNA or DNA; e.g., CpG-containing oligonucleotides).

For example, the pharmaceutical composition may include an aluminum salt adjuvant, an oil in water emulsion (e.g., an oil-in-water emulsion comprising squalene, such as MF59 or AS03), a TLR7 agonist (such as imidazoquinoline or imiquimod), or a combination thereof. Suitable aluminum salts include hydroxides (e.g., oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. The salts can take any suitable form (e.g., gel, crystalline, amorphous, etc.), with adsorption of antigen to the salt being an example. The concentration of Al⁺⁺⁺ in a composition for administration to a patient may be less than 5 mg/ml e.g., <4 mg/ml, <3 mg/ml, <2 mg/ml, <1 mg/ml, etc. A preferred range is between 0.3 and 1 mg/ml. A maximum of 0.85 mg/dose is preferred. Aluminum hydroxide and aluminum phosphate adjuvants are suitable for use with the disclosure.

Exemplary adjuvants that may be used in a pharmaceutical composition provided herein include, but are not limited to, 3M-052, Adju-Phos™, Alhydrogel™, Adjumer™, albumin-heparin microparticles, Algal Glucan, Algammulin, Alum, Antigen Formulation, AS-2 adjuvant, ASO1, ASO3, autologous dendritic cells, autologous PBMC, Avridine™, B7-2, BAK, BAY R1005, BECC TLR-4 agonists, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin Al-subunit-Protein A D-fragment fusion protein, CpG, CPG-1018, CPG-1018 plus aluminum salt, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™, immunoliposomes containing antibodies to costimulatory molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, Matrix-M™ adjuvant, MF59, MONTANIDE ISA 51, MONTANIDE ISA 720, MPL.™., MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribi like adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, SWE, Tetanus toxoid (TT), Theramide™, Threonyl muramyl dipeptide (TMDP), Ty Particles, and Walter Reed Liposomes.

In some embodiments, the adjuvant is an aluminum hydroxide gel (e.g., Alhydrogel™) In embodiments, the adjuvant is SWE. In embodiments, the adjuvant is MF59. MF59 is an oil-in-water emulsion containing squalene (4.3%) in citric acid buffer with stabilizing nonionic surfactants Tween 80 (0.5%) and Span 85 (0.5%). MF59 has been shown to be well-tolerated in humans and is used in vaccines against seasonal influenza (see Ko and Kang, Hum Vaccin Immunother. 2018; 14(12): 3041-3045; U.S. Pat. No. 6,299,884).

For example, the composition may include an aluminum salt adjuvant, an oil in water emulsion (e.g., an oil-in-water emulsion comprising squalene, such as MF59, SWE, or AS03), a TL4 agonist, a TLR9 agonist (such as CpG oligodeoxynucleotides), a TLR7 agonist (such as imidazoquinoline or imiquimod), or a combination thereof. In some embodiments, the adjuvant is a combination of an aluminum salt and CPG1018. Suitable aluminum salts include hydroxides (e.g., oxyhydroxides), phosphates (e.g., hydroxyphosphates, orthophosphates), (e.g. see chapters 8 & 9 of Vaccine Design. (1995) eds. Powell & Newman. ISBN: 030644867X. Plenum), or mixtures thereof. The salts can take any suitable form (e.g., gel, crystalline, amorphous, etc.), with adsorption of antigen to the salt being an example. The concentration of Al+++ in a composition for administration to a patient may be less than 5 mg/ml e.g., <4 mg/ml, <3 mg/ml, <2 mg/ml, <1 mg/ml, etc. A preferred range is between 0.3 and 1 mg/ml. A maximum of 0.85 mg/dose is preferred. Aluminum hydroxide and aluminum phosphate adjuvants are suitable for use with the disclosure. In some embodiments, a pharmaceutical composition provided herein comprises aluminum hydroxide as an adjuvant.

In some embodiments, the adjuvant is a squalene emulsion. In some embodiments, the adjuvant is a TLR4 immunostimulant (e.g., SLA, GLA), e.g., as described in Van Hoeven at al. PLoS One. 11(2):e0149610 (2016). In some embodiments, the adjuvant is a TLR7/8 immunostimulant (e.g., R848, IMQ, 3M-052), e.g., as described in Dowling D. ImmunoHorizons (6):185-197 (2018). In some embodiments, the adjuvant is a TLR9 immunostimulant (CpG), e.g., as described in Bode et al. Expert Rev Vaccines. 10(4):499-511 (2011). In some embodiments, the adjuvant is saponin (QS21), e.g., as described in Zhu et al. Nat Prod Chem Res. 3(4):e113 (2016).

In some embodiments, the vaccine comprises a combination of two or more adjuvants (e.g., squalene emulsion and alum or a TLR4 immunostimulant).

In some embodiment, the pharmaceutical composition includes chelating agent selected from BAPTA-AM (N,N′-[1,2-ethanediylbis(oxy-2,1-phenylene)]bis[N-[2-[(acetyloxy)methoxy]-2-oxoethyl]-1,1′-bis[(acetyloxy)methyl] ester-glycine)), EDTA (Ethylenediamine tetraacetic acid), and EGTA (aminopolycarboxylic acid).

### Combination Products

In another aspect, the disclosure provides a pharmaceutical composition that includes both a recombinant influenza neuraminidase (NA) tetramer, as described herein, and an influenza Hemagglutinin (HA) vaccine antigen and/or influenza M2e vaccine antigen. In embodiments, the influenza HA vaccine antigen is a protein nanostructure displaying influenza HA, or the ectodomain thereof, or another suitable antigenic fragment. Illustrative influenza HA vaccines antigens that may be combined with a tetramer of the present disclosure include, without limitation, soluble HA trimer, embryonated chicken egg-based influenza HA, live virus, live attenuated virus, inactivated virus, detergent purified, lipid-containing nanoparticle, mRNA vaccine, DNA vaccine, and adenoviral vector vaccine. These and other vaccine antigens suitable for combination with a tetramer described herein are described in WO 2013/044203, WO 2014/019990, WO 2009/050390, AU492446B2, WO 2008/021959, WO 2007/052163, WO 2010/077986, U.S. Pat. No. 8,951,537, WO 2022/150717, WO 2010/036948, and WO 2007/022151, the contents of which are incorporated by reference herein for purpose of describing such vaccine antigens.

In some embodiments, a pharmaceutical composition includes both a recombinant influenza neuraminidase NA1 tetramer, as described herein, and an influenza Hemagglutinin (HA) vaccine antigen. In some embodiments, a pharmaceutical composition includes both a recombinant influenza neuraminidase NA2 tetramer, as described herein, and an influenza Hemagglutinin (HA) vaccine antigen. In some embodiments, a pharmaceutical composition includes both a recombinant influenza neuraminidase B/Vic tetramer, as described herein, and an influenza Hemagglutinin (HA) vaccine antigen. In some embodiments, a pharmaceutical composition includes a combination of N1, N2, and B/Vic influenza virus neuraminidases, as described herein, and an influenza Hemagglutinin (HA) vaccine antigen.

In embodiments, the influenza HA vaccine antigen is a protein nanostructure displaying influenza HA, or the ectodomain thereof, or another suitable antigenic fragment. Illustrative protein nanostructures, in some cases described as protein-based Virus-Like Particles (pbVLPs), are provided in [U.S. Pat. Nos. 10,248,758, 9,630,994, 10,501,733, 11,771,755], the contents of which are incorporated by reference herein for purpose of describing such protein nanostructures or pbVLPs. In embodiments, the disclosure provides a pharmaceutical composition that includes both a recombinant influenza neuraminidase (NA) tetramer, as described herein, and a protein nanostructure that displays influenza HA, or an ectodomain thereof, or other antigenic fragment of influenza HA.

In some embodiments, the disclosure provides a pharmaceutical composition that includes both a recombinant influenza neuraminidase (NA) tetramer, as described herein, and a “cocktail” of two or more protein nanostructures each displaying an antigenic fragment of influenza HA, or an ectodomain thereof. In some embodiments, the disclosure provides a pharmaceutical composition that includes both a recombinant influenza neuraminidase (NA) tetramer, as described herein, and a “cocktail” of four protein nanostructures displaying i) H1, A/Wisconsin/588/2019; ii) H3, A/Darwin/6/2021; iii) B/Vic, B/Austria/1359417/2021; and iv) B/Yam, B/Phuket/3073/2013.

In some embodiments, the disclosure provides a pharmaceutical composition that includes both a recombinant influenza neuraminidase (NA) tetramer, as described herein, and a nanostructure comprising a “mosaic” of two or more influenza antigenic polypeptides co-displayed on the surface of the nanostructure. In some embodiments, the disclosure provides a pharmaceutical composition that includes both a recombinant influenza neuraminidase (NA) tetramer, as described herein, and a “mosaic” of influenza antigenic polypeptides i) H1, A/Wisconsin/588/2019; ii) H3, A/Darwin/6/2021; iii) B/Vic, B/Austria/1359417/2021; and iv) B/Yam, B/Phuket/3073/2013 co-displayed on the surface of the nanostructure.

The influenza HA ectodomain may be displayed on a variety of protein nanostructures. The protein nanostructures of the present invention may comprise multimeric protein assemblies adapted for display of molecules such as antigens (e.g., engineered ectodomains). The protein nanostructures, in some embodiments described herein, comprise at least a first component displaying an engineered ectodomain and, optionally, a second component. The engineered ectodomain may include one or more amino acid substitutions. The first component may comprise or consist of a fusion protein. In some embodiments, the fusion protein comprises an assembly domain having a protein sequence designed by computational methods to assemble to form a nanostructure. In some embodiments, the first component is a trimeric component in which the assembly domains form trimers related by 3-fold rotational symmetry, and/or the second component is a pentameric component, in which the assembly domains form pentamers related by 5-fold rotational symmetry. In some embodiments, the combination of the two components forms an “icosahedral particle” having 153 symmetry. Together these components may be arranged such that the members of each component are related to one another by symmetry operators. A general computational method for designing self-assembling protein materials, involving symmetrical docking of protein building blocks in a target symmetric architecture, is disclosed in Patent Pub. No. US 2015/0356240 A1.

The “core” of the protein nanostructure is used herein to describe the central portion of the protein nanostructure. For clarity, the term “core” as used herein excludes molecules displayed by the nanostructure. The core may serve to assemble multiple copies of the displayed molecule, such as an antigen (e.g., an engineered influenza HA ectodomain). Without being bound by theory, this may increase the immunogenicity of an antigen. The disclosure envisions nanostructures in which the core is either non-covalently associated with the displayed antigen; covalently linked to the display antigen (such as by chemical conjugation); or, in preferred embodiments, linked to the displayed antigen through a polypeptide linker in a fusion protein. In some embodiments, the fusion protein comprises a first polypeptide comprising an antigen (e.g., an influenza HA ectodomain), and a first assembly domain. In some embodiments, an antigen (e.g., an influenza HA ectodomain) is non-covalently or covalently linked to the assembly domain. For example, an antigen (e.g., an influenza HA ectodomain) may be fused to the first component and configured to bind a portion of the first component, or a chemical tag on the first component. For example, a streptavidin-biotin (or neuravidin-biotin) linker can be employed. Alternatively, various bioconjugate linkers may be used. In some embodiments of the present disclosure, the antigen comprises further polypeptide sequences in addition to influenza HA protein.

In some embodiments, three copies of an antigen (e.g., an influenza HA ectodomain) polypeptide are displayed on a 3-fold axis. Thus, the protein nanostructure is capable of displaying 60 monomeric antigen (e.g., an influenza HA ectodomain) polypeptides. In some embodiments, the protein nanostructure is adapted for display of up to 12, 24, or 60 monomers. In some embodiments, a component may comprise a polypeptide linked to diverse engineered ectodomains, such that the protein nanostructure displays different ectodomains on the same nanostructure. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more different ectodomains are displayed. Non-limiting illustrative protein nanostructure are provided in Bale et al. Science 353:389-94 (2016); Heinze et al. J. Phys. Chem B. 120:5945-5952 (2016); King et al. Nature 510:103-108 (2014); and King et al. Science 336:1171-71 (2012).

In some embodiments, the disclosure provides a composition or pharmaceutical composition, comprising one or more nanostructures. Each nanostructure comprises a first component and optionally one or more further components, which collectively are assembled to form the nanostructure. In some embodiments, the first component may comprise an influenza HA, a protein ectodomain thereof, or other antigenic fragment of influenza HA. In another aspect, the disclosure provides a nanostructure comprising a trimeric component comprising a helix-forming segment as disclosed herein. In some embodiments, the nanostructure is a two-component nanostructure comprising the first, trimeric component and a second, pentameric component. In some embodiments, the trimeric component comprises a fusion protein comprising an engineered ectodomain of an influenza HA polypeptide fused to an I53-50A polypeptide.

Patent Pub No. US 2015/0356240 A1 describes various methods for designing protein assemblies. As described in US Patent Pub No. US 2016/0122392 A1 and in International Patent Pub. No. WO 2014/124301 A1, the isolated polypeptides of SEQ ID NOs: 458-459 were designed for their ability to self-assemble in pairs to form protein nanostructures, such as icosahedral particles. The design involved design of suitable interface residues for each member of the polypeptide pair that can be assembled to form the protein nanostructures. The protein nanostructures so formed include symmetrically repeated, non-natural, non-covalent polypeptide-polypeptide interfaces that orient a first assembly domain and a second assembly domain into protein nanostructures, such as one with an icosahedral symmetry. Thus, in one embodiment a first assembly domain and second assembly domain of the component are selected from the group consisting of SEQ ID NOs: 458-459. In each case, an N-terminal methionine residue present in the full-length protein is included, but may be removed to make a fusion that is not included in the sequence.

In some embodiments, the nanostructure is a two-component nanostructure comprising
a first trimeric component, wherein the first trimeric component comprises an engineered ectodomain of an influenza HA polypeptide comprising any one of SEQ ID NOs: 452-455; and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 459), 153-50A ΔCys (SEQ ID NO: 458), or I53-dn5A (SEQ ID NO: 460); and/or a second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of SEQ ID NOs: 461-463.

In some embodiments, an I53-50A multimerization domain comprises interface residues at amino acid residue positions: 25, 29, 33, 54, and 57.

In some embodiments, an I53-50B multimerization domain comprises interface residues at amino acid residue positions: 24, 28, 36, 124, 125, 127, 128, 129, 131, 132, 133, 135, and 139.

Residue numbers in each illustrative polypeptide that were identified as present at the interface of resulting assembled protein nanostructures (i.e., “identified interface residues”). In various embodiments, a first assembly domain and second assembly domain comprise an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical over its length, and identical at least at 1, at least at 2, at least at 3, at least at 4, at least at 5, at least at 6, at least at 7, at least at 8, at least at 9, at least at 10, at least at 11, at least at 12, or at least at 13 identified interface positions (depending on the number of interface residues for a given polypeptide), to the amino acid sequence of a polypeptide selected from the group consisting of SEQ ID NOs: 458-463.

The present disclosure provides a protein nanostructure comprising a trimeric component comprising a trimer polypeptide and a pentameric component comprising a pentamer polypeptide, wherein the trimer polypeptide is a fusion protein comprising, in N- to C-terminal order, an influenza HA ectodomain, a polypeptide linker, and an assembly domain.

In some embodiments, the assembly domain comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 458-460.

In some embodiments, the influenza HA ectodomain comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 452-455, or 465.

In some embodiments, the pentamer protein comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 461-463.

In some embodiments, the trimer protein comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 448-451, or 464.

In some embodiments, the polypeptide linker comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 456.

In some embodiments, the polypeptide linker comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 457.

In embodiments, the protein nanostructure is an I3-01 nanostructure. In some embodiments, the multimerization domain is a ferritin polypeptide.

In some embodiments, polynucleotides (e.g., mRNA) encoding protein nanostructures including a component comprising a viral protein monomer of a trimeric viral antigen are formulated in a delivery vehicle. In some embodiments, the delivery vehicle is a non-viral vector. In some embodiments, the delivery vehicle is a lipid nanoparticle (LNP). In some embodiments, the delivery vehicle is a liposome. In some embodiments, the delivery vehicle is a polymeric-non-viral vector, such as spermine, Polyethylenimine, chitosan, or polyurethane. In some embodiments, the delivery vehicle is a polymer delivery system, such as poly-amido-amine (PAA), poly-beta aminoesters (PBAEs) or polyethylenimine (PEI). In some embodiments, the delivery vehicle is a ferritin nanoparticle. In some embodiments, the delivery vehicle is an encapsulin.

In some embodiments, polynucleotides (e.g., mRNA) encoding protein nanostructures including a component comprising a viral protein monomer of a trimeric viral antigen are formulated in a nanoparticle. In some embodiments, the nanoparticle is a lipid nanoparticle (LNP). In some embodiments, the polynucleotides are formulated in a lipid-polycation complex, referred to as a cationic LNP. As a non-limiting example, the polycation may include a cationic peptide or a polypeptide such as, but not limited to, polylysine, polyornithine and/or polyarginine. In some embodiments, the polynucleotides are formulated in a LNP that includes a non-cationic lipid such as, but not limited to, cholesterol or dioleoyl phosphatidylethanolamine (DOPE).

In some embodiments, the nanostructure is assembled from a mixture of first components, for example, the protein nanostructure may co-displaying one or more copies of two or more influenza antigenic polypeptides on each nanostructure (termed herein, a “mosaic” nanostructure). In variations, the disclosure provides a “mosaic” nanostructure generated by mixing trimeric components comprising influenza antigenic polypeptides before assembly into nanostructures, so that each nanostructure contains components displaying one influenza HA antigenic polypeptide and components displaying a different influenza HA antigenic polypeptide. Accordingly, the disclosure further provides a nanostructure comprising a plurality of first components, some first components comprising an H1, A/Wisconsin/588/2019 antigenic polypeptide, some first components comprising an H3, A/Darwin/6/2021 antigenic polypeptide, some first components comprising a B/Vic, B/Austria/1359417/2021 antigenic polypeptide, and some first components comprising a B/Yam, B/Phuket/3073/2013 antigenic polypeptide.

In some embodiments, the nanostructure composition comprises two or more separate nanostructures, for example, each nanostructure may display one or more copies of a single influenza antigenic polypeptide on its surface (termed herein, a “cocktail” nanostructure).

### Methods

### Method of Immunizing

In another aspect, the disclosure provides a method of immunizing a subject against infection by influenza, the method comprising administering to the subject a composition of the disclosure.

In some embodiments, the composition is administered by subcutaneous injection. In some embodiments, the composition is administered by intramuscular injection. In some embodiments, the composition is administered by intradermal injection. In some embodiments, the composition is administered by intranasally. In some embodiments, the composition is administered by intravenously.

The disclosure provides vaccines comprising the nanostructures described herein. Such compositions can be used to raise antibodies in a mammal (e.g., a human). The vaccine compositions of the disclosure typically include a pharmaceutically acceptable carrier, and a thorough discussion of such carriers is available in Remington: The Science and Practice of Pharmacy.

Vaccines of the disclosure may be prophylactic (i.e., to prevent disease) or therapeutic (i.e. to reduce or eliminate the symptoms of a disease). The term prophylactic may be considered as reducing the severity of or preventing the onset of a particular condition. For the avoidance of doubt, the term prophylactic vaccine may also refer to vaccines that ameliorate the effects of a future infection, for example by reducing the severity or duration of such an infection.

In one aspect, the disclosure provides a method of immunizing a subject against infection by influenza, the method comprising administering the vaccine described herein. In some embodiments, a vaccine comprises a recombinant influenza neuraminidase (NA) tetramer and a nanostructure comprising two or more influenza HA antigenic polypeptides co-displayed on the surface of the nanostructure. In some embodiments, a vaccine comprises a recombinant influenza neuraminidase (NA) tetramer and two or more nanostructures each displaying an antigenic fragment of influenza HA, or an ectodomain thereof.

In some embodiments, a vaccine comprising a recombinant influenza neuraminidase (NA) tetramer and a nanostructure comprising four co-displayed influenza HA antigenic polypeptides raises antibodies in a mammal against both HA and NA antigenic polypeptides. In some embodiments, a vaccine comprising a recombinant influenza neuraminidase (NA) tetramer and a nanostructure comprising four co-displayed influenza HA antigenic polypeptides generates immunogenicity against both HA and NA antigenic polypeptides.

Method of Treating and/or Preventing Influenza

In another aspect, the disclosure provides a method of treating and/or preventing influenza in a subject in need thereof, the method comprising administering to the subject a composition of the disclosure. In some embodiments, the method generates an antibody response to the tetramer at least as effectively as administering a tetramer with a tetrabrachion tetramerization domain.

In one aspect, the disclosure provides a method of immunizing a subject against infection by influenza, the method comprising administering the vaccine described herein. In some embodiments, a vaccine comprises a recombinant influenza neuraminidase (NA) tetramer and a nanostructure comprising two or more influenza HA antigenic polypeptides co-displayed on the surface of the nanostructure. In some embodiments, a vaccine comprises a recombinant influenza neuraminidase (NA) tetramer and two or more nanostructures each displaying an antigenic fragment of influenza HA, or an ectodomain thereof.

### Kits

In another aspect, the disclosure provides a kit including a composition of the disclosure and instructions for use.

### Method of Producing a Recombinant Influenza Neuraminidase (NA) Tetramer

In another aspect, the disclosure provides a host cell that have been transfected or transduced with a recombinant expression vectors disclosed herein, wherein the host cells can be either prokaryotic or eukaryotic. The cells can be transiently or stably transfected or transduced. Such transfection or transduction of expression vectors into prokaryotic and eukaryotic cells can be accomplished via any technique known in the art, including but not limited to standard bacterial transformations, calcium phosphate co precipitation, electroporation, or liposome mediated-, DEAE dextran mediated-, polycationic mediated-, or viral mediated transfection. (See, for example, Molecular Cloning: A Laboratory Manual (Sambrook, et al, 1989, Cold Spring Harbor Laboratory Press; Culture of Animal Cells: A Manual of Basic Technique, 2nd Ed. (R. I. Freshney. 1987. Liss, Inc. New York, NY). comprising the polynucleotide described herein.

In some embodiments, the host cells include E. coli cells, 293 and 293F cells, HEK293 cells, Sf9 cells, Chinese hamster ovary (CHO) cells or any other cell line used in the production of recombinant proteins.

In some embodiments, the disclosure provides recombinant expression vectors comprising the isolated nucleic acid of any embodiment or combination of embodiments of the disclosure operatively linked a suitable control sequence. “Recombinant expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In embodiments, the expression vector comprises a plasmid. However, the disclosure is intended to include other expression vectors that serve equivalent functions, such as viral vectors.

In another aspect, the disclosure describes a method of producing a recombinant influenza neuraminidase (NA) tetramer, comprising culturing the host cell of the disclosure in cell culture media, and harvesting the tetramer from the cell culture media or from the cells. In some embodiments, the method produces an increased yield of tetramer compared to production of a tetramer with a tetrabrachion tetramerization domain.

In some embodiment, the methods include chelating agent selected from BAPTA-AM (N,N′-[1,2-ethanediylbis(oxy-2,1-phenylene)]bis[N-[2-[(acetyloxy)methoxy]-2-oxoethyl]-1,1′-bis[(acetyloxy)methyl] ester-glycine)), EDTA (Ethylenediamine tetraacetic acid), and EGTA (aminopolycarboxylic acid).

### EXAMPLES

### Materials and Methods

Protein BLAST search. Protein-based BLAST searches were performed on the tetrabranchion (TB) sequence from PDB 1FE6 using tools available on NCBI (see, the World Wide Web at blast.ncbi.nlm.nih.gov/Blast.cgi).

Structural modeling. PyMol version 2.5.2 was used to analyze all structural models and generate images. To generate a homology model of DA4B based on the structure of TB, Rosetta was used to introduce DA4B-based mutations into the structure of PDB 1FE6 using a resfile that matched the sequence of DA4B. C4 symmetry was maintained, and two rounds of packing was performed using default settings. Structure-based prediction using Colabfold (Ref 13) was performed with default settings using four identical copies of the DA4B sequence.

HEK293 expression. One day prior to transfections, Expi293F cells (Thermo Fisher Scientific) were counted and seeded at 2.5×10⁶ cells/mL and subsequently incubated overnight at 36° C. with shaking at 120 rpm. The next day the cells were counted and diluted to 3×10⁶ cells/mL. Cells were then transiently transfected with the desired plasmid according to manufacturer directions (Thermo Fisher Scientific), with the exception that Transporter 5 (Polysciences) was used as the transfection reagent at a ratio of 3.0 μg of Transporter 5 per 1 μg of plasmid DNA. Transfected cells were then incubated at 36° C. for 4 days while shaking at 120 rpm.

Western blots. Western Blots were performed against HEK293 cell supernatants after small-scale expression using Anti-His HRP conjugated antibody. Supernatant was diluted 2-fold in 2× Laemmli loading buffer (Bio-Rad) with 5% (v/v) β-mercaptoethanol, then heated to 95° C. for 10 minutes. 10 μl was loaded onto a NuPAGE 4-12% Bis-Tris protein gel along with 7 μl of His-tagged protein standard/ladder (Invitrogen, #LC5606), and ran for 30 minutes at 200V. Proteins were transferred onto an Immuno-Blot PDVF membrane (Bio-Rad). Following transfer, the blot was blocked with 2% milk (w/v) in 1×PBS, 0.05% Tween 20 for 1 hour at room temperature with rocking. Anti-His HRP conjugated antibody (R&D systems MAB050H) was diluted 1:7000 in 2% milk (w/v) in 1×PBS, 0.05% Tween 20 and incubated with the membrane for 1 hour at room temperature with rocking. The membrane was then washed three times for five minutes using 1×PBS, 0.05% Tween 20, and His-tagged protein was detected using (BioFx TMB Enhanced One Component HRP Membrane Substrate, Surmodics) and captured on BioRad ChemiDoc MP imaging system.

IMAC purification. NA protein samples were purified directly from HEK Expi293F cell supernatant by IMAC. Ni²⁺-Agarose resin (Cube Biotech, Indigo) was washed with 5 column volumes (CV) of water followed by 5 CV of equilibration buffer (20 mM Tris pH 8.0, 250 mM NaCl, 5% glycerol, 30 mM imidazole), then resuspended in 1 CV of equilibration buffer. Supernatants were clarified by centrifugation at 4,000×g followed by filtration using 0.2 mm vacuum filtration units. Resin suspension was added to each supernatant at a ratio of 4 μL of resin per mL of supernatant. The supernatant-resin slurries were incubated with gentle rocking at 4° C. for ~16-18 hours, then the resin was collected by application to empty gravity columns (BioRad, 7321010). The resin beds were washed with 20 CV equilibration buffer, and proteins were eluted using 6 CV elution buffer (20 mM Tris pH 8.0, 250 mM NaCl, 5% glycerol, 300 mM imidazole) in three 2 CV increments with 30-minute incubations between each application.

SEC purification and assembly of HA-VLP nanostructure. Each IMAC for NA protein samples elution was injected onto either a Superdex 200 Increase 10/300 GL column or a Superose 6 Increase 10/300 GL column (Cytiva) equilibrated in SEC running buffer (20 mM Tris pH 8.0, 250 mM NaCl, 5% glycerol) and eluted with 1.2 CV of SEC running buffer to further purify the antigens by size exclusion chromatography (SEC). Following analysis, purified NA protein samples were supplemented with 1 mM CaCl₂ prior to flash freezing in liquid nitrogen and storage at −80° C. Integration of peaks for tetrameric species was measured using Unicorn 7.6 software (Cytiva) with default settings. HA-CompA samples from H1, H3, B/Vic and B/Yam HA antigens (SEQ ID NOs: 487, 489, 491, 493) were assembled into the mosaic HA-VLP nanostructure sample by mixing with the complementary “CompB” sequence (SEQ ID NO: 495, Ref 16) as described in Ref 15 for equivalent trimeric and pentameric components, and further purifying by SEC and a Superose 6 Increase 10/300 GL column (Cytiva).

Catalytic activity measurements. Catalytic activity of purified NA samples was assessed using the NA-Fluor™ Influenza Neuraminidase Assay Kit from Thermo Fisher Scientific (Catalog number 4457091). A day prior to performing the assay, 40% ethanol was added to the stop solution (Applied Biosystems, 4457090) and NA-Fluor substrate (Thermo Fisher Scientific, 4457089) was reconstituted in 5 mL of Milli-Q water. On the day of running the assay, 2× Assay Buffer (Applied Biosystems, 4457088) was diluted to 1× with Milli-Q water. 50 μL of 1× Assay Buffer was dispensed into all wells of a black 96 well plate (Greiner, 655209). NA constructs were normalized to 0.1 or 0.5 μM, and 50 μL of each sample was loaded into the first column of the black 96 well plate. A multi-channel was used to mix and transfer 50 μL from the first column to the second, and from there a serial dilution performed to column eleven, at which point 50 μL was discarded leaving only 1× assay buffer in column twelve. Next, while in a darkened room, 50 μL of 200 μM NA-Fluor was added to each well and the plate was shaken to mix thoroughly. The plate was then covered and placed in a dark incubation chamber set to 37° C. for one hour. After hour long incubation, 100 μL of stop solution was added to terminate the reaction of NA-Fluor. Fluorescence was then read from the top of the plate using an excitation wavelength at 350 nm and an emission wavelength at 440 nm using a Biotek Synergy H1 microplate reader.

Biolayer interferometry (BLI) assay. Biotinylated oseltamivir (synthesized according to Ref 7) and CD6 antibody (Kerafast, #EFD001) were respectively normalized in concentration to 0.05 μg/mL and 10 μg/mL in BLI assay buffer (PBS, 0.5% BSA, 0.05% Tween 20, pH 7.4) in a sufficient volume to load 200 μL per well of a black 96-well microplate (Greiner, 655209). Briefly, on an Octet Red96e instrument, pre-hydrated Streptavidin biosensors (Sartorius, 18-5019) or Protein G biosensors (Sartorius, 18-5022) were dipped into assay buffer for 60 seconds to achieve a baseline, with Streptavidin biosensors used for biotinylated oseltamivir and Protein G biosensors used for CD6 antibody. Next, the biosensors were dipped into biotinylated oseltamivir or CD6 antibody for 60 seconds or 120 seconds respectively for immobilization, followed by an additional baseline step. The immobilized oseltamivir was allowed to associate with 200 μL of purified NA antigens (at 10 μg/mL in BLI assay buffer supplemented with either 1 mM CaCl₂ or 5 mM EDTA) or cell supernatants for 120 to 700 seconds, and then the biosensors were dipped back into assay buffer for 120 to 700 seconds to observe any possible dissociation. Shift measurements over time were corrected by subtracting measurements against a negative sample that did not contain NA antigens. The difference between the shift measurement at 100 seconds and 0 seconds is reported in all tables unless otherwise noted.

NanoDSF using intrinsic protein fluorescence (ITF). Protein thermal stability and fluorescent baseline measurements were determined using ITF on a nanoDSF instrument (UNchained Laboratories, UNcle). Antigen concentrations were normalized to 0.107-0.3 mg/mL using sample buffer (20 mM Tris pH 8.0, 250 mM NaCl, 5% glycerol) supplemented either CaCl₂) or EDTA. In all cases the concentrations of these additions were 1 mM CaCl₂) or 5 mM EDTA, with the exception of N2-based samples which used 5 mM CaCl₂) or 1 mM EDTA. Each sample was applied to a quartz capillary cassette (UNchained Laboratories, UNi) for ITF measurements over a temperature ramp (15-95° C., 0.5-1° C./min) after a 5-minute equilibration at 15° C. Melting temperatures were determined for representative single samples from barycentric mean calculations using Uncle Analysis V6.0 software with default settings. Baseline fluorescence near 25° C. was measured from barycentric mean calculations based on two to three replicates by averaging points that were collected at temperatures closest to 25° C. for each replicate.

NanoDSF using SYPRO. Thermal melt profiles NA constructs were determined by normalizing sample concentrations to 0.3-0.4 mg/mL in sample buffer (20 mM Tris, 250 mM NaCl and 5% Glycerol pH 8.0) supplemented with either 1 mM CaCl₂) or 5 mM EDTA. SYPRO Orange Protein Gel Stain (Invitrogen S6651) was then diluted 1:20 in each sample buffer and 3.5 μL was added to 31.5 μL of normalized NA constructs. Each sample was applied to a quartz capillary cassette (UNchained Laboratories, UNi) for ITF measurements over a temperature ramp (15-95° C., 0.5-1° C./min) after a 5-minute equilibration at 15° C. Melting temperatures were determined using fluorescent area measurements from representative single samples using Uncle Analysis V6.0 software with default settings.

Animal immunizations. For all studies that only included NA antigens, female BALB/c mice were immunized intramuscularly on days 0 or 21 with 1 μg of purified NA protein mixed 1:1 with Addavax™ adjuvant. For the study involving combinations of an NA antigen with the HA VLP nanostructure, female BALB/c mice were immunized intramuscularly on days 0 or 21 with 0.1 μg, 1 μg, or 4 μg of purified NA protein with or without 0.1 μg of HA VLP nanostructure (with protein amount referring to total HA protein), with final formulations mixed 1:1 with Addavax™ adjuvant. Serum samples were collected on day 0 (prior to first immunization), day 21 (prior to second immunization), and day 35. Day 0 serum samples were pooled into smaller sets of samples (“Pre-Bleed Pool Day 0”) for ELLA analysis.

ELLA assay. All work involving ELLA assays and calculations of IC50 titers was performed using live native influenza viruses. To determine the titer of virus used in the assay, supernatant containing influenza virus (either A/Darwin/6/2021, A/Michigan/45/2015 or B/Colorado/06/2017) grown in MDCK cells (ATCC, #CCL-34) was serially diluted 2-fold in sample diluent (PBS containing bovine serum albumin [Thermo Fisher Scientific #10082-147] and Tween 20) and was transferred to a fetuin (Sigma-Aldrich, #F2379) coated plate (Thermo Fisher Scientific #442404), which was then placed in a humidified incubator overnight. The day after incubation, the plate was washed and incubated with a solution of Arachis hypogaea Lectin (PNA) conjugated with Horseradish Peroxidase (HRPO) (Sigma-Aldrich, #L7759-1MG). Then, to the washed plate a chromogenic peroxidase substrate (Sigma-Aldrich, #P8287) was added to develop a colorimetric reaction, which was later stopped by sulfuric acid (Sigma-Aldrich, #258105). Optical density (OD) results from the colorimetric reaction were measured and plotted versus the dilution, and an optimal dilution was identified at 90% of the maximum signal. To measure ELLA titers in serum from immunized mice, serum samples were heat-inactivated for 30 minutes at 56° C., then serially diluted 2-fold in fetuin coated plates mixed with an equal volume of supernatant containing influenza virus at an optimal dilution and placed in a humidified incubator overnight at 37° C. Duplicate runs for each sample were performed in the same plate, with negative control wells (no added virus) and positive control wells (no added serum) included in all plates. The day after incubation, the plates were washed and incubated with PNA-HRPO solution. Then, to the washed plates a chromogenic peroxidase substrate was added to develop a colorimetric reaction, which was later stopped by sulfuric acid. The ELLA IC50 titer of each sample run corresponds to the reciprocal highest sample dilution that results in at least 50% inhibition of the maximum signal as measured by OD.

### Example 1: DA4B Tetramerization Domain

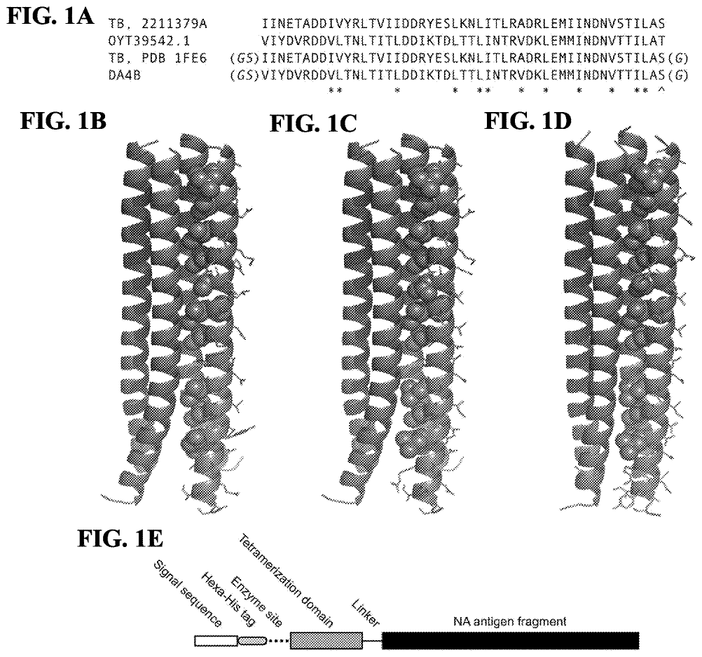

A Basic Local Alignment Search Tool (BLAST) search was performed against all publicly available sequences from the NIH National Center for Biotechnology Information (NCBI) using as query an alpha-helical segment of the tetrabrachion protein (Genbank accession 2211379A, residues 1238-1286, referred to here as “TB”). A segment of a hypothetical protein (Genbank accession OYT39542.1, residues 1348-1396) from the hyperthermophilic archaebacterium Desulfurococcales archaeon was identified as having 49% sequence identity to TB (FIG. 1A). To reliably compare this sequence with a published TB sequence used in a crystal structure (PDB 1FE6), the terminal ends of this sequence were modified to match the sequence provided in PDB 1FE6 by adding a “Gly-Ser” segment at the N-terminus and an additional glycine at the C-terminus. Further, to improve the chance of this sequence supporting successful genetic fusion to NA antigens through its C-terminus, a sequence was generated in which threonine residue near the C-terminal end was mutated to serine (T51S), as the TB tetramerization domain natively contains a serine in this position. This sequence was named “DA4B”.

Computational modeling was used to investigate whether DA4B has potential to form C4-symmetric tetrameric complexes which could be genetically fused to NA. First, a homology model was generated from an existing crystal structure of TB (PDB 1FE6) using symmetric modeling in Rosetta macromolecular modeling software. The generated model showed that residues involved in intermolecular hydrophobic packing were strongly conserved between TB and DA4B (FIGS. 1B and 1C), and that no clear steric clashes were introduced by threading the DA4B sequence onto the TB structure. Surface-exposed residues were more often less conserved between the two structures. Similar results were obtained through protein structure predictions using Colabfold, which generated nearly-identical C4-symmetric structures, with subtly different backbone positioning shown for the N-terminus (FIGS. 1B and 1D).

### Example 2: Naming of Constructs

Constructs were descriptively named based on features of the designs. Each name starts with the tetramerization domain used and is proceeded by name tags that describe the linker used and the NA head antigen, all separated by underscores. As examples:
TB_L2_70-N1-MI15: TB genetically fused by a 2-residue linker to an N1-MI15 antigen fragment that starts at residue 70, with no mutations added within the NA fragment.
DA4B_L8_83-N2-DA21: DA4B genetically fused by an 8-residue linker to an N2-DA21 antigen fragment that starts at residue 83, with no mutations added within the NA fragment.
DA4B-del3_L10_83-N1-WI19-11mut: DA4B with N-terminal deletion of 3 residues genetically fused by a 10-residue linker to an N1-WI19 antigen fragment that contains the “11mut” set of stabilizing mutations.

### Example 3: Experimental Comparison Between TB and DA4B for NAs from Seasonal Subtypes

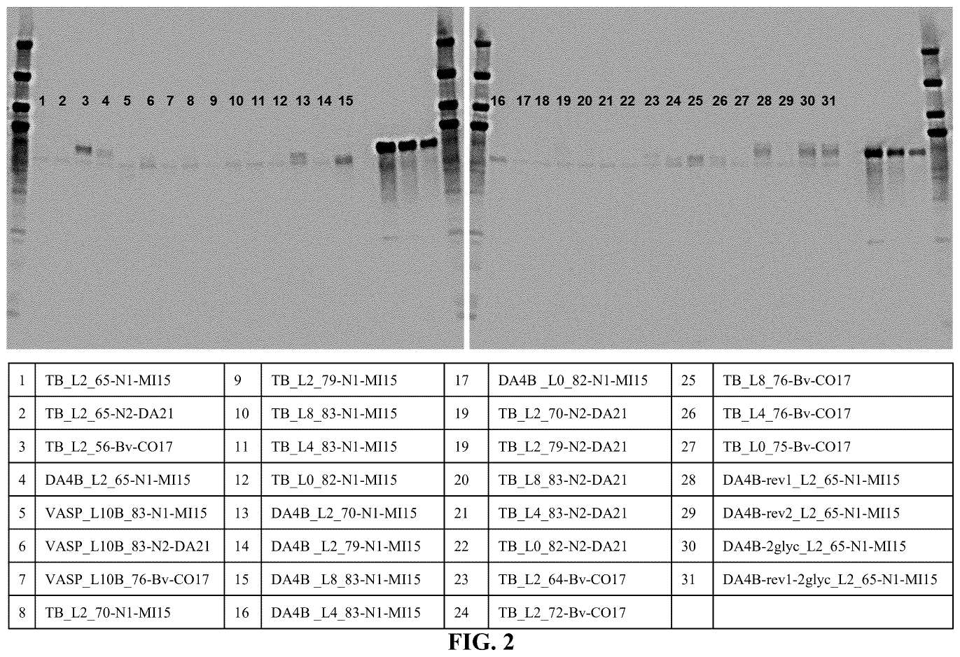

In a first experiment, a variety of designs were expressed at small-scale for representative N1, N2 and By NA antigens. TB was tested for all subtypes, while DA4B was only tested for N1 constructs. Alternate versions of DA4B were also minimally tested that altered select surface and buried residues on the tetramerization domain. Within each subtype, all constructs tested different connections between the tetramerization domains and NA antigens based on the linker used and the start point of the NA head domain. Secretion levels were analyzed from supernatants by western blots, which revealed differences in yield that appeared related to NA strain, connection, and tetramerization domain, with many designs not secreting at detectable levels by the western blot analysis. When comparing equivalent designs that used either TB or DA4B, a surprising trend was seen that suggested consistent higher yields from DA4B-based constructs. For example, as shown in FIG. 2, DA4B_L2_65-N1-MI15, DA4B_L2_70-N1-MI15 and DA4B_L8_83-N1-MI15 showed clearly higher signals respectively than TB_L2_65-N1-M115, TB_L2_70-N1-MI15 and TB_L8_83-N1-MI15. As well, compared to DA4B_L2_65-N1-MI15, equivalent versions of this design that further modified DA4B showed even higher yields. Finally, yields were also elevated for DA4B-based N1 designs compared to a control that was made using the human vasodilator stimulated phosphoprotein (VASP) tetramerization domain, which is also commonly studied in the field (Ref 4, 10, 12).

Based on this, representative sequences of NA antigens from N1 (A/Michigan/45/2015, “N1-MI15”), N2 (A/Darwin/6/2021, “N2-DA21”), B/Victoria (B/Colorado/06/2017, “By-CO17”) and B/Yamagata (B/Phuket/3073/2013, “By-PH13”) that were genetically fused to either DA4B or TB were chosen for scale-up to compare properties of subsequent soluble antigens. Novel constructs were designed for N2, By and By soluble tetramers to enable this analysis. The genetic fusions for N1-MI15 used a two-residue flexible linker between either tetramerization domain and the NA head domain from residues 70-469 that has previously been studied in literature² (DA4B_L2_70-N1-MI15 and TB_L2_70-N1-MI15). All others used an 8-residue flexible linker and NA head domains of residues 83-469 (N2-DA21, with constructs named DA4B_L8_83-N2-DA21 and TB_L8_83-N2-DA21) or 76-466 (Bv-CO17 and By-PH13, with constructs named DA4B_L8_83-Bv-CO17, TB_L8_83-Bv-CO17, DA4B_L8_83-Bv-PH13 and TB_L8_83-Bv-PH13). All sequences were preceded by a signal peptide, a hexa-histidine tag and an enzymatic cleavage tag to allow for secretion of tagged proteins (FIG. 1E).

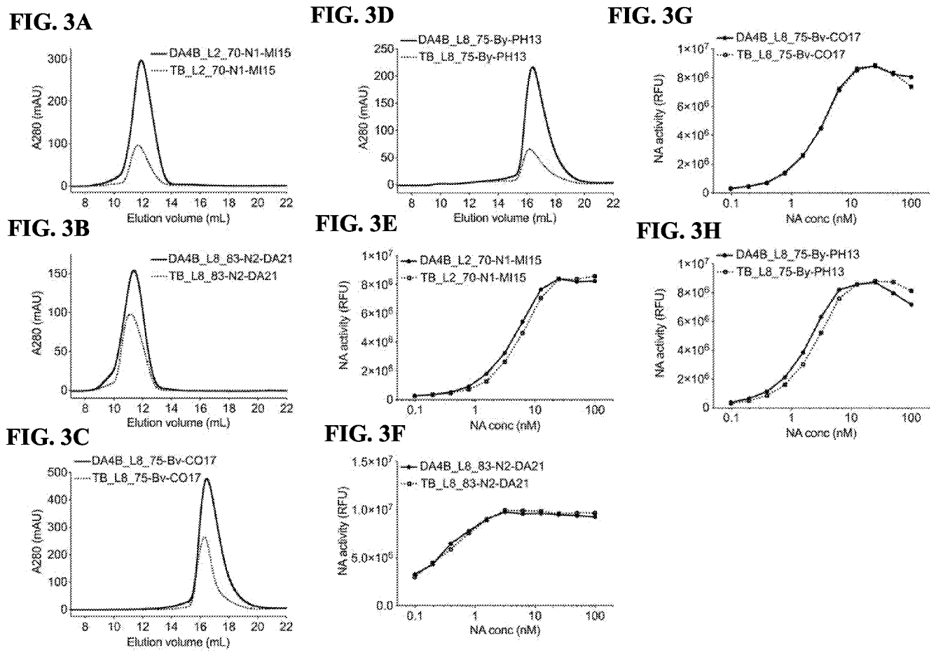

After secretion from HEK293 cells and immobilized metal affinity (IMAC) purification, size-exclusion chromatography (SEC) purification showed monodisperse species that were consistent with tetrameric NA antigens (FIGS. 3A-3D). SEC peaks were consistently higher for DA4B3-based antigens than TB-based antigens, with final SEC yields seen to be 1.6-3.4× higher for each DA41B-based antigen (Table 7). SEC profiles indicated that all proteins were well-formed tetramers. As shown in Table 7, integration of SEC peaks from initial DA41B- and TB-based constructs, showing improved yields from constructs containing DA41B over equivalent designs that contain TB regardless of the antigen subtype used.

TABLE 7
Integration of SEC peaks to quantify yields:

| | |
|---|---|
| Sample | Peak integration (mL*mAU) |

N1

| | |
|---|---|
| TB_L2_70-N1-MI15 | 153.2 |
| DA4B_L2_70-N1-MI15 | 523.9 |

N2

| | |
|---|---|
| TB_L8_83-N2-DA21 | 172.8 |
| DA4B_L8_83-N2-DA21 | 281.2 |

Bv

| | |
|---|---|
| TB_L8_75-Bv-CO17 | 385.5 |
| DA4B_L8_75-Bv-CO17 | 881.1 |

By

| | |
|---|---|
| TB_L8_75-By-PH13 | 164.5 |
| DA4B_L8_75-By-PH13 | 444 |

The catalytic active site of NA is a useful target for antibodies which can inhibit viral replications, with catalytic activity⁶ or binding to surface-immobilized biotinylated small molecule inhibitors⁷ or antibodies capable of informing proper foldedness of this key epitope. Catalytic activity was compared within purified antigens of each strain, showing activity levels that were similar within each subtype/lineage (FIGS. 3E-H). As well, binding to oseltamivir was maintained for all antigens, with all DA4B3-based samples showing similar or elevated binding compared with TB-based counterparts. Table 8 shows binding to oseltamivir from various DA4B- and TB-based constructs using N1, N2 and B NAs, as measured by BLI. BLI shift values were measured 100s into the association phase of soluble NA antigens immobilized oseltamivir on BLI tips. These data show that DA4B soluble tetramers demonstrate similar or improved binding to oseltamivir compared to equivalent TB-based constructs. Further, these data show that modifications to the connection between N1 NA antigens and DA4B can improve binding to oseltamivir. As well, these data show that addition of stabilizing mutations to the N1 NA head can improve binding to oseltamivir. Improved binding to oseltamivir suggests improved structuring of the catalytic active site of NA, which is a useful epitope targeted by protective antibodies. Thermal denaturations for these initial constructs was monitored within each strain using intrinsic tryptophan fluorescence (ITF), showing similar melting temperatures and fluorescence baseline shifts within antigens from the same strains. As shown in Table 9, ITF-based melting temperatures and fluorescence baselines from multiple DA4B- and TB-based designs. All data was collected in the presence of calcium chloride or EDTA, with fluorescence baselines only calculated in the presence of calcium chloride at 25° C. These data show that DA4B soluble tetramers demonstrate similar thermal stability compared to equivalent TB-based constructs. Further, these data show that modifications to the connection between N1 NA antigens and DA4B can improve thermal stability and cause fluorescence signals to become blue-shifted, which suggests improved structuring of the antigen. As well, these data show that addition of stabilizing mutations to the N1 NA head can improve thermal stability and cause fluorescence signals to become blue-shifted. Slightly higher fluorescence wavelengths were observed for DA4B-based constructs than TB-based constructs, which is likely influenced by differences in numbers of tyrosine residues between the two tetramerization domains. In all, for representative strains from each major seasonal subtype/lineage, genetic fusion of NA antigens to DA4B provides proteins with similar foldedness relative to identical antigens genetically fused to TB. Further, yields of secreted antigens were consistently higher for DA4B-based antigens.

TABLE 8
Binding of select N1 constructs to oseltamivir using BLI

| | | |
|---|---|---|
| | Shift (nm), | Shift (nm), |
| Sample | CaCl2 | EDTA |

N1

| | | |
|---|---|---|
| TB_L2_70-N1-MI15 | 1.56858091 | 0.90872946 |
| DA4B_L2_70-N1-MI15 | 1.32466263 | 0.86504059 |
| DA4B_L8_83-N1-MI15 | 3.04103021 | 1.65597254 |
| DA4B_L6_83-N1-MI15 | 3.08653773 | 2.22900438 |
| DA4B_L10_83-N1-MI15 | 2.94030079 | 0.88966376 |
| DA4B_L2_70-N1-MI15-6mutB | 2.90198644 | 1.02753858 |

N2

| | | |
|---|---|---|
| TB_L8_83-N2-DA21 | 1.48605255 | 1.6280046 |
| DA4B_L8_83-N2-DA21 | 2.25089323 | 1.74581974 |
| DA4B_L10_83-N2-DA21 | 2.31548454 | 1.92454009 |

Bv

| | | |
|---|---|---|
| TB_L8_75-Bv-CO17 | 3.1251449 | Not measured |
| DA4B_L8_75-Bv-CO17 | 3.38724777 | Not measured |
| DA4B_L8A_75-Bv-CO17 | 3.18686735 | Not measured |

By

| | | |
|---|---|---|
| TB_L8_75-By-PH13 | 1.34525 | Not measured |
| DA4B_L8_75-By-PH13 | 2.1650776 | Not measured |
| DA4B_L8A_75-By-PH13 | 2.2768166 | Not measured |

TABLE 9
ITF thermal denaturation and baseline measurements
for select N1, N2 and B NA constructs:

| | Melting | | Baseline |
|---|---|---|---|
| | temperatures | | near 25 C. |
| Sample | CaCl2 | EDTA | (CaCl2) |

N1

| | | | |
|---|---|---|---|
| TB_L2_70-N1-MI15 | 62.4 | 41.6 | 349.6 |
| DA4B_L2_70-N1-MI15 | 62.2 | 41.6 | 350.3 |
| DA4B_L8_83-N1-MI15 | 62.2 | 43.4 | 347.5 |
| DA4B_L6_83-N1-MI15 | 62.3 | 45.2 | 347.4 |
| DA4B_L10_83-N1-MI15 | 63.4 | 42.8 | 349.4 |
| DA4B_L2_70-N1-MI15-6mutB | 62.3 | Not measurable | 348.2 |

N2

| | | | |
|---|---|---|---|
| TB_L8_83-N2-DA21 | 56.2 | 53.4 | 347.3 |
| DA4B_L8_83-N2-DA21 | 56.2 | 53.2 | 347.9 |
| DA4B_L10_83-N2-DA21 | 56.0 | 53.2 | 348.0 |

Bv

| | | | |
|---|---|---|---|
| TB_L8_75-Bv-CO17 | 51.2 | 42.6 | 347.7 |
| DA4B_L8_75-Bv-CO17 | 51.0 | 42.5 | 348.1 |
| DA4B_L8A_75-Bv-CO17 | 51.2 | 42.4 | 348.4 |

By

| | | | |
|---|---|---|---|
| TB_L8_75-By-PH13 | 52.9 | 45.2 | 347.3 |
| DA4B_L8_75-By-PH13 | 53.2 | 46.0 | 347.0 |
| DA4B_L8A_75-By-PH13 | 53.1 | 45.6 | 347.3 |

### Example 4: Properties of Designed Connections Between DA4B and NA Antigens

Previous research suggests that different connections between tetramerization domains can impact various properties of NA antigens, including catalytic activity, antigenicity, thermal stability, and immunogenicity⁹. While the globular NA head domain (residues 83-469 for N1 and N2, or 76-466 for B NAs) is clearly structured, the “stalk” region preceding the head is considered less structured and can be partially or completely removed when making soluble recombinant NA antigens⁸. Because of these structural details, the antigenic and biophysical properties that derive from any connection between a tetramerization domain and NA head domain is influenced by the starting point of the antigen fragment, and optionally the composition of exogenous linker sequences inserted between the tetramerization domain and antigen fragment (FIG. 1E). Towards optimizing the specific connection between DA4B and different seasonal NA head domains, multiple combinations of antigen fragment start points and flexible exogenous linker sequences were considered separately for N1, N2, By and By antigens.

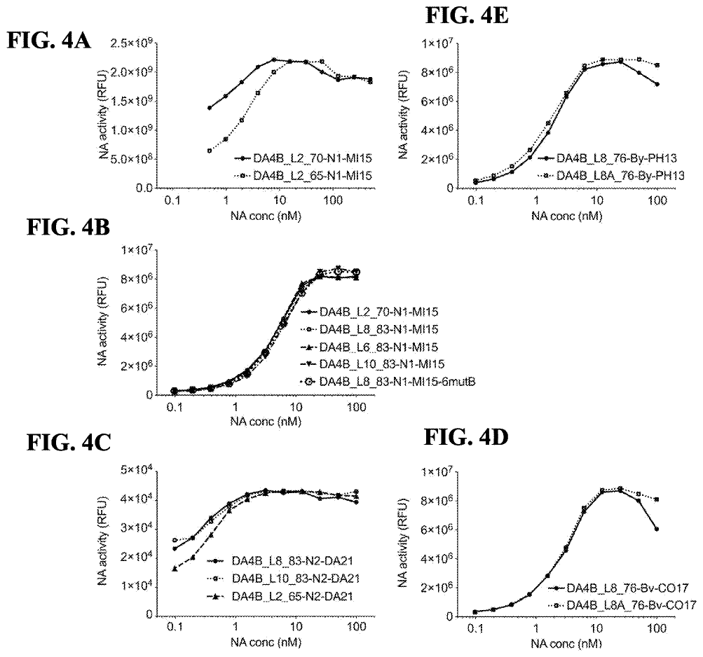

Two different connections were first further considered for N1-M15: DA4B_L2_70-N1-MI15 and DA4B_L2_65-N1-MI15, both of which use a two-residue flexible linker in between DA4B and the antigen fragment but use different start points for the antigen fragment. Protein for both was expressed, purified, and assessed for catalytic activity, with significantly higher catalytic activity shown for DA4B_L2_70-N1-MI15 (FIG. 4A), suggesting that a shorter connection between DA4B and the NA head domain could assist foldedness of the antigen. Shorter connections between DA4B and the N1 NA head domain (residues 83-469) were therefore more thoroughly characterized using flexible exogenous linkers of 6 (DA4B_L6_83-N1-MI15), 8 (DA4B_L8_83-N1-MI15) and 10 (DA4B_L10_83-N1-MI15) residues. After expression and purification, catalytic activity was measured to be identical for DA4B_L2_70-N1-MI15 and all three modified constructs (FIG. 4B). Binding to oseltamivir was measured, which showed improvements in binding intensity for NAs with shorter DA4B-antigen connections relative to DA4B_L2_70-N1-MI15 (Table 8). ITF-based thermal denaturations were next measured in the presence of calcium chloride, which showed similar melting temperatures but different baseline shifts in fluorescence (Table 9). Notably, fluorescence baselines were most blue-shifted for DA4B_L8_83-N1-MI15 and DA4B_L6_83-N1-MI15, which had the shortest connections, and most redshifted for DA4B_L2_70-N1-MI15, with DA4B_L10_83-N1-MI15 in between. Blue-shifted fluorescence indicates improved burial of tryptophan residues, suggesting conformational differences between many of these samples within the globular NA head, which is the only portion of these sequences that contains tryptophan residues, with variable solvent exposure of some tryptophan residues observed in literature¹⁰.

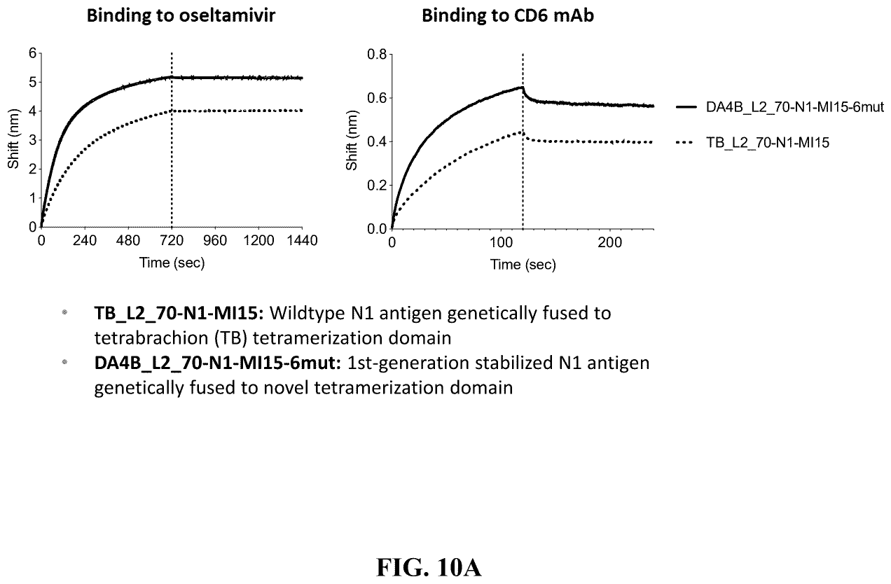

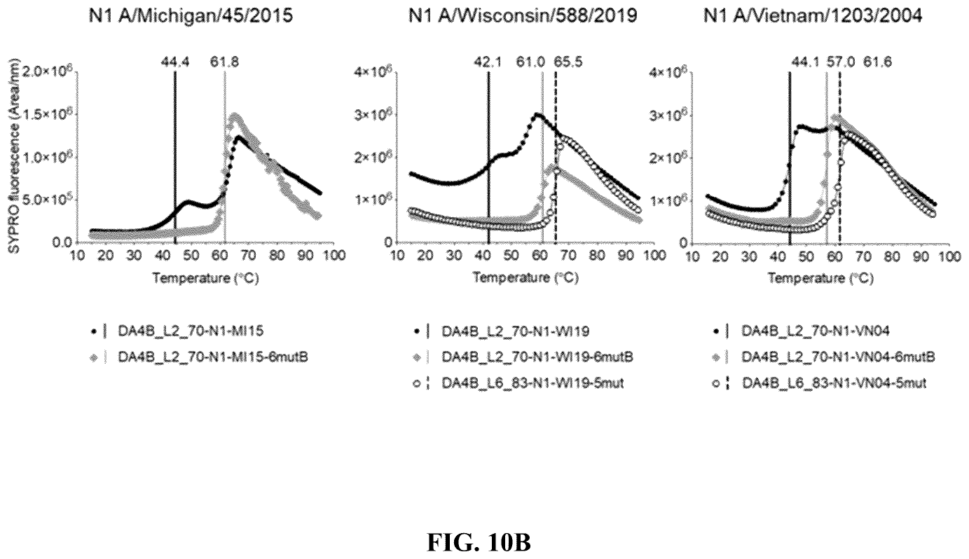

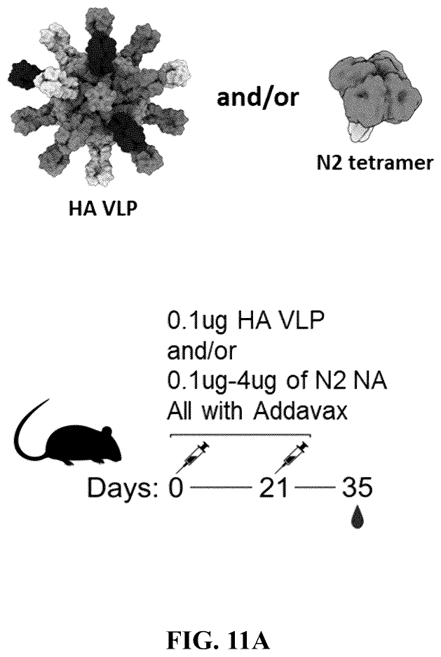

The NA head contains binding sites for Ca²⁺ ions, with exposure to ethylenediaminetetraacetic acid (EDTA) characterized to strip the antigen of Ca²⁺ ions and lead to decreased antigen stability and altered foldedness¹⁰. Binding to oseltamivir was measured in the presence of EDTA to inform antigenicity after stress from chelators, which showed generally decreased signal relative to previous measurements (Table 8). Nonetheless, constructs with shorter connections showed higher binding signals, with DA4B_L6_83-N1-MI15 showing the highest signal. ITF-based thermal denaturations were measured in the presence of 5 mM EDTA, which greatly decreased melting temperatures for all samples relative to experiments containing added calcium chloride, but showed an inverse relationship between length of DA4B-antigen connection and melting temperature (Table 9). Further, DA4B_L6_83-N1-MI15 showed more blue-shifted baseline fluorescence relative to other samples (Table 9). Finally, binding to the quaternary-specific monoclonal antibody CD6¹¹ was measured for DA4B_L2_70-N1-MI15 and DA4B_L6_83-N1-MI15 in the presence and absence of EDTA, showing improved binding in both conditions. As shown in Table 10 and FIG. 10A, binding to CD6 from DA4B-based designs that test different connections between DA4B and seasonal NA head domains. Data was collected in the presence of either calcium chloride or EDTA. These data show that DA4B soluble tetramers allow for N1 NA antigens with similar binding to CD6 compared to equivalent TB-based constructs. Further, these data show that modifications to the connection between N1 NA head domains and DA4B can improve binding to CD6, which indicate improved conformational closure of the NA head domains. As well, these data show that addition of stabilizing mutations to N1 NA head domains can also improve binding to CD6. In all, shorter DA4B-antigen connections were surprisingly seen to improve many biochemical properties of a representative N1 antigen, with a flexible 6-residue linker identified as particularly promising.

TABLE 10
Binding of select N1 constructs to CD6 mAb using BLI

| Sample | Shift (nm), CaCl2 | Shift (nm), EDTA |
|---|---|---|
| TB_L2_70-N1-MI15 | 0.42278102 | 0.19189564 |
| DA4B_L2_70-N1-MI15 | 0.41290827 | 0.14820199 |
| DA4B_L6_83-N1-MI15 | 0.60028115 | 0.31087476 |
| DA4B_L2_70-N1-MI15-6mutB | 0.6222951 | 0.24417043 |

The impact of different connections between DA4B and antigens was similarly studied for N2 and B antigens. The previously mentioned DA4B-N2-DA21 construct with an 8-residue linker and a fragment from residues 83-469 (DA4B_L8_83-N2-DA21) was compared to a construct with an equivalent construct with a 10-residue linker (DA4B_L10_83-N2-DA21) and a construct with a 2-residue linker and a fragment from residues 65-469 (DA4B_L2_65-N2-DA21). Catalytic activity analysis of purified protein showed decreased catalytic activity from DA4B_L2_65-N2-DA21, with similar elevated activity seen for DA4B_L8_83-N2-DA21 and DA4B_L10_83-N2-DA21 (FIG. 4C). DA4B_L8_83-N2-DA21 and DA4B_L10_83-N2-DA21 were further compared, showing similar profiles for binding to oseltamivir (Table 8) and ITF-based thermal denaturation (Table 9).

For both By- and By-based constructs, two different connections were tested to DA4B, both of which used an 8-residue linker and an antigen fragment from residues 76-466 but varied in amino acid identity (DA4B_L8_75-Bv-CO17 and DA4B_L8A_75-Bv-CO17 for Bv, DA4B_L8_75-Bv-PH13 and DA4B_L8A_75-Bv-PH13 for By). All biochemical measurements were similar for constructs from the same B lineage as seen for catalytic activity (FIGS. 4D and 4E), binding to oseltamivir (Table 8), and ITF-based thermal denaturation (Table 9).

In all, suitable connections between DA4B and diverse seasonal antigens were identified, with N1 antigens seen to have a strong sensitivity to properties of the connection, which is consistent with the studied lower stability of N1 antigens^{6,10}.

### Example 5: Optimization of Stabilizing Mutations with the N1 Globular Head Domain

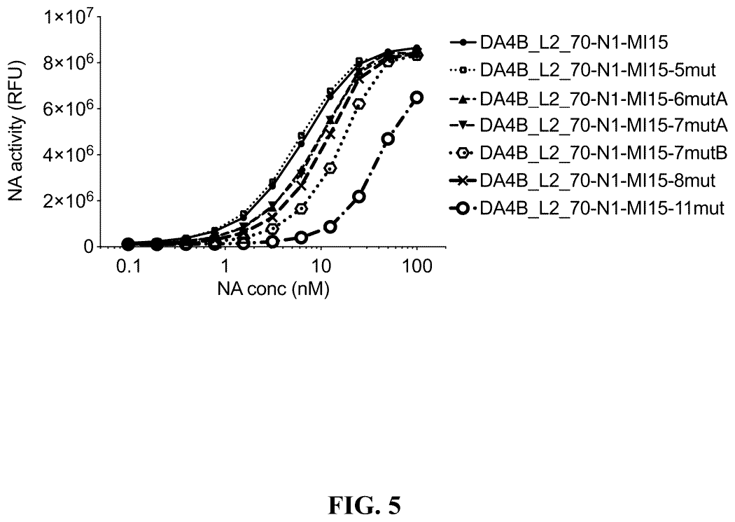

Multiple mutations were added in different combinations to DA4B_L2_70-N1-MI15, and purified protein for each design was analyzed in vitro. One design (DA4B_L2_70-N1-MI15-5mut) featured the addition of I99P, C161V, E165S, S172A and S196T and showed equivalent levels of catalytic activity to DA4B_L2_70-N1-MI15, however addition of T131Q on top of these five mutations (DA4B_L2_70-N1-MI15-6mutA) lowered catalytic activity moderately (FIG. 5). Further addition of V177I (DA4B_L2_70-N1-MI15-7mutA) or V177I and V205I (DA4B_L2_70-N1-MI15-8mut) to DA4B_L2_70-N1-MI15-6mutA did not impact catalytic activity, however addition of V453T to DA4B_L2_70-N1-MI15-6mutA (DA4B_L2_70-N1-MI15-7mutB) showed additional moderate decreases in catalytic activity. Addition of Y100L, Q408M and R419V to DA4B_L2_70-N1-MI15-8mut (DA4B_L2_70-N1-MI15-11mut) showed an additional decrease in catalytic activity. All constructs with added stabilizing mutations showed decreased binding signal to oseltamivir relative to DA4B_L2_70-N1-MI15. As shown in Table 11, binding to oseltamivir from select N1 antigens containing stabilizing mutations and genetically fused to DA4B. Data was collected in the presence of either calcium chloride or EDTA. These data show that unlike some N1 designs presented in Table 2, many combinations of stabilizing mutations are not effective at maintaining binding to oseltamivir. T13IQ was seen to be associated with red-shifted ITF signals, while the addition of V177I without V205I appeared to be associated with blue-shifted ITF signals relative to comparable samples. As shown in Table 12, ITF-based baseline measurements from select N1 antigens containing stabilizing mutations and genetically fused to DA4B. Data was measured in the presence of calcium chloride at 25° C. These data show that many designs show red-shifted fluorescence signals in contrast to other N1 designs shown in Table 9, suggested disordering of tryptophan residues. Melting temperatures measured using SYPRO fluorescence further revealed differences in stability. SYPRO-based melting temperatures from select N1 antigens containing stabilizing mutations and genetically fused to DA4B are shown in Table 13. Data was collected in the presence of either calcium chloride or EDTA, and samples that showed multiple melting transitions are noted with comma-separated values. These data show that all the constructs with added mutations improve thermal stability relative to DA4B_L2_70-N1-MI15. In the presence of added Ca²⁺ ions, all constructs with stabilizing mutations showed improved melting properties over DA4B_L2_70-N1-MI15 by removing an early melting transition. In the presence of 5 mM EDTA, all constructs showed dramatic increases in thermal stability, with major melting transitions shown to increase by at least 20° C., while DA4B_L2_70-N1-MI15-5mut maintained an early melting transition.

TABLE 11
Binding of select N1 constructs to oseltamivir using BLI

| Sample | Shift (nm), CaCl2 | Shift (nm), EDTA |
|---|---|---|
| DA4B_L2_70-N1-MI15 | 5.22905753 | 3.06831479 |
| DA4B_L2_70-N1-MI15-5mut | 4.61450209 | 0.30511269 |
| DA4B_L2_70-N1-MI15-7mutA | 4.75170021 | 1.41439668 |
| DA4B_L2_70-N1-MI15-8mut | 4.66921007 | 1.68977378 |
| DA4B_L2_70-N1-MI15-11mut | 4.573214 | 0.26754124 |

TABLE 12
ITF baseline measurements for select N1 constructs:

| Sample | Baseline near 25 C. (CaCl2) |
|---|---|
| DA4B_L2_70-N1-MI15 | 349.9 |
| DA4B_L2_70-N1-MI15-5mut | 351.4 |
| DA4B_L2_70-N1-MI15-6mutA | 351.5 |
| DA4B_L2_70-N1-MI15-7mutA | 350.3 |
| DA4B_L2_70-N1-MI15-7mutB | 352.0 |
| DA4B_L2_70-N1-MI15-8mut | 351.7 |
| DA4B_L2_70-N1-MI15-11mut | 351.0 |

TABLE 13
SYPRO thermal denaturation measurements
for select N1 constructs:

Melting temperatures

| Sample | CaCl2 | EDTA |
|---|---|---|
| DA4B_L2_70-N1-MI15 | 45.6, 64.9 | 40.1 |
| DA4B_L2_70-N1-MI15-5mut | 65.1 | 44.4, 64.5 |
| DA4B_L2_70-N1-MI15-6mutA | 68 | 65.6 |
| DA4B_L2_70-N1-MI15-7mutA | 66.4 | 64 |
| DA4B_L2_70-N1-MI15-7mutB | 67.7 | 66.2 |
| DA4B_L2_70-N1-MI15-8mut | 66.6 | 64.1 |
| DA4B_L2_70-N1-MI15-11mut | 64.8 | 61.7 |

TABLE 14
SYPRO thermal denaturation measurements
for select N1 constructs:

Melting temperatures

| Sample | CaCl2 | EDTA |
|---|---|---|
| DA4B_L2_70-N1-MI15 | 44.4, 63.4 | 38.7 |
| DA4B_L2_70-N1-MI15-6mutB | 61.8 | 60.1 |

From these data an additional construct was designed to further improve stability while maintaining high levels of catalytic activity. From seeing T131Q decrease catalytic activity and red-shift ITF signals, as well as V177J not impacting catalytic activity while blue-shifting ITF signals, V177J was added to DA4B_L2_70-N1-MI15-5mut to generate DA4B_L2_70-N1-MI15-6mutB. This construct showed equivalent levels of catalytic activity to DA4B_L2_70-N1-MI15 (FIG. 4B) and, similarly to DA4B_L6_83-N1-MI15, showed elevated binding to oseltamivir (Table 8) and CD6 (Table 10), and strongly blue-shifted ITF signals (Table 9). Further, melting temperatures measured through SYPRO fluorescence were similarly high in the presence of either Ca²⁺ or EDTA, showing a >20° C. improvement in melting temperature over DA4B_L2_70-N1-MI15 in the presence of EDTA. Table 14 and FIG. 10B show SYPRO-based melting temperatures from select N1 antigens containing stabilizing mutations and genetically fused to DA4B. Data was collected in the presence of either calcium chloride or EDTA. These data show that addition of 6mutB mutations (I99P, C161V, E165S, S172A, V177I and S196T) to DA4B_L2_70-N1-MI15 greatly improves thermal stability.

### Example 6: Immunogenicity of Soluble NA Tetramers

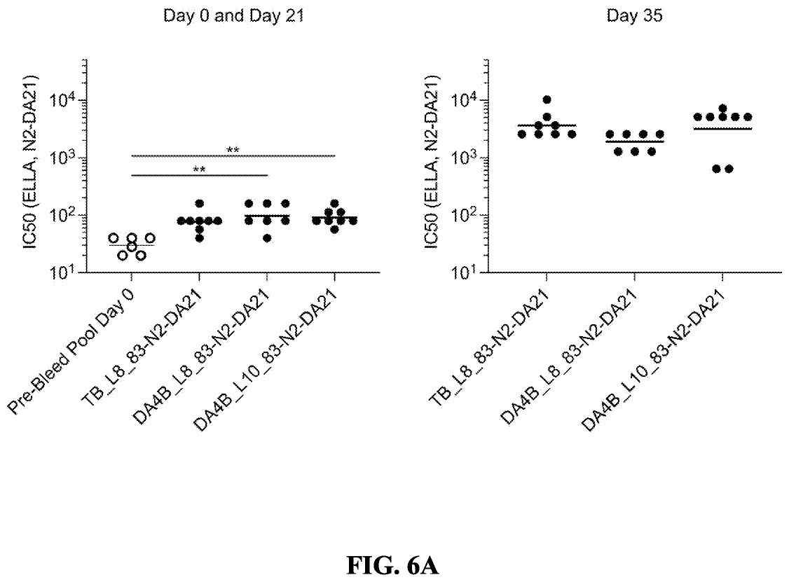

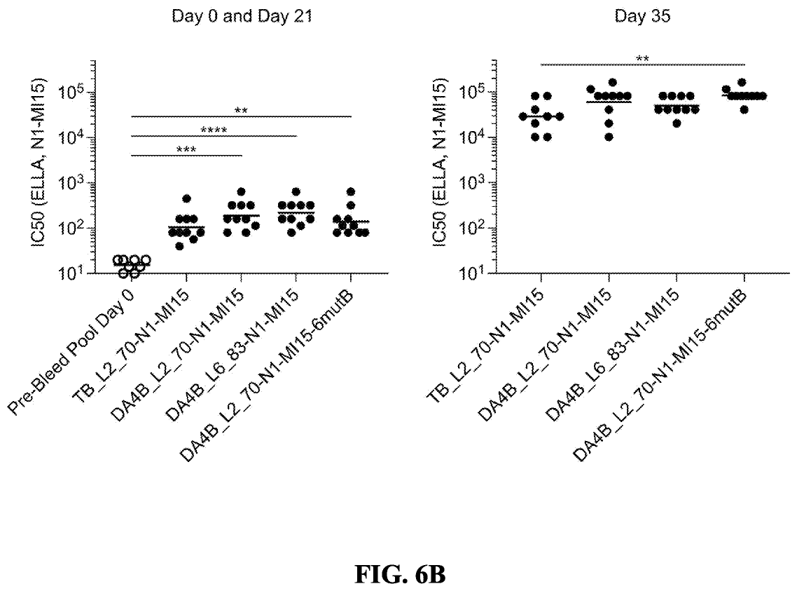

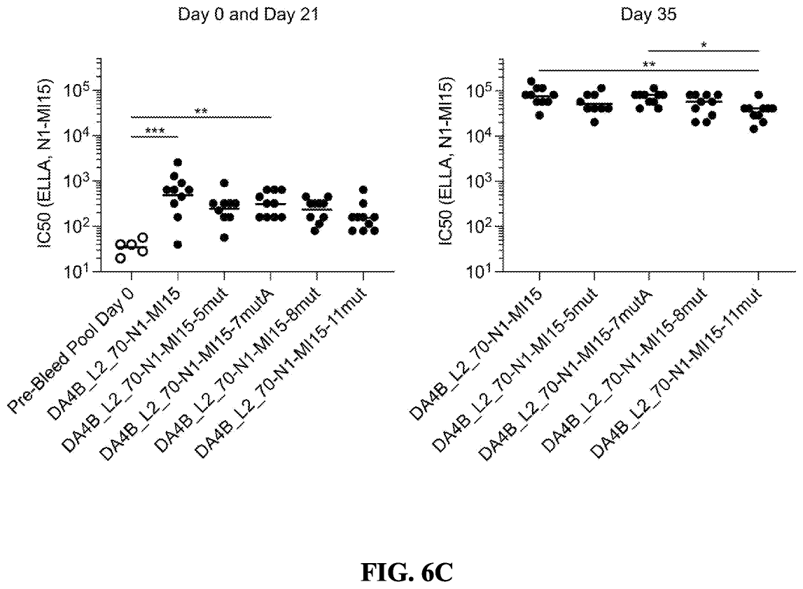

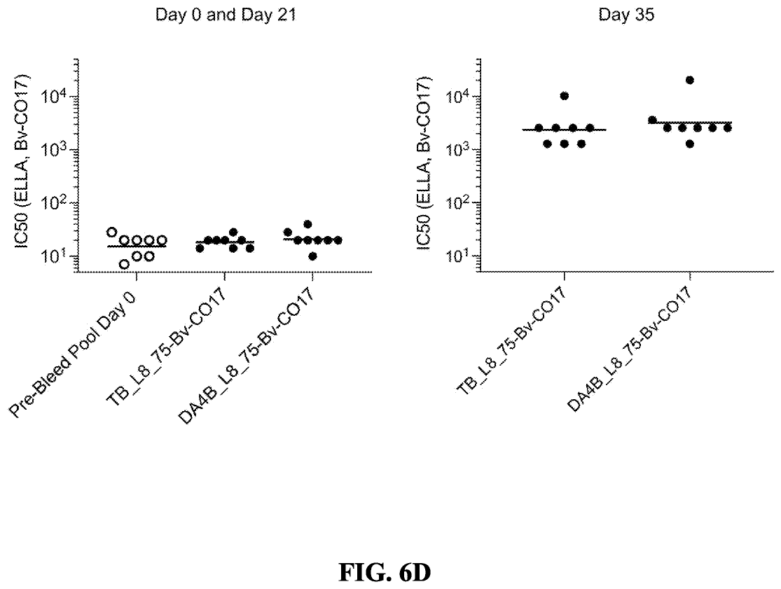

Immunogenicity was compared between different N2 constructs using TB_L8_83-N2-DA21, DA4B_L8_83-N2-DA21 and DA4B_L10_83-N2-DA21 in mice. Comparison between TB_L8_83-N2-DA21 and DA4B_L8_83-N2-DA21 was used to measure immunogenic differences between TB and DA4B, while comparison between DA4B_L8_83-N2-DA21 and DA4B_L10_83-N2-DA21 was used to measure whether changes to the linkers within the design could impact immunogenicity. Mice were immunized twice (day 0 and day 21) with 1 μg of each purified protein that was adjuvanted with Addavax™. ELLA titers were measured against live, native strain-matched N2-DA21 virus on serum samples from days 21 and 35, as well as from pooled serum samples from day 0 to approximate the assay baseline (FIG. 6A, Table 15). As shown in Table 15, immunogenicity statistics, which were performed using a Kruskal-Wallis test followed by Dunn's multiple comparison.

TABLE 15
Statistics from immunogenicity studies:

| | | | | |
|---|---|---|---|---|
| | | | | Adjusted |
| Sample comparison | Day | Significant | Summary | P Value |

N2-DA21 Study

| | | | | |
|---|---|---|---|---|
| TB_L8_83-N2-DA21 vs. | 21 | No | ns | >0.9999 |
| DA4B_L8_83-N2-DA21 | | | | |
| TB_L8_83-N2-DA21 vs. | 21 | No | ns | >0.9999 |
| DA4B_L10_83-N2-DA21 | | | | |
| TB_L8_83-N2-DA21 vs. Pre-Bleed | 21 | No | ns | 0.0502 |
| Pool Day 0 | | | | |
| DA4B_L8_83-N2-DA21 vs. | 21 | No | ns | >0.9999 |
| DA4B_L10_83-N2-DA21 | | | | |
| DA4B_L8_83-N2-DA21 vs. Pre- | 21 | Yes | ** | 0.0039 |
| Bleed Pool Day 0 | | | | |
| DA4B_L10_83-N2-DA21 vs. Pre- | 21 | Yes | ** | 0.0052 |
| Bleed Pool Day 0 | | | | |
| TB_L8_83-N2-DA21 vs. | 35 | No | ns | 0.177 |
| DA4B_L8_83-N2-DA21 | | | | |
| TB_L8_83-N2-DA21 vs. | 35 | No | ns | >0.9999 |
| DA4B_L10_83-N2-DA21 | | | | |
| DA4B_L8_83-N2-DA21 vs. | 35 | No | ns | 0.0795 |
| DA4B_L10_83-N2-DA21 | | | | |

N1-MI15 Study #1

| | | | | |
|---|---|---|---|---|
| Pre-Bleed Pool Day 0 vs. TB_L2_70- | 21 | No | ns | 0.0713 |
| N1-MI15 | | | | |
| Pre-Bleed Pool Day 0 vs. | 21 | Yes | *** | 0.0003 |
| DA4B_L2_70-N1-MI15 | | | | |
| Pre-Bleed Pool Day 0 vs. | 21 | Yes | **** | <0.0001 |
| DA4B_L6_83-N1-MI15 | | | | |
| Pre-Bleed Pool Day 0 vs. | 21 | Yes | ** | 0.0099 |
| DA4B_L2_70-N1-MI15-6mutB | | | | |
| TB_L2_70-N1-MI15 vs. | 21 | No | ns | >0.9999 |
| DA4B_L2_70-N1-MI15 | | | | |
| TB_L2_70-N1-MI15 vs. | 21 | No | ns | 0.507 |
| DA4B_L6_83-N1-MI15 | | | | |
| TB_L2_70-N1-MI15 vs. | 21 | No | ns | >0.9999 |
| DA4B_L2_70-N1-MI15-6mutB | | | | |
| DA4B_L2_70-N1-MI15 vs. | 21 | No | ns | >0.9999 |
| DA4B_L6_83-N1-MI15 | | | | |
| DA4B_L2_70-N1-MI15 vs. | 21 | No | ns | >0.9999 |
| DA4B_L2_70-N1-MI15-6mutB | | | | |
| DA4B_L6_83-N1-MI15 vs. | 21 | No | ns | >0.9999 |
| DA4B_L2_70-N1-MI15-6mutB | | | | |
| TB_L2_70-N1-MI15 vs. | 35 | No | ns | 0.1057 |
| DA4B_L2_70-N1-MI15 | | | | |
| TB_L2_70-N1-MI15 vs. | 35 | No | ns | >0.9999 |
| DA4B_L6_83-N1-MI15 | | | | |
| TB_L2_70-N1-MI15 vs. | 35 | Yes | ** | 0.0057 |
| DA4B_L2_70-N1-MI15-6mutB | | | | |
| DA4B_L2_70-N1-MI15 vs. | 35 | No | ns | >0.9999 |
| DA4B_L6_83-N1-MI15 | | | | |
| DA4B_L2_70-N1-MI15 vs. | 35 | No | ns | >0.9999 |
| DA4B_L2_70-N1-MI15-6mutB | | | | |
| DA4B_L6_83-N1-MI15 vs. | 35 | No | ns | 0.2842 |
| DA4B_L2_70-N1-MI15-6mutB | | | | |

N1-MI15 Study #2

| | | | | |
|---|---|---|---|---|
| Pre-Bleed Pool Day 0 vs. | 21 | Yes | *** | 0.0003 |
| DA4B_L2_70-N1-MI15 | | | | |
| Pre-Bleed Pool Day 0 vs. | 21 | No | ns | 0.0522 |
| DA4B_L2_70-N1-MI15-5mut | | | | |
| Pre-Bleed Pool Day 0 vs. | 21 | Yes | ** | 0.0079 |
| DA4B_L2_70-N1-MI15-7mutA | | | | |
| Pre-Bleed Pool Day 0 vs. | 21 | No | ns | 0.0612 |
| DA4B_L2_70-N1-MI15-8mut | | | | |
| Pre-Bleed Pool Day 0 vs. | 21 | No | ns | 0.7502 |
| DA4B_L2_70-N1-MI15-11mut | | | | |
| DA4B_L2_70-N1-MI15 vs. | 21 | No | ns | >0.9999 |
| DA4B_L2_70-N1-MI15-5mut | | | | |
| DA4B_L2_70-N1-MI15 vs. | 21 | No | ns | >0.9999 |
| DA4B_L2_70-N1-MI15-7mutA | | | | |
| DA4B_L2_70-N1-MI15 vs. | 21 | No | ns | >0.9999 |
| DA4B_L2_70-N1-MI15-8mut | | | | |
| DA4B_L2_70-N1-MI15 vs. | 21 | No | ns | 0.0677 |
| DA4B_L2_70-N1-MI15-11mut | | | | |
| DA4B_L2_70-N1-MI15-5mut vs. | 21 | No | ns | >0.9999 |
| DA4B_L2_70-N1-MI15-7mutA | | | | |
| DA4B_L2_70-N1-MI15-5mut vs. | 21 | No | ns | >0.9999 |
| DA4B_L2_70-N1-MI15-8mut | | | | |
| DA4B_L2_70-N1-MI15-5mut vs. | 21 | No | ns | >0.9999 |
| DA4B_L2_70-N1-MI15-11mut | | | | |
| DA4B_L2_70-N1-MI15-7mutA vs. | 21 | No | ns | >0.9999 |
| DA4B_L2_70-N1-MI15-8mut | | | | |
| DA4B_L2_70-N1-MI15-7mutA vs. | 21 | No | ns | 0.975 |
| DA4B_L2_70-N1-MI15-11mut | | | | |
| DA4B_L2_70-N1-MI15-8mut vs. | 21 | No | ns | >0.9999 |
| DA4B_L2_70-N1-MI15-11mut | | | | |
| DA4B_L2_70-N1-MI15 vs. | 35 | No | ns | >0.9999 |
| DA4B_L2_70-N1-MI15-5mut | | | | |
| DA4B_L2_70-N1-MI15 vs. | 35 | No | ns | >0.9999 |
| DA4B_L2_70-N1-MI15-7mutA | | | | |
| DA4B_L2_70-N1-MI15 vs. | 35 | No | ns | 0.8315 |
| DA4B_L2_70-N1-MI15-8mut | | | | |
| DA4B_L2_70-N1-MI15 vs. | 35 | Yes | ** | 0.0095 |
| DA4B_L2_70-N1-MI15-11mut | | | | |
| DA4B_L2_70-N1-MI15-5mut vs. | 35 | No | ns | >0.9999 |
| DA4B_L2_70-N1-MI15-7mutA | | | | |
| DA4B_L2_70-N1-MI15-5mut vs. | 35 | No | ns | >0.9999 |
| DA4B_L2_70-N1-MI15-8mut | | | | |
| DA4B_L2_70-N1-MI15-5mut vs. | 35 | No | ns | >0.9999 |
| DA4B_L2_70-N1-MI15-11mut | | | | |
| DA4B_L2_70-N1-MI15-7mutA vs. | 35 | No | ns | >0.9999 |
| DA4B_L2_70-N1-MI15-8mut | | | | |
| DA4B_L2_70-N1-MI15-7mutA vs. | 35 | Yes | * | 0.0359 |
| DA4B_L2_70-N1-MI15-11mut | | | | |
| DA4B_L2_70-N1-MI15-8mut vs. | 35 | No | ns | >0.9999 |
| DA4B_L2_70-N1-MI15-11mut | | | | |

Bv-CO17 Study

| | | | | |
|---|---|---|---|---|
| TB_L8_75-Bv-CO17 vs. Pre-Bleed | 0 and 21 | No | ns | >0.9999 |
| Pool Day 0 | | | | |
| TB_L8_75-Bv-CO17 vs. | 21 | No | ns | 0.7479 |
| DA4B_L8_75-Bv-CO17 | | | | |
| TB_L8_75-Bv-CO17 vs. | 35 | No | ns | >0.9999 |
| DA4B_L8_75-Bv-CO17 | | | | |

On day 21, ELLA titers were slightly elevated for all NA samples above the day 0 baseline, with both DA4B antigens showing significance from the day 0 pool and no significant differences seen between any of the NA groups. ELLA titers were thoroughly elevated for all N2 samples on day 35, with no significant differences seen between all NA groups. In all, similar immunogenicity was observed between N2 constructs using either TB or DA4B, and modification of the connections used in DA4B-based N2 constructs did not impact immunogenicity, which is reflected in similar in vitro measurements.

In one immunogenicity study for N1 constructs, the impact of tetramerization domain used and addition of stabilizing design features was explored using TB_L2_70-N1-MI15, DA4B_L2_70-N1-MI15, DA4B_L6_83-N1-MI15 and DA4B_L2_70-N1-MI15-6mutB. The same dosing and bleed schedule was used as described for the previous study, with ELLA titers measured against live, native strain-matched N1-MI15 virus (FIG. 6B, Table 15). On day 21, ELLA titers were elevated for all NA samples above the day 0 baseline, with all DA4B antigens showing significance from the day 0 pool and no significant differences seen between any of the NA groups. Similar to N2, ELLA titers were more strongly elevated on day 35, with DA4B_L2_70-N1-MI15-6mutB ELLA titers measured to be higher than TB_L2_70-N1-MI15 with statistical significance.

In a different immunogenicity study for N1 constructs, DA4B_L2_70-N1-MI15 was compared to alternative N1 designs with stabilizing mutations (DA4B_L2_70-N1-MI15-5mut, DA4B_L2_70-N1-MI15-7mutA, DA4B_L2_70-N1-MI15-8mut, DA4B_L2_70-N1-MI15-11mut), with such designs found to have lower levels of binding to oseltamivir, and in some cases lower catalytic activity. The same dosing and bleed schedule was used as described for the previous studies, with ELLA titers measured against live, native strain-matched N1-MI15 (FIG. 6C, Table 15). On day 21, all groups showed elevated ELLA titers over baseline but with differences in measured geometric mean titers (GMTs), with only DA4B_L2_70-N1-MI15 and DA4B_L2_70-N1-MI15-7mutA showing statistical differences from baseline, and DA4B_L2_70-N1-MI15-11mut seen to have the lowest GMT. ELLA titers were strongly boosted for all samples on day 35, but showed some significant differences in response levels. Surprisingly, DA4B_L2_70-N1-MI15-11mut was found to have lower GMTs than all other samples, and showed statistically significant differences from both DA4B_L2_70-N1-MI15 and DA4B_L2_70-N1-MI15-7mutA. This difference appears most likely explained by the lower catalytic activity observed from DA4B_L2_70-N1-MI15-11mut (FIG. 5).

The immunogenicity of similar By constructs using either TB or DA4B was also compared using TB_L8_75-Bv-CO17 and DA4B_L8_75-Bv-CO17, with animal work performed as described in experiments above and ELLA titers measured against live, native strain-matched By-CO17 virus. Titers for both groups were similarly low on day 21, but strongly boosted after the second shot as measured on day 35 (FIG. 6D). No statistical differences were seen between each group within each time point (Table 15).

### Example 7: Stabilized N1 Soluble Tetramers

Two different N1 strains were selected for analysis of mutations: A/Wisconsin/588/2019 (WI19) and A/Vietnam/1203/2004 (VN04). WI19 is a recent seasonal H1N1 strain that was previously recommended by WHO for inclusion in seasonal vaccines, while VN04 is a widely-studied avian H5N1 strain with potential for zoonotic spillover into humans. The head domains of WI19 and VN04 are 89.1% identical, in all representing divergent NA sequences from unique backgrounds. Both 6mutB (I99P, C161V, E165S, S172A, V177I and S196T) and 5mut (I99P, C161V, E165S, S172A and S196T) stabilizing mutations were tested in the context of designs that used the L6 linker and the NA fragment start point at residue 83 (or residue 63 for VN04 due to a naturally shorter stalk sequence prior to the NA head domain), to show that designs could maintain high catalytic activity while improving thermal stability and ability to bind oseltamivir. To bridge to previous designs, equivalents of DA4B_L2_70-N1-MI15 (DA4B_L2_70-N1-WI19 and DA4B_L2_50-N1-VN04) and DA4B_L2_70-N1-MI15-6mutB (DA4B_L2_70-N1-WI19-6mutB and DA4B_L2_50-N1-VN04-6mutB) designs were generated for both WI19 and VN04 NA sequences.

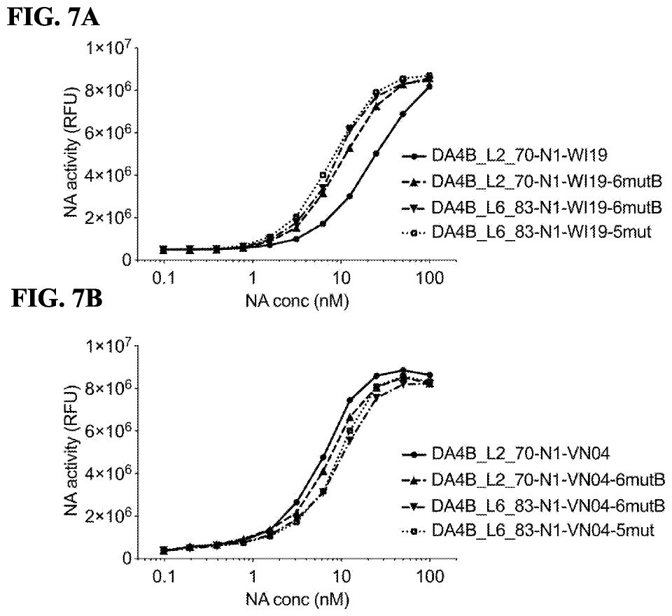

For WI19-based designs, a surprising increase in catalytic activity was observed for designs containing 6mutB or 5mut mutations relative to the DA4B_L2_70-N1-WI19 design (FIG. 7A), with greater than 2-fold higher catalytic activity observed for the stabilized designs at many dilutions. Further, levels of binding to oseltamivir were also increased for all stabilized designs relative to DA4B_L2_70-N1-WI19 (Table 16). Both the DA4B_L6_83-N1-WI19-6mutB and DA4B_L6_83-N1-WI19-5mut designs measured to have the highest level of binding. Melting temperatures were next assessed by denaturation in the presence of SYPRO dye, which showed dramatically increased stability for designs containing stabilizing mutations in the presence of either CaCl₂) or EDTA (FIG. 10B, Table 17). Surprisingly, both DA4B_L6_83-N1-WI19-6mutB and DA4B_L6_83-N1-WI19-5mut designs were measured to have higher melting temperatures than DA4B_L2_70-N1-WI19-6mutB, showing that the combination of stabilizing mutations and the shorter engineered connection can further optimize stability of soluble NA antigens.

For VN04-based designs, similar improvements in binding to oseltamivir (Table 16) and thermal stability (FIG. 10B, Table 17) were measured for designs containing stabilizing mutations relative to DA4B_L2_50-N1-VN04. Trends were also similar, with both DA4B_L6_63-N1-VN04-6mutB and DA4B_L6_63-N1-VN04-5mut showing improved binding to oseltamivir and higher melting temperatures relative to DA4B_L2_50-N1-VN04-6mutB. Catalytic activity was subtly decreased for designs containing stabilizing mutations relative to DA4B_L2_50-N1-VN04 but were less than a 2-fold decrease (FIG. 7B).

In all, these data both showed that these design features can be generalized to diverse N1 strains, and that the combination of stabilizing mutations and the engineered shorter connection between DA4B and the NA head assists antigenicity and stability relative to stabilizing mutations alone. Further, these data showed a surprising increase in catalytic activity for stabilized designs that were applied to the WI19 sequence. Combined with the lower melting temperatures measured for DA4B_L2_70-N1-WI19, these data suggest that stabilized designs have potential to more dramatically improve the foldedness of certain recombinant N1 NA antigens that inherently have lower thermal stability.

TABLE 16
Binding of select N1 constructs to oseltamivir using BLI

| Sample | Shift (nm), CaCl2 |
|---|---|
| WI19 strain | |
| DA4B_L2_70-N1-WI19 | 4.49247488 |
| DA4B_L2_70-N1-WI19-6mutB | 4.93666636 |
| DA4B_L6_83-N1-WI19-6mutB | 5.33776859 |
| DA4B_L6_83-N1-WI19-5mut | 5.19185874 |
| VN04 strain | |
| DA4B_L2_70-N1-VN04 | 4.03510567 |
| DA4B_L2_70-N1-VN04-6mutB | 5.22654806 |
| DA4B_L6_83-N1-VN04-6mutB | 5.3087156 |
| DA4B_L6_83-N1-VN04-5mut | 5.35803906 |

TABLE 17
SYPRO thermal denaturation measurements
for select N1 constructs

| | Melting | Melting |
|---|---|---|
| | temperatures | temperatures |
| Sample | (CaCl2) | (EDTA) |
| WI19 strain | | |
| DA4B_L2_70-N1-WI19 | 42.1, 55.6 | 30.9 |
| DA4B_L2_70-N1-WI19-6mutB | 61 | 57.7 |
| DA4B_L6_83-N1-WI19-6mutB | 64.4 | 62.1 |
| DA4B_L6_83-N1-WI19-5mut | 65.5 | 62.4 |
| VN04 strain | | |
| DA4B_L2_70-N1-VN04 | 44.1 | 41.2 |
| DA4B_L2_70-N1-VN04-6mutB | 57 | 49.2 |
| DA4B_L6_83-N1-VN04-6mutB | 60.8 | 56.7 |
| DA4B_L6_83-N1-VN04-5mut | 61.6 | 57.2 |

### Example 8: Modifications to DA4B Tetramerization Domain

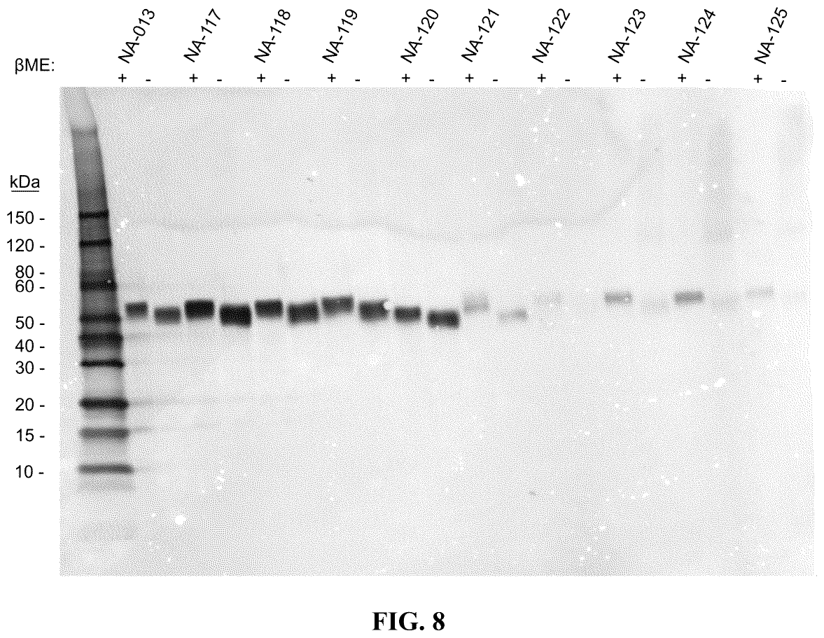

Removing residues from DA4B has potential to improve the ratio of antibodies focused on the NA head domain relative to DA4B. Modified versions of DA4B_L2_70-N1-MI15 were designed that removed progressive hydrophobic layers from the N-terminal end of the DA4B tetramerization domain, and the constructs were assessed by small-scale secretion from HEK293 cells (FIG. 8). All constructs with less residues removed maintained high levels of binding to oseltamivir from cell supernatants (Table 18). The shortest construct tested that provided quality yield and antigenicity removed 15 residues (DA4B-del15_L2_70-N1-MI15), suggesting that these 15 residues may not be necessary for proper tetramerization of DA4B and/or its support of NA antigens through genetic fusion.

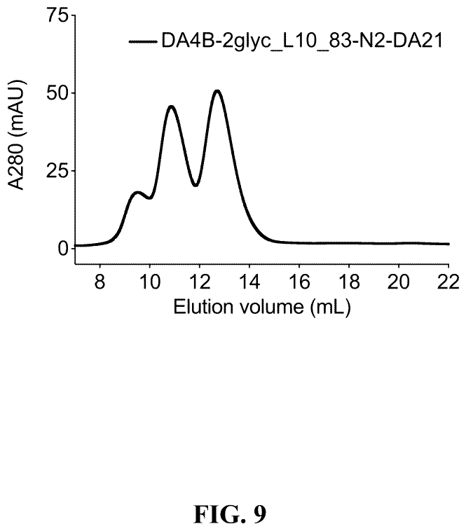

While DA4B naturally contains two N-linked “NxT” glycosylation sites, further addition of glycosylation sites could have potential to similarly improve the ratio of antibodies focused on the NA head domain. Based on structural analysis, a version of DA4B was designed that contains two additional N-linked glycosylation sites. While these modified DA4Bs could support expression of soluble antigen fusions for N1, N2 and B NAs, genetic fusion to N2 antigens led to proteins with poor SEC profiles (FIG. 9) that were not completely consistent with homogenous well-formed tetramers.

TABLE 18
Binding of N1 constructs with shortened DA4B versions
as supernatants to oseltamivir using BLI

| Sample | Shift (nm) |
|---|---|
| DA4B_L2_70-N1-MI15 | 4.27186656 |
| DA4B-del3_L2_70-N1-MI15 | 4.35545992 |
| DA4B-del7_L2_70-N1-MI15 | 4.30912768 |
| DA4B-del11_L2_70-N1-MI15 | 4.36893494 |
| DA4B-del15_L2_70-N1-MI15 | 4.36900024 |

### Example 9: Combination Vaccines

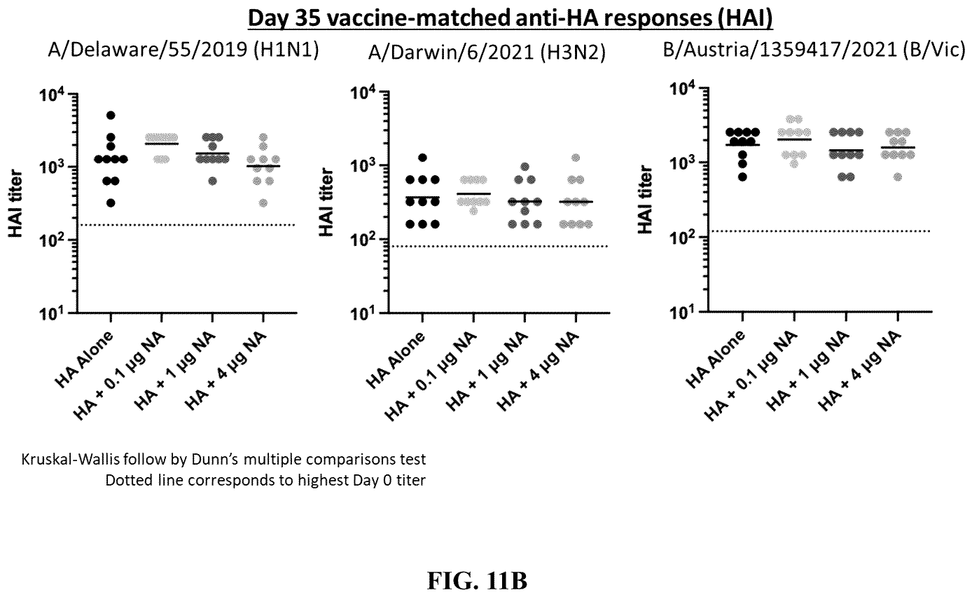

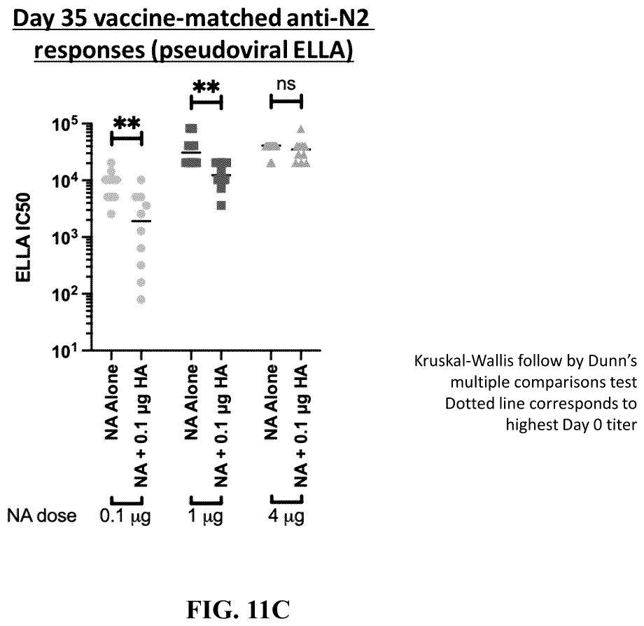

To test whether DA4B-based soluble influenza NA tetramers can maintain immunogenicity in the presence of influenza HA-based vaccines, the immunogenicity of formulations containing an N2 soluble tetramer (DA4B_L8_83-N2-DA21) and/or a two-component virus-like particle (VLP) nanostructure (derived from I53-50 [Bale et al.]) displaying four seasonal HA antigens were tested. This “HA VLP” nanostructure comprises H1, H3, By and By HA antigens and co-assembles all four HAs in equimolar ratios as a “mosaic” VLP formulation (Ref 15). Soluble NA tetramer (DA4B_L8_83-N2-DA21) was dosed at either 0.1 μg, 1 μg or 4 μg of total protein. Each dosage of NA tetramer (DA4B_L8_83-N2-DA21) was analyzed either with or without 0.1 μg of HA VLP nanostructure, and a group with just 0.1 μg of the HA VLP nanostructure was included as a control. Groups of mice were immunized twice with each formulation on days 0 and 21, and all groups were adjuvanted with Addavax™. Mice were bled on day 35 and serum was assessed for qualitative responses against both HA and NA antigens (FIG. 11A). Hemagglutination inhibition (HAI) was measured comparing live influenza viruses with equivalent H1, H3 and By HAs to antigens included in the HA VLP nanostructure. Addition of NA at various dosages showed no impact on HAI titers against each virus tested (FIG. 11B). ELLA titers were tested against pseudoviruses displaying N2 NAs matched to DA4B_L8_83-N2-DA21 (A/Darwin/6/2021). The ELLA titers were detected in all groups, with titers from subject groups administered 0.1 μg of NA tetramer (DA4B_L8_83-N2-DA21) lower than from subject groups administered higher dosages of NA. When 0.1 μg or 1 μg of NA tetramer (DA4B_L8_83-N2-DA21) was administered, decreased ELLA titers were detected when NA tetramer (DA4B_L8_83-N2-DA21) was co-formulated with HA VLP relative to NA alone. However, when NA tetramer (DA4B_L8_83-N2-DA21) was administered at 4 μg, similar ELLA titers were seen with or without co-formulation with the HA VLP nanostructure (FIG. 11C). In all, these data show that it is possible to simultaneously elicit potent responses against HA and NA from a single vaccine co-formulated with DA4B-based soluble NA tetramers with HA-based vaccines.

### REFERENCES

1. Stetefeld et al, Crystal structure of a naturally occurring parallel right-handed coiled coil tetramer, Nat. Struct. Biol., 7(9):772-776 (2000).
2. Schmidt et al, A generic system for the expression and purification of soluble and stable influenza neuraminidase, PLoS One, 6(2):e16284 (2011).
3. Schotsaert et al, Long-Lasting Cross-Protection Against Influenza A by Neuraminidase and M2e-based immunization strategies, Sci. Rep., 6:24402 (2016).
4. Gao et al, Design of the Recombinant Influenza Neuraminidase Antigen Is Crucial for Its Biochemical Properties and Protective Efficacy, J. Virol., 95(24):e0116021 (2021).
5. Stadlbauer et al, Broadly protective human antibodies that target the active site of influenza virus neuraminidase, Science, 366(6464):499-504 (2019).
6. Sultana et al, Stability of neuraminidase in inactivated influenza vaccines, Vaccine, 32(19):2225-30 (2014).
7. Streicher et al, A phospha-oseltamivir-biotin conjugate as a strong and selective adhesive for the influenza virus, Bioorg. Med. Chem. Lett., 24(7):1805-7 (2014).
8. Xu et al, Structural characterization of the 1918 influenza virus H1N1 neuraminidase, J. Virol., 82(21):10493-501 (2008).
9. Gao et al, Design of the Recombinant Influenza Neuraminidase Antigen Is Crucial for Its Biochemical Properties and Protective Efficacy, J. Virol., 95(24):e0116021 (2021).
10. Ellis et al, Structure-based design of stabilized recombinant influenza neuraminidase tetramers, Nat. Commun., 13(1):1825 (2022).
11. Wan et al, Structural characterization of a protective epitope spanning A (H1N1) pdm09 influenza virus neuraminidase monomers, Nat. Commun., 6:6114 (2015).
12. Wohlbold et al, Vaccination with adjuvanted recombinant neuraminidase induces broad heterologous, but not heterosubtypic, cross-protection against influenza virus infection in mice, mBio, 6(2):e02556 (2015).
13. Mirdita et al, ColabFold: making protein folding accessible to all, Nat. Methods, 19(6):679-682 (2022).
14. Bale et al, Accurate design of megadalton-scale two-component icosahedral protein complexes, Science, 353(6297):389-394 (2016).
15. Boyoglu-Barum et al, Elicitation of broadly protective immunity to influenza by multivalent hemagglutinin nanoparticle vaccines. Nature, 592(7855):623-628 (2021).
16. Bale et al, Accurate design of megadalton-scale two-component icosahedral protein complexes. Science, 353(6297):389-394(2016).

### ENUMERATED EMBODIMENTS

The disclosure further provides the following enumerated embodiments:

Embodiment 1. A recombinant influenza neuraminidase (NA) tetramer, comprising four copies of a fusion protein, said fusion protein comprising, in N- to C-terminal order, a DA4B tetramerization domain and an influenza NA head domain, wherein the DA4B tetramerization domain comprises, consists essentially of, or consists of a DA4B polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5.

Embodiment 2. The tetramer of Embodiment 1, further comprising a linker between said DA4B tetramerization domain and said influenza NA head domain.

Embodiment 3. The tetramer of Embodiment 1 or Embodiment 2, wherein the DA4B tetramerization domain comprises between 30 and 60 amino residues.

Embodiment 4. The tetramer of any one of the previous Embodiment, wherein the DA4B polypeptide sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5.

Embodiment 5. The tetramer of any one of the previous Embodiments Embodiment, wherein the DA4B polypeptide sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1.

Embodiment 6. The tetramer of any one of the previous Embodiments, wherein the DA4B polypeptide sequence is SEQ ID NO: 5.

Embodiment 7. The tetramer of any one of the previous Embodiments, wherein the DA4B polypeptide sequence is SEQ ID NO: 1.

Embodiment 8. The tetramer of any one of the previous Embodiments, wherein the linker is polypeptide bond.

Embodiment 9. The tetramer of any one of the previous Embodiments, wherein the linker is SG.

Embodiment 10. The tetramer of any one of the previous Embodiments, wherein the linker is GGSGGSGG (SEQ ID NO: 11) or comprises the polypeptide sequence GGSGGSGG (SEQ ID NO: 11).

Embodiment 11. The tetramer of any one of the previous Embodiments, wherein the linker is ATPRGG (SEQ ID NO: 12) or comprises the polypeptide sequence ATPRGG (SEQ ID NO: 12).

Embodiment 12. The tetramer of any one of the previous Embodiments, wherein the linker is ATPRGSGG (SEQ ID NO: 13) or comprises the polypeptide sequence ATPRGSGG (SEQ ID NO: 13).

Embodiment 13. The tetramer of any one of the previous Embodiments, wherein the linker is GGATPRGSGG (SEQ ID NO: 14) or comprises the polypeptide sequence GGATPRGSGG (SEQ ID NO: 14).

Embodiment 14. The tetramer of any one of the previous Embodiments, wherein the influenza NA head domain comprises an NA polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15-31.

Embodiment 15. The tetramer of any one of the previous Embodiments, wherein the influenza NA head domain is a head domain of a subtype N1 influenza.

Embodiment 16. The tetramer of any one of the previous Embodiments, wherein the influenza NA head domain comprises three or more amino acid substitutions selected from the group consisting of I99P, Y100L, T131Q, C161V, E165S, S172A, V177I, S196T, V205I, Q408M, R419V, and V453T, numbered relative SEQ ID NO: 15.

Embodiment 17. The tetramer of any one of the previous Embodiments, wherein the influenza NA head domain comprises the amino acid substitutions, numbered relative SEQ ID NO: 15, selected from a) I99P, C161V, E165S, S172A, S196T; b) I99P, T131Q, C161V, E165S, S172A, S196T; c) I99P, T131Q, C161V, E165S, S172A, V177I, S196T; d) I99P, T131Q, C161V, E165S, S172A, S196T, V453T; e) I99P, T131Q, C161V, E165S, S172A, V177I, S196T, V205I; f) I99P, Y100L, T131Q, C161V, E165S, S172A, V177I, S196T, V205I, Q408M, R419V; g) I99P, C161V, E165S, S172A, V177I, S196T; h) I99P, C161V, S172A, S196T; or i) I99P, C161V, S172A, V177I, S196T.

Embodiment 18. The tetramer of any one of the previous Embodiments, wherein a) the NA head domains are in a closed conformation; b) the tetramer has increased thermal stability in the presence of chelating agent compared to a tetramer with tetrabrachion tetramerization domain; c) the tetramer has increased interaction to neuraminidase inhibitor compared to a tetramer with tetrabrachion tetramerization domain; and/or d) the tetramer is expressed in recombinant cell culture at increased yield compared to a tetramer with a tetrabrachion tetramerization domain.

Embodiment 19. A fusion protein comprised by the tetramer of any one of the previous Embodiments.

Embodiment 20. The fusion protein of Embodiment 19, wherein the fusion protein comprises, consisting essentially of, or consisting of a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 32-422 and 424-447.

Embodiment 21. A polynucleotide encoding the fusion protein of Embodiment 19 or Embodiment 20, optionally wherein said polynucleotide is an mRNA.

Embodiment 22. A delivery vehicle comprising the polynucleotide of Embodiment 21, optionally wherein said delivery vehicle is a lipid nanoparticle.

Embodiment 23. A pharmaceutical composition, comprising the tetramer of any one of the previous Embodiments, the fusion protein of any one of the previous Embodiments, the polynucleotide of Embodiment 21, or the delivery vehicles of Embodiment 22 composition of any of preceding Embodiment and a pharmaceutically acceptable carrier.

Embodiment 24. A method of immunizing a subject in need thereof against infection by influenza, the method comprising administering to the subject an effective amount of a composition of any one of the previous Embodiments.

Embodiment 25. A method of treating and/or preventing influenza in a subject in need thereof, the method comprising administering to the subject an effective amount of a composition of any one of the previous Embodiments.

Embodiment 26. The method of Embodiment 24 or Embodiment 25, wherein the method generates an antibody response to the tetramer equivalent to or greater than the antibody response to administration of, or at least as effectively as administering a tetramer with a tetrabrachion tetramerization domain.

Embodiment 27. A kit comprising a composition of any one of the previous Embodiments and instructions for use.

Embodiment 28. A host cell comprising the polynucleotide of Embodiment 21.

Embodiment 29. A method of producing a recombinant influenza neuraminidase (NA) tetramer, comprising culturing the host cell of Embodiment 28 in cell culture media, and harvesting the recombinant influenza neuraminidase (NA) tetramer from the cell culture media or the host cell.

Embodiment 30. The method of Embodiment 29, wherein the method produces an increased yield of tetramer compared to production of a tetramer with a tetrabrachion tetramerization domain.

Embodiment 31. A fusion protein, comprising a DA4B tetramerization domain and a heterologous polypeptide, wherein the DA4B tetramerization domain comprises, consists essentially of, or consists of a DA4B polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5.

Embodiment 32. A recombinant influenza neuraminidase (NA) polypeptide, comprising an influenza NA head domain comprising one or more amino acid substitutions selected from the group consisting of I99P, Y100L, T131Q, C161V, E165S, S172A, V177I, S196T, V205I, Q408M, R419V, and V453T, numbered relative SEQ ID NO: 15.

Embodiment 33. The polypeptide of Embodiment 32, wherein the influenza NA head domain comprises three or more amino acid substitutions selected from the group consisting of I99P, Y100L, T131Q, C161V, E165S, S172A, V177I, S196T, V205I, Q408M, R419V, and V453T, numbered relative SEQ ID NO: 15.

Embodiment 34. The polypeptide of Embodiment 32 or Embodiment 33, wherein the influenza NA head domain comprises the amino acid substitutions, numbered relative SEQ ID NO: 15, selected from a) I99P, C161V, E165S, S172A, S196T; b) I99P, T131Q, C161V, E165S, S172A, S196T; c) I99P, T131Q, C161V, E165S, S172A, V177I, S196T; d) I99P, T131Q, C161V, E165S, S172A, S196T, V453T; e) I99P, T131Q, C161V, E165S, S172A, V177I, S196T, V205I; f) I99P, Y100L, T131Q, C161V, E165S, S172A, V177I, S196T, V205I, Q408M, R419V; g) I99P, C161V, E165S, S172A, V177I, S196T; h) I99P, C161V, S172A, S196T; or i) I99P, C161V, S172A, V177I, S196T.

Embodiment 35. The polypeptide of any one of Embodiments 32 to 34, wherein the influenza NA head domain comprises an NA polypeptide sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15-31, 423.

Embodiment 36. The polypeptide of any one of Embodiments 32 to 35, wherein the influenza NA head domain is a head domain of a subtype N1 influenza.

Embodiment 37. The polypeptide of any one of Embodiments 32 to 36, wherein a) the NA head domains are in a closed conformation; b) the tetramer has increased thermal stability in the presence of chelating agent compared to a tetramer with tetrabrachion tetramerization domain; c) the tetramer has increased interaction to neuraminidase inhibitor compared to a tetramer with tetrabrachion tetramerization domain; and/or d) the tetramer is expressed in recombinant cell culture at increased yield compared to a tetramer with a tetrabrachion tetramerization domain.

Embodiment 38. A fusion protein, comprising, in N- to C-terminal order, a tetramerization domain and an influenza NA polypeptide according to any one of Embodiments 32 to 37.

Embodiment 39. A recombinant influenza neuraminidase (NA) tetramer, comprising four copies of a fusion protein comprising, in N- to C-terminal order, a tetramerization domain and an influenza NA polypeptide according to any one of Embodiments 32 to 37.

Embodiment 40. A polynucleotide encoding the fusion protein of Embodiment 38, optionally wherein said polynucleotide is an mRNA.

Embodiment 41. A delivery vehicle comprising the polynucleotide of Embodiment 40, optionally wherein said delivery vehicle is a lipid nanoparticle.

Embodiment 42. A pharmaceutical composition, comprising the polypeptide of any one of Embodiments 32 to 37, the fusion protein of Embodiment 38, the recombinant influenza neuraminidase (NA) tetramer of Embodiment 39, the polynucleotide of Embodiment 40, or the delivery vehicle of Embodiment 41 and a pharmaceutically acceptable carrier.

Embodiment 43. A method of immunizing a subject in need thereof against infection by influenza, the method comprising administering to the subject an effective amount of the polypeptide of any one of Embodiments 32 to 37, the fusion protein of Embodiment 38, the recombinant influenza neuraminidase (NA) tetramer of Embodiment 39, the polynucleotide of Embodiment 40, or the delivery vehicle of Embodiment 41.

Embodiment 44. A method of treating and/or preventing influenza in a subject in need thereof, the method comprising administering to the subject an effective amount of the polypeptide of any one of Embodiments 32 to 37, the fusion protein of Embodiment 38, the recombinant influenza neuraminidase (NA) tetramer of Embodiment 39, the polynucleotide of Embodiment 40, or the delivery vehicle of Embodiment 41.

Embodiment 45. The method of Embodiment 43 or Embodiment 43, wherein the method generates an antibody response to the tetramer equivalent to or greater than the antibody response to administration of a tetramer with a tetrabrachion tetramerization domain.

Embodiment 46. A kit comprising the polypeptide of any one of Embodiments 32 to 37, the fusion protein of Embodiment 38, the recombinant influenza neuraminidase (NA) tetramer of

Embodiment 39, the polynucleotide of Embodiment 40, or the delivery vehicle of Embodiment 41 and instructions for use.

Embodiment 47. A host cell comprising the polynucleotide of Embodiment 40.

Embodiment 48. A method of producing a recombinant influenza neuraminidase (NA) tetramer, comprising culturing the host cell of Embodiment 47 in cell culture media, and harvesting the recombinant influenza neuraminidase (NA) tetramer from the cell culture media or the host cell.

Embodiment 49. The method of Embodiment 48, wherein the method produces an increased yield of tetramer compared to production of a tetramer with a tetrabrachion tetramerization domain.

Embodiment 50. A polypeptide, comprising the polypeptide sequence ATPRGG (SEQ ID NO: 12), ATPRGSGG (SEQ ID NO: 13), or GGATPRGSGG (SEQ ID NO: 14).

Embodiment 51. A recombinant influenza neuraminidase (NA) tetramer, comprising four copies of a fusion protein, said fusion protein comprising, in N- to C-terminal order, a tetramerization domain, a linker, and an influenza NA head domain, wherein the linker is or comprises the polypeptide sequence ATPRGG (SEQ ID NO: 12), ATPRGSGG (SEQ ID NO: 13), or GGATPRGSGG (SEQ ID NO: 14).

Embodiment 52. A pharmaceutical composition, comprising an recombinant influenza neuraminidase (NA) tetramer according to any one of Embodiments 1 to 31, Embodiment 39,

Embodiment 51, the polypeptide according to any one of Embodiments 32 to 37, or the fusion protein of Embodiment 38, and an influenza HA vaccine antigen.

Embodiment 53. The composition of Embodiment 52, wherein the influenza HA vaccine antigen is a protein nanostructure.

Embodiment 54. The composition of Embodiment 52 or Embodiment 53, wherein the protein nanostructure comprises a trimeric component comprising a trimer protein comprising a fusion protein, wherein the fusion protein comprises, in N- to C-terminal order, an influenza HA ectodomain, a polypeptide linker, and an assembly domain.

Embodiment 55. The composition of any one of Embodiments 52 to 54, wherein the protein nanostructure further comprises a pentameric component comprising a pentamer protein.

Embodiment 56. The composition of any one of Embodiments 54 to 55, wherein the assembly domain comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 458-460.

Embodiment 57. The composition of any one of Embodiments 54 to 56, wherein the influenza HA ectodomain comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 452-455, or 465.

Embodiment 58. The composition of any one of Embodiments 54 to 57, wherein the pentamer protein comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 461-463.

Embodiment 59. The composition of any one of Embodiments 54 to 58, wherein the trimer protein comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 448-451, or 464.

Embodiment 60. The composition of any one of Embodiments 54 to 59, wherein the polypeptide linker comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 456.

Embodiment 61. The composition of any one of Embodiments 54 to 60, wherein the polypeptide linker comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 457.

Embodiment 62. The composition of any one of Embodiments 54 to 61, wherein the composition comprises one or more pharmaceutically acceptable diluents, adjuvants, or excipients.

Embodiment 63. The composition of Embodiment 62, wherein the vaccine comprises one or more adjuvants.

Embodiment 64. The composition of Embodiment 63, wherein the one or more adjuvants is squalene, alum, SLA, GLA, R848, IMQ, 3M-052, CpG, saponin (QS21), or combinations thereof.

Embodiment 65. The composition of Embodiment 64, wherein the adjuvant is alum.

Embodiment 66. The composition of Embodiment 64, wherein the adjuvant is a squalene-based emulsion, optionally MF59®.

Embodiment 67. The composition of Embodiment 64, wherein the adjuvant is a squalene-based emulsion and a TLR4 agonist.

Embodiment 68. A method of immunizing a subject in need thereof against infection by influenza, the method comprising administering to the subject an effective amount of the composition according to any of one of Embodiments 52-67.

Embodiment 69. A method of treating and/or preventing influenza in a subject in need thereof, the method comprising administering to the subject an effective amount of the composition according to any of one of Embodiments 52-67.

Embodiment 70. The method of Embodiment 68 or 69, wherein the method generates an antibody response to the tetramer that is equivalent to or greater than the antibody response to administration of a tetramer with a tetrabrachion tetramerization domain.

Embodiment 71. The method of any one of Embodiments 68 to 70, wherein the method generates an antibody response to both influenza NA and influenza HA.

Embodiment 72. A protein nanostructure comprising a trimeric component comprising a trimer polypeptide and a pentameric component comprising a pentamer polypeptide, wherein the trimer polypeptide is a fusion protein comprising, in N- to C-terminal order, an influenza HA ectodomain, a polypeptide linker, and an assembly domain.

Embodiment 73. The nanostructure of Embodiment 72, wherein the assembly domain comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 458-460.

Embodiment 74. The nanostructure of any one of Embodiments 72 to 73, wherein the influenza HA ectodomain comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 452-455, or 465.

Embodiment 75. The nanostructure of any one of Embodiments 72 to 74, wherein the pentamer protein comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 461-463.

Embodiment 76. The nanostructure of any one of Embodiments 72 to 75, wherein the trimer protein comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 448-451, or 464.

Embodiment 77. The nanostructure of any one of Embodiments 72 to 76, wherein the polypeptide linker comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 456.

Embodiment 78. The nanostructure of any one of Embodiments 72 to 77, wherein the polypeptide linker comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 457.

Embodiment 79. A composition, method, or use as described herein.

While the invention has been described in connection with proposed specific embodiments thereof, it will be understood that it is capable of further modifications, and this application is intended to cover any variations, uses, or adaptations of the invention following, in general, the principles described herein, with such departures that may come within known or customary practice within the art to which the invention pertains.

Any tetramer, fusion protein, polynucleotide, delivery vehicle, VLP, composition or method disclosed herein is applicable to any herein-disclosed tetramer, fusion protein, polynucleotide, delivery vehicle, composition or method. In other words, any aspect or embodiment described herein can be combined with any other aspect or embodiment as disclosed herein.

1. A recombinant influenza neuraminidase (NA) tetramer, comprising four copies of a fusion protein, said fusion protein comprising, in N- to C-terminal order, a DA4B tetramerization domain and an influenza NA head domain,
wherein the DA4B tetramerization domain comprises, consists essentially of, or consists of a DA4B polypeptide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5.

2.-9. (canceled)

10. The tetramer of claim 1, further comprising a linker between said DA4B tetramerization domain and said influenza NA head domain, wherein the linker consists of GGSGGSGG (SEQ ID NO: 11) or comprises the polypeptide sequence GGSGGSGG (SEQ ID NO: 11), the linker consists of ATPRGG (SEQ ID NO: 12) or comprises the polypeptide sequence ATPRGG (SEQ ID NO: 12), the linker consists of ATPRGSGG (SEQ ID NO: 13) or comprises the polypeptide sequence ATPRGSGG (SEQ ID NO: 13), or the linker consists of GGATPRGSGG (SEQ ID NO: 14) or comprises the polypeptide sequence GGATPRGSGG (SEQ ID NO: 14).

11.-13. (canceled)

14. The tetramer of claim 1, wherein the influenza NA head domain comprises an NA polypeptide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 15-31 or 423.

15. (canceled)

16. The tetramer of claim 1, wherein the influenza NA head domain comprises three or more amino acid substitutions selected from the group consisting of I99P, Y100L, T131Q, C161V, E165S, S172A, V177I, S196T, V205I, Q408M, R419V, and V453T, numbered relative SEQ ID NO: 15.

17. The tetramer of claim 1, wherein the influenza NA head domain comprises the amino acid substitutions, numbered relative SEQ ID NO: 15, selected from:
a) I99P, C161V, E165S, S172A, S196T;
b) I99P, T131Q, C161V, E165S, S172A, S196T;
c) I99P, T131Q, C161V, E165S, S172A, V177I, S196T;
d) I99P, T131Q, C161V, E165S, S172A, S196T, V453T;
e) I99P, T131Q, C161V, E165S, S172A, V177I, S196T, V205I;
f) I99P, Y100L, T131Q, C161V, E165S, S172A, V177I, S196T, V205I, Q408M, R419V;
g) I99P, C161V, E165S, S172A, V177I, S196T;
h) I99P, C161V, S172A, S196T; or
i) I99P, C161V, S172A, V177I, S196T.

18. The tetramer of claim 1, wherein:
a) the NA head domains are in a closed conformation;
b) the tetramer has increased thermal stability in the presence of chelating agent compared to a tetramer with tetrabrachion tetramerization domain;
c) the tetramer has increased interaction to neuraminidase inhibitor compared to a tetramer with tetrabrachion tetramerization domain; and/or
d) the tetramer is expressed in recombinant cell culture at increased yield compared to a tetramer with a tetrabrachion tetramerization domain.

19. A fusion protein comprised by the tetramer of claim 1.

20. (canceled)

21. A polynucleotide encoding the fusion protein of claim 19, optionally wherein said polynucleotide is an mRNA.

22. (canceled)

23. A pharmaceutical composition, comprising the tetramer of claim 1 and a pharmaceutically acceptable carrier.

24. (canceled)

25. A method of immunizing a subject in need thereof against infection by influenza or of treating and/or preventing influenza in a subject in need thereof, the method comprising administering to the subject an effective amount of the pharmaceutical composition of claim 23.

26. (canceled)

27. The method of claim 25, wherein the method generates an antibody response to the tetramer equivalent to or greater than the antibody response to administration of a tetramer with a tetrabrachion tetramerization domain and/or at least as effectively as administering a tetramer with a tetrabrachion tetramerization domain.

28.-29. (canceled)

30. A host cell comprising the polynucleotide of claim 21.

31. A method of producing a recombinant influenza neuraminidase (NA) tetramer, comprising culturing the host cell of claim 30 in cell culture media, and harvesting the recombinant influenza neuraminidase (NA) tetramer from the cell culture media or from the host cell.

32.-33. (canceled)

34. A recombinant influenza neuraminidase (NA) polypeptide, comprising an influenza NA head domain comprising one or more amino acid substitutions selected from the group consisting of I99P, Y100L, T131Q, C161V, E165S, S172A, V177I, S196T, V205I, Q408M, R419V, and V453T, numbered relative SEQ ID NO: 15.

35.-53. (canceled)

54. A pharmaceutical composition, comprising a recombinant influenza neuraminidase (NA) tetramer according to claim 1 and an influenza HA vaccine antigen, wherein the influenza HA vaccine antigen is a protein nanostructure comprising an influenza HA ectodomain.

55. (canceled)

56. The pharmaceutical composition of claim 54, wherein the protein nanostructure comprises a trimeric component comprising a trimer protein comprising a fusion protein, wherein the fusion protein comprises, in N- to C-terminal order, the influenza HA ectodomain, a polypeptide linker, and an assembly domain.

57. (canceled)

58. The pharmaceutical composition of claim 56,
wherein the assembly domain comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 458-460;
wherein the influenza HA ectodomain comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 452-455, or 465;
wherein the pentamer protein comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 461-463;
wherein the trimer protein comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 448-451, or 464; and/or
wherein the polypeptide linker comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 456 or 457.

59.-63. (canceled)

64. The pharmaceutical composition of claim 56, wherein the composition comprises one or more pharmaceutically acceptable diluents, adjuvants, or excipients.

65.-69. (canceled)

70. A method of immunizing a subject in need thereof against infection by influenza or of treating and/or preventing influenza in a subject in need thereof, the method comprising administering to the subject an effective amount of the pharmaceutical composition according to claim 56.

71.-73. (canceled)

74. A protein nanostructure comprising a trimeric component comprising a trimer polypeptide and a pentameric component comprising a pentamer polypeptide, wherein the trimer polypeptide is a fusion protein comprising, in N- to C-terminal order, an influenza HA ectodomain, a polypeptide linker, and an assembly domain,
wherein the assembly domain comprises a polypeptide sequence at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 458-460;
wherein the influenza HA ectodomain comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 452-455, or 465;
wherein the pentamer protein comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 461-463;
wherein the trimer protein comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 448-451, or 464; and/or
wherein the polypeptide linker comprises a polypeptide sequence that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 456 or 457.

75.-81. (canceled)


SEQUENCE LISTING (495 sequences)

SEQ ID NO: 1 - protein, 50 aa, synthetic construct
VIYDVRDDVLTNLTITLDDIKTDLTTLINTRVDKLEMMINDNVTTILASG

SEQ ID NO: 2 - protein, 47 aa, synthetic construct
DVRDDVLTNLTITLDDIKTDLTTLINTRVDKLEMMINDNVTTILASG

SEQ ID NO: 3 - protein, 43 aa, synthetic construct
DVLTNLTITLDDIKTDLTTLINTRVDKLEMMINDNVTTILASG

SEQ ID NO: 4 - protein, 39 aa, synthetic construct
NLTITLDDIKTDLTTLINTRVDKLEMMINDNVTTILASG

SEQ ID NO: 5 - protein, 35 aa, synthetic construct
TLDDIKTDLTTLINTRVDKLEMMINDNVTTILASG

SEQ ID NO: 6 - protein, 50 aa, synthetic construct
IINETADDIVYRLTVIIDDRYESLKNLITLRADRLEMIINDNVSTILASG

SEQ ID NO: 7 - protein, 47 aa, synthetic construct
ETADDIVYRLTVIIDDRYESLKNLITLRADRLEMIINDNVSTILASG

SEQ ID NO: 8 - protein, 43 aa, synthetic construct
DIVYRLTVIIDDRYESLKNLITLRADRLEMIINDNVSTILASG

SEQ ID NO: 9 - protein, 39 aa, synthetic construct
RLTVIIDDRYESLKNLITLRADRLEMIINDNVSTILASG

SEQ ID NO: 10 - protein, 35 aa, synthetic construct
IIDDRYESLKNLITLRADRLEMIINDNVSTILASG

SEQ ID NO: 11 - protein, 8 aa, synthetic construct
GGSGGSGG

SEQ ID NO: 12 - protein, 6 aa, synthetic construct
ATPRGG

SEQ ID NO: 13 - protein, 8 aa, synthetic construct
ATPRGSGG

SEQ ID NO: 14 - protein, 10 aa, synthetic construct
GGATPRGSGG

SEQ ID NO: 15 - protein, 387 aa, synthetic construct [sequence omitted]

SEQ ID NO: 16 - protein, 387 aa, synthetic construct
VKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 17 - protein, 387 aa, synthetic construct
VKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 18 - protein, 387 aa, synthetic construct
VKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 19 - protein, 387 aa, synthetic construct
VKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 20 - protein, 387 aa, synthetic construct
VTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 21 - protein, 387 aa, synthetic construct
VILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAMTDRSGYSGSFVQHPELTGLDCMRPCFWVELIRGLPEEDAIWTSGSIISFCGVNGDTVDWSWPDGAELPFTIDK

SEQ ID NO: 22 - protein, 387 aa, synthetic construct [sequence omitted]

SEQ ID NO: 23 - protein, 387 aa, synthetic construct
EYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 24 - protein, 387 aa, synthetic construct
EYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 25 - protein, 387 aa, synthetic construct
EYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 26 - protein, 387 aa, synthetic construct
EYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 27 - protein, 391 aa, synthetic construct
PEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 28 - protein, 391 aa, synthetic construct
PEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 29 - protein, 391 aa, synthetic construct
PEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 30 - protein, 391 aa, synthetic construct
SEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 31 - protein, 391 aa, synthetic construct
PEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 32 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 33 - protein, 447 aa, synthetic construct
VIYDVRDDVLTNLTITLDDIKTDLTTLINTRVDKLEMMINDNVTTILASGSGAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 34 - protein, 445 aa, synthetic construct
VIYDVRDDVLTNLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 35 - protein, 443 aa, synthetic construct
VIYDVRDDVLTNLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 36 - protein, 441 aa, synthetic construct
NLTITLDDIKTDLTTLINTRVDKLEMMINDNVTTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 37 - protein, 436 aa, synthetic construct
NLTITLDDIKTDLTTLINTRVDKLEMMINDNVTTILASGSGAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 38 - protein, 434 aa, synthetic construct
NLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 39 - protein, 432 aa, synthetic construct
NLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 40 - protein, 437 aa, synthetic construct
TLDDIKTDLTTLINTRVDKLEMMINDNVTTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 41 - protein, 432 aa, synthetic construct
TLDDIKTDLTTLINTRVDKLEMMINDNVTTILASGSGAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 42 - protein, 430 aa, synthetic construct
TLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 43 - protein, 428 aa, synthetic construct
TLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 44 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 45 - protein, 447 aa, synthetic construct
VIYDVRDDVLTNLTITLDDIKTDLTTLINTRVDKLEMMINDNVTTILASGSGAAGQSVVSVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 46 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 47 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 48 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 49 - protein, 447 aa, synthetic construct
VIYDVRDDVLTNLTITLDDIKTDLTTLINTRVDKLEMMINDNVTTILASGSGAAGQSVVSVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 50 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 51 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 52 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 53 - protein, 447 aa, synthetic construct
VIYDVRDDVLTNLTITLDDIKTDLTTLINTRVDKLEMMINDNVTTILASGSGAAGQSVVSVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 54 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 55 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGINWLTIGITGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 56 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 57 - protein, 447 aa, synthetic construct
VIYDVRDDVLTNLTITLDDIKTDLTTLINTRVDKLEMMINDNVTTILASGSGAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 58 - protein, 445 aa, synthetic construct
VIYDVRDDVLTNLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 59 - protein, 443 aa, synthetic construct
VIYDVRDDVLTNLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 60 - protein, 441 aa, synthetic construct
NLTITLDDIKTDLTTLINTRVDKLEMMINDNVTTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 61 - protein, 436 aa, synthetic construct
NLTITLDDIKTDLTTLINTRVDKLEMMINDNVTTILASGSGAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 62 - protein, 434 aa, synthetic construct
NLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 63 - protein, 432 aa, synthetic construct
NLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 64 - protein, 437 aa, synthetic construct
TLDDIKTDLTTLINTRVDKLEMMINDNVTTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 65 - protein, 432 aa, synthetic construct
TLDDIKTDLTTLINTRVDKLEMMINDNVTTILASGSGAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 66 - protein, 430 aa, synthetic construct
TLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 67 - protein, 428 aa, synthetic construct
TLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 68 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 69 - protein, 447 aa, synthetic construct
VIYDVRDDVLTNLTITLDDIKTDLTTLINTRVDKLEMMINDNVTTILASGSGAAGQSVVSVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 70 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 71 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 72 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 73 - protein, 447 aa, synthetic construct
VIYDVRDDVLTNLTITLDDIKTDLTTLINTRVDKLEMMINDNVTTILASGSGAAGQSVVSVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 74 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 75 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 76 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 77 - protein, 447 aa, synthetic construct
VIYDVRDDVLTNLTITLDDIKTDLTTLINTRVDKLEMMINDNVTTILASGSGAAGQSVVSVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 78 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 79 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 80 - protein, 452 aa, synthetic construct
VIYDVRDDVLTNLTITLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 81 - protein, 447 aa, synthetic construct
VIYDVRDDVLTNLTITLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 82 - protein, 445 aa, synthetic construct
VIYDVRDDVLTNLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 83 - protein, 443 aa, synthetic construct
VIYDVRDDVLTNLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 84 - protein, 441 aa, synthetic construct
NLTITLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 85 - protein, 436 aa, synthetic construct
NLTITLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 86 - protein, 434 aa, synthetic construct
NLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 87 - protein, 432 aa, synthetic construct
NLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 88 - protein, 437 aa, synthetic construct
TLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 89 - protein, 432 aa, synthetic construct
TLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 90 - protein, 430 aa, synthetic construct
TLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 91 - protein, 428 aa, synthetic construct
TLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 92 - protein, 452 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 93 - protein, 447 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 94 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 95 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 96 - protein, 452 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 97 - protein, 447 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 98 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 99 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 100 - protein, 452 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 101 - protein, 447 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 102 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 103 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 104 - protein, 452 aa, synthetic construct
VIYDVRDDVLTNLTITLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 105 - protein, 447 aa, synthetic construct
VIYDVRDDVLTNLTITLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 106 - protein, 445 aa, synthetic construct
VIYDVRDDVLTNLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 107 - protein, 443 aa, synthetic construct
VIYDVRDDVLTNLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 108 - protein, 441 aa, synthetic construct
NLTITLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 109 - protein, 436 aa, synthetic construct
NLTITLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 110 - protein, 434 aa, synthetic construct
NLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 111 - protein, 432 aa, synthetic construct
NLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 112 - protein, 437 aa, synthetic construct
TLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 113 - protein, 432 aa, synthetic construct
TLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 114 - protein, 430 aa, synthetic construct
TLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 115 - protein, 428 aa, synthetic construct
TLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 116 - protein, 452 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 117 - protein, 447 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 118 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 119 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 120 - protein, 452 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 121 - protein, 447 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 122 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 123 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 124 - protein, 452 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 125 - protein, 447 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 126 - protein, 445 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 127 - protein, 443 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 128 - protein, 452 aa, synthetic construct
VIYDVRDDVLTNLTITLDDIKTDLTTLINTRVDKLEMMINDNVTTILASGSGSNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 129 - protein, 447 aa, synthetic construct
VIYDVRDDVLTNLTITLDDIKTDLTTLINTRVDKLEMMINDNVTTILASGSGLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 130 - protein, 445 aa, synthetic construct
VIYDVRDDVLTNLTITLDDIKTDLTTLINTRVDKLEMMINDNVTTILASGATPRGSGGVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 131 - protein, 443 aa, synthetic construct
VIYDVRDDVLTNLTITLDDIKTDLTTLINTRVDKLEMMINDNVTTILASGATPRGGVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 132 - protein, 441 aa, synthetic construct
NLTITLDDIKTDLTTLINTRVDKLEMMINDNVTTILASGSGSNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 133 - protein, 436 aa, synthetic construct
NLTITLDDIKTDLTTLINTRVDKLEMMINDNVTTILASGSGLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 134 - protein, 434 aa, synthetic construct
NLTITLDDIKTDLTTLINTRVDKLEMMINDNVTTILASGATPRGSGGVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 135 - protein, 432 aa, synthetic construct
NLTITLDDIKTDLTTLINTRVDKLEMMINDNVTTILASGATPRGGVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 136 - protein, 437 aa, synthetic construct
TLDDIKTDLTTLINTRVDKLEMMINDNVTTILASGSGSNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 137 - protein, 432 aa, synthetic construct
TLDDIKTDLTTLINTRVDKLEMMINDNVTTILASGSGLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 138 - protein, 430 aa, synthetic construct
TLDDIKTDLTTLINTRVDKLEMMINDNVTTILASGATPRGSGGVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 139 - protein, 428 aa, synthetic construct
TLDDIKTDLTTLINTRVDKLEMMINDNVTTILASGATPRGGVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 140 - protein, 452 aa, synthetic construct
VIYDVRDDVLTNLTITLDDIKTDLTTLINTRVDKLEMMINDNVTTILASGSGSNTNFLTEKAVASVKLAGNSSLCPINGWAPYSKDNSIRIGSKGDVFVIREPFISCSHLECRTFFLTQGALLNDKHSNGTVKDRSPHRTLMSVPVGSAPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 141 - protein, 447 aa, synthetic construct
VIYDVRDDVLTNLTITLDDIKTDLTTLINTRVDKLEMMINDNVTTILASGSGLTEKAVASVKLAGNSSLCPINGWAPYSKDNSIRIGSKGDVFVIREPFISCSHLECRTFFLTQGALLNDKHSNGTVKDRSPHRTLMSVPVGSAPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 142 - protein, 445 aa, synthetic construct
VIYDVRDDVLTNLTITLDDIKTDLTTLINTRVDKLEMMINDNVTTILASGATPRGSGGVKLAGNSSLCPINGWAPYSKDNSIRIGSKGDVFVIREPFISCSHLECRTFFLTQGALLNDKHSNGTVKDRSPHRTLMSVPVGSAPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 143 - protein, 443 aa, synthetic construct
VIYDVRDDVLTNLTITLDDIKTDLTTLINTRVDKLEMMINDNVTTILASGATPRGGVKLAGNSSLCPINGWAPYSKDNSIRIGSKGDVFVIREPFISCSHLECRTFFLTQGALLNDKHSNGTVKDRSPHRTLMSVPVGSAPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 144 - protein, 452 aa, synthetic construct
VIYDVRDDVLTNLTITLDDIKTDLTTLINTRVDKLEMMINDNVTTILASGSGSNTNFLTEKAVASVKLAGNSSLCPINGWAPYSKDNSIRIGSKGDVFVIREPFISCSHLECRTFFLTQGALLNDKHSNGTVKDRSPHRTLMSVPVGSAPSPYNARFESIAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 145 - protein, 447 aa, synthetic construct
VIYDVRDDVLTNLTITLDDIKTDLTTLINTRVDKLEMMINDNVTTILASGSGLTEKAVASVKLAGNSSLCPINGWAPYSKDNSIRIGSKGDVFVIREPFISCSHLECRTFFLTQGALLNDKHSNGTVKDRSPHRTLMSVPVGSAPSPYNARFESIAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 146 - protein, 445 aa, synthetic construct
VIYDVRDDVLTNLTITLDDIKTDLTTLINTRVDKLEMMINDNVTTILASGATPRGSGGVKLAGNSSLCPINGWAPYSKDNSIRIGSKGDVFVIREPFISCSHLECRTFFLTQGALLNDKHSNGTVKDRSPHRTLMSVPVGSAPSPYNARFESIAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 147 - protein, 443 aa, synthetic construct
VIYDVRDDVLTNLTITLDDIKTDLTTLINTRVDKLEMMINDNVTTILASGATPRGGVKLAGNSSLCPINGWAPYSKDNSIRIGSKGDVFVIREPFISCSHLECRTFFLTQGALLNDKHSNGTVKDRSPHRTLMSVPVGSAPSPYNARFESIAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 148 - protein, 452 aa, synthetic construct
VIYDVRDDVLTNLTITLDDIKTDLTTLINTRVDKLEMMINDNVTTILASGSGSNTNFLTEKAVASVKLAGNSSLCPINGWAPYSKDNSIRIGSKGDVFVIREPFISCSHLECRTFFLTQGALLNDKHSNGTVKDRSPHRTLMSVPVGEAPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 149 - protein, 447 aa, synthetic construct
VIYDVRDDVLTNLTITLDDIKTDLTTLINTRVDKLEMMINDNVTTILASGSGLTEKAVASVKLAGNSSLCPINGWAPYSKDNSIRIGSKGDVFVIREPFISCSHLECRTFFLTQGALLNDKHSNGTVKDRSPHRTLMSVPVGEAPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 150 - protein, 445 aa, synthetic construct
VIYDVRDDVLTNLTITLDDIKTDLTTLINTRVDKLEMMINDNVTTILASGATPRGSGGVKLAGNSSLCPINGWAPYSKDNSIRIGSKGDVFVIREPFISCSHLECRTFFLTQGALLNDKHSNGTVKDRSPHRTLMSVPVGEAPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 151 - protein, 443 aa, synthetic construct
VIYDVRDDVLTNLTITLDDIKTDLTTLINTRVDKLEMMINDNVTTILASGATPRGGVKLAGNSSLCPINGWAPYSKDNSIRIGSKGDVFVIREPFISCSHLECRTFFLTQGALLNDKHSNGTVKDRSPHRTLMSVPVGEAPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 152 - protein, 452 aa, synthetic construct
VIYDVRDDVLTNLTITLDDIKTDLTTLINTRVDKLEMMINDNVTTILASGSGSNTNFLTEKAVASVKLAGNSSLCPINGWAIYSKDNSVRIGSKGDVFVIREPFISCSHLECRTFFLTQGALLNDKHSNGTVKDRSPHRTLMSCPIGEVPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 153 - protein, 447 aa, synthetic construct
VIYDVRDDVLTNLTITLDDIKTDLTTLINTRVDKLEMMINDNVTTILASGSGLTEKAVASVKLAGNSSLCPINGWAIYSKDNSVRIGSKGDVFVIREPFISCSHLECRTFFLTQGALLNDKHSNGTVKDRSPHRTLMSCPIGEVPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 154 - protein, 445 aa, synthetic construct
VIYDVRDDVLTNLTITLDDIKTDLTTLINTRVDKLEMMINDNVTTILASGATPRGSGGVKLAGNSSLCPINGWAIYSKDNSVRIGSKGDVFVIREPFISCSHLECRTFFLTQGALLNDKHSNGTVKDRSPHRTLMSCPIGEVPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 155 - protein, 443 aa, synthetic construct
VIYDVRDDVLTNLTITLDDIKTDLTTLINTRVDKLEMMINDNVTTILASGATPRGGVKLAGNSSLCPINGWAIYSKDNSVRIGSKGDVFVIREPFISCSHLECRTFFLTQGALLNDKHSNGTVKDRSPHRTLMSCPIGEVPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 156 - protein, 452 aa, synthetic construct
VIYDVRDDVLTNLTITLDDIKTDLTTLINTRVDKLEMMINDNVTTILASGSGSNTNFLTEKAVASVKLAGNSSLCPINGWAPYSKDNSVRIGSKGDVFVIREPFISCSHLECRTFFLTQGALLNDKHSNGTVKDRSPHRTLMSVPIGSVPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 157 - protein, 447 aa, synthetic construct
VIYDVRDDVLTNLTITLDDIKTDLTTLINTRVDKLEMMINDNVTTILASGSGLTEKAVASVKLAGNSSLCPINGWAPYSKDNSVRIGSKGDVFVIREPFISCSHLECRTFFLTQGALLNDKHSNGTVKDRSPHRTLMSVPIGSVPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 158 - protein, 445 aa, synthetic construct
VIYDVRDDVLTNLTITLDDIKTDLTTLINTRVDKLEMMINDNVTTILASGATPRGSGGVKLAGNSSLCPINGWAPYSKDNSVRIGSKGDVFVIREPFISCSHLECRTFFLTQGALLNDKHSNGTVKDRSPHRTLMSVPIGSVPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 159 - protein, 443 aa, synthetic construct
VIYDVRDDVLTNLTITLDDIKTDLTTLINTRVDKLEMMINDNVTTILASGATPRGGVKLAGNSSLCPINGWAPYSKDNSVRIGSKGDVFVIREPFISCSHLECRTFFLTQGALLNDKHSNGTVKDRSPHRTLMSVPIGSVPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 160 - protein, 452 aa, synthetic construct
VIYDVRDDVLTNLTITLDDIKTDLTTLINTRVDKLEMMINDNVTTILASGSGSNTNFLTEKAVASVKLAGNSSLCPINGWAPYSKDNSVRIGSKGDVFVIREPFISCSHLECRTFFLTQGALLNDKHSNGTVKDRSPHRTLMSVPIGSVPSPYNARFESIAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 161 - protein, 447 aa, synthetic construct
VIYDVRDDVLTNLTITLDDIKTDLTTLINTRVDKLEMMINDNVTTILASGSGLTEKAVASVKLAGNSSLCPINGWAPYSKDNSVRIGSKGDVFVIREPFISCSHLECRTFFLTQGALLNDKHSNGTVKDRSPHRTLMSVPIGSVPSPYNARFESIAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 162 - protein, 445 aa, synthetic construct
VIYDVRDDVLTNLTITLDDIKTDLTTLINTRVDKLEMMINDNVTTILASGATPRGSGGVKLAGNSSLCPINGWAPYSKDNSVRIGSKGDVFVIREPFISCSHLECRTFFLTQGALLNDKHSNGTVKDRSPHRTLMSVPIGSVPSPYNARFESIAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 163 - protein, 443 aa, synthetic construct
VIYDVRDDVLTNLTITLDDIKTDLTTLINTRVDKLEMMINDNVTTILASGATPRGGVKLAGNSSLCPINGWAPYSKDNSVRIGSKGDVFVIREPFISCSHLECRTFFLTQGALLNDKHSNGTVKDRSPHRTLMSVPIGSVPSPYNARFESIAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 164 - protein, 452 aa, synthetic construct
VIYDVRDDVLTNLTITLDDIKTDLTTLINTRVDKLEMMINDNVTTILASGSGSNTNFLTEKAVASVKLAGNSSLCPINGWAPYSKDNSVRIGSKGDVFVIREPFISCSHLECRTFFLTQGALLNDKHSNGTVKDRSPHRTLMSVPIGEVPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 165 - protein, 447 aa, synthetic construct
VIYDVRDDVLTNLTITLDDIKTDLTTLINTRVDKLEMMINDNVTTILASGSGLTEKAVASVKLAGNSSLCPINGWAPYSKDNSVRIGSKGDVFVIREPFISCSHLECRTFFLTQGALLNDKHSNGTVKDRSPHRTLMSVPIGEVPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 166 - protein, 445 aa, synthetic construct
VIYDVRDDVLTNLTITLDDIKTDLTTLINTRVDKLEMMINDNVTTILASGATPRGSGGVKLAGNSSLCPINGWAPYSKDNSVRIGSKGDVFVIREPFISCSHLECRTFFLTQGALLNDKHSNGTVKDRSPHRTLMSVPIGEVPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 167 - protein, 443 aa, synthetic construct
VIYDVRDDVLTNLTITLDDIKTDLTTLINTRVDKLEMMINDNVTTILASGATPRGGVKLAGNSSLCPINGWAPYSKDNSVRIGSKGDVFVIREPFISCSHLECRTFFLTQGALLNDKHSNGTVKDRSPHRTLMSVPIGEVPSPYNARFESVAWSASACHDGTSWLTIGITGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 168 - protein, 452 aa, synthetic construct
VIYDVRDDVLTNLTITLDDIKTDLTTLINTRVDKLEMMINDNVTTILASGSGSNTNFLAEQAVTSVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 169 - protein, 447 aa, synthetic construct
VIYDVRDDVLTNLTITLDDIKTDLTTLINTRVDKLEMMINDNVTTILASGSGLAEQAVTSVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 170 - protein, 445 aa, synthetic construct
VIYDVRDDVLTNLTITLDDIKTDLTTLINTRVDKLEMMINDNVTTILASGATPRGSGGVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 171 - protein, 443 aa, synthetic construct
VIYDVRDDVLTNLTITLDDIKTDLTTLINTRVDKLEMMINDNVTTILASGATPRGGVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 172 - protein, 441 aa, synthetic construct
NLTITLDDIKTDLTTLINTRVDKLEMMINDNVTTILASGSGSNTNFLAEQAVTSVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 173 - protein, 436 aa, synthetic construct
NLTITLDDIKTDLTTLINTRVDKLEMMINDNVTTILASGSGLAEQAVTSVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 174 - protein, 434 aa, synthetic construct
NLTITLDDIKTDLTTLINTRVDKLEMMINDNVTTILASGATPRGSGGVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 175 - protein, 432 aa, synthetic construct
NLTITLDDIKTDLTTLINTRVDKLEMMINDNVTTILASGATPRGGVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 176 - protein, 437 aa, synthetic construct
TLDDIKTDLTTLINTRVDKLEMMINDNVTTILASGSGSNTNFLAEQAVTSVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 177 - protein, 432 aa, synthetic construct
TLDDIKTDLTTLINTRVDKLEMMINDNVTTILASGSGLAEQAVTSVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 178 - protein, 430 aa, synthetic construct
TLDDIKTDLTTLINTRVDKLEMMINDNVTTILASGATPRGSGGVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 179 - protein, 428 aa, synthetic construct
TLDDIKTDLTTLINTRVDKLEMMINDNVTTILASGATPRGGVTLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 180 - protein, 452 aa, synthetic construct
VIYDVRDDVLTNLTITLDDIKTDLTTLINTRVDKLEMMINDNVTTILASGSGSNTNFLAEQAVTSVTLAGNSSLCPISGWAPYSKDNGIRIGSKGDVFVIREPFISCSHLECRTFFLTQGALLNDKHSNGTVKDRSPYRTLMSVPVGSAPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 181 - protein, 447 aa, synthetic construct
VIYDVRDDVLTNLTITLDDIKTDLTTLINTRVDKLEMMINDNVTTILASGSGLAEQAVTSVTLAGNSSLCPISGWAPYSKDNGIRIGSKGDVFVIREPFISCSHLECRTFFLTQGALLNDKHSNGTVKDRSPYRTLMSVPVGSAPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 182 - protein, 445 aa, synthetic construct
VIYDVRDDVLTNLTITLDDIKTDLTTLINTRVDKLEMMINDNVTTILASGATPRGSGGVTLAGNSSLCPISGWAPYSKDNGIRIGSKGDVFVIREPFISCSHLECRTFFLTQGALLNDKHSNGTVKDRSPYRTLMSVPVGSAPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 183 - protein, 443 aa, synthetic construct
VIYDVRDDVLTNLTITLDDIKTDLTTLINTRVDKLEMMINDNVTTILASGATPRGGVTLAGNSSLCPISGWAPYSKDNGIRIGSKGDVFVIREPFISCSHLECRTFFLTQGALLNDKHSNGTVKDRSPYRTLMSVPVGSAPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 184 - protein, 452 aa, synthetic construct
VIYDVRDDVLTNLTITLDDIKTDLTTLINTRVDKLEMMINDNVTTILASGSGSNTNFLAEQAVTSVTLAGNSSLCPISGWAPYSKDNGIRIGSKGDVFVIREPFISCSHLECRTFFLTQGALLNDKHSNGTVKDRSPYRTLMSVPVGSAPSPYNARFESIAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 185 - protein, 447 aa, synthetic construct
VIYDVRDDVLTNLTITLDDIKTDLTTLINTRVDKLEMMINDNVTTILASGSGLAEQAVTSVTLAGNSSLCPISGWAPYSKDNGIRIGSKGDVFVIREPFISCSHLECRTFFLTQGALLNDKHSNGTVKDRSPYRTLMSVPVGSAPSPYNARFESIAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 186 - protein, 445 aa, synthetic construct
VIYDVRDDVLTNLTITLDDIKTDLTTLINTRVDKLEMMINDNVTTILASGATPRGSGGVTLAGNSSLCPISGWAPYSKDNGIRIGSKGDVFVIREPFISCSHLECRTFFLTQGALLNDKHSNGTVKDRSPYRTLMSVPVGSAPSPYNARFESIAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 187 - protein, 443 aa, synthetic construct
VIYDVRDDVLTNLTITLDDIKTDLTTLINTRVDKLEMMINDNVTTILASGATPRGGVTLAGNSSLCPISGWAPYSKDNGIRIGSKGDVFVIREPFISCSHLECRTFFLTQGALLNDKHSNGTVKDRSPYRTLMSVPVGSAPSPYNARFESIAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 188 - protein, 452 aa, synthetic construct
VIYDVRDDVLTNLTITLDDIKTDLTTLINTRVDKLEMMINDNVTTILASGSGSNTNFLAEQAVTSVTLAGNSSLCPISGWAPYSKDNGIRIGSKGDVFVIREPFISCSHLECRTFFLTQGALLNDKHSNGTVKDRSPYRTLMSVPVGEAPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 189 - protein, 447 aa, synthetic construct
VIYDVRDDVLTNLTITLDDIKTDLTTLINTRVDKLEMMINDNVTTILASGSGLAEQAVTSVTLAGNSSLCPISGWAPYSKDNGIRIGSKGDVFVIREPFISCSHLECRTFFLTQGALLNDKHSNGTVKDRSPYRTLMSVPVGEAPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 190 - protein, 445 aa, synthetic construct
VIYDVRDDVLTNLTITLDDIKTDLTTLINTRVDKLEMMINDNVTTILASGATPRGSGGVTLAGNSSLCPISGWAPYSKDNGIRIGSKGDVFVIREPFISCSHLECRTFFLTQGALLNDKHSNGTVKDRSPYRTLMSVPVGEAPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 191 - protein, 443 aa, synthetic construct
VIYDVRDDVLTNLTITLDDIKTDLTTLINTRVDKLEMMINDNVTTILASGATPRGGVTLAGNSSLCPISGWAPYSKDNGIRIGSKGDVFVIREPFISCSHLECRTFFLTQGALLNDKHSNGTVKDRSPYRTLMSVPVGEAPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 192 - protein, 452 aa, synthetic construct
VIYDVRDDVLTNLTITLDDIKTDLTTLINTRVDKLEMMINDNVTTILASGSGSNTNFLAEQAVTSVTLAGNSSLCPISGWAIYSKDNSVRIGSKGDVFVIREPFISCSHLECRTFFLTQGALLNDKHSNGTVKDRSPYRTLMSCPIGEVPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 193 - protein, 447 aa, synthetic construct
VIYDVRDDVLTNLTITLDDIKTDLTTLINTRVDKLEMMINDNVTTILASGSGLAEQAVTSVTLAGNSSLCPISGWAIYSKDNSVRIGSKGDVFVIREPFISCSHLECRTFFLTQGALLNDKHSNGTVKDRSPYRTLMSCPIGEVPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 194 - protein, 445 aa, synthetic construct
VIYDVRDDVLTNLTITLDDIKTDLTTLINTRVDKLEMMINDNVTTILASGATPRGSGGVTLAGNSSLCPISGWAIYSKDNSVRIGSKGDVFVIREPFISCSHLECRTFFLTQGALLNDKHSNGTVKDRSPYRTLMSCPIGEVPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 195 - protein, 443 aa, synthetic construct
VIYDVRDDVLTNLTITLDDIKTDLTTLINTRVDKLEMMINDNVTTILASGATPRGGVTLAGNSSLCPISGWAIYSKDNSVRIGSKGDVFVIREPFISCSHLECRTFFLTQGALLNDKHSNGTVKDRSPYRTLMSCPIGEVPSPYNSRFESVAWSASACHDGISWLTIGISGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 196 - protein, 452 aa, synthetic construct
VIYDVRDDVLTNLTITLDDIKTDLTTLINTRVDKLEMMINDNVTTILASGSGSNTNFLAEQAVTSVTLAGNSSLCPISGWAPYSKDNSVRIGSKGDVFVIREPFISCSHLECRTFFLTQGALLNDKHSNGTVKDRSPYRTLMSVPIGSVPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 197 - protein, 447 aa, synthetic construct
VIYDVRDDVLTNLTITLDDIKTDLTTLINTRVDKLEMMINDNVTTILASGSGLAEQAVTSVTLAGNSSLCPISGWAPYSKDNSVRIGSKGDVFVIREPFISCSHLECRTFFLTQGALLNDKHSNGTVKDRSPYRTLMSVPIGSVPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 198 - protein, 445 aa, synthetic construct
VIYDVRDDVLTNLTITLDDIKTDLTTLINTRVDKLEMMINDNVTTILASGATPRGSGGVTLAGNSSLCPISGWAPYSKDNSVRIGSKGDVFVIREPFISCSHLECRTFFLTQGALLNDKHSNGTVKDRSPYRTLMSVPIGSVPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 199 - protein, 443 aa, synthetic construct
VIYDVRDDVLTNLTITLDDIKTDLTTLINTRVDKLEMMINDNVTTILASGATPRGGVTLAGNSSLCPISGWAPYSKDNSVRIGSKGDVFVIREPFISCSHLECRTFFLTQGALLNDKHSNGTVKDRSPYRTLMSVPIGSVPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 200 - protein, 452 aa, synthetic construct
VIYDVRDDVLTNLTITLDDIKTDLTTLINTRVDKLEMMINDNVTTILASGSGSNTNFLAEQAVTSVTLAGNSSLCPISGWAPYSKDNSVRIGSKGDVFVIREPFISCSHLECRTFFLTQGALLNDKHSNGTVKDRSPYRTLMSVPIGSVPSPYNARFESIAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 201 - protein, 447 aa, synthetic construct
VIYDVRDDVLTNLTITLDDIKTDLTTLINTRVDKLEMMINDNVTTILASGSGLAEQAVTSVTLAGNSSLCPISGWAPYSKDNSVRIGSKGDVFVIREPFISCSHLECRTFFLTQGALLNDKHSNGTVKDRSPYRTLMSVPIGSVPSPYNARFESIAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 202 - protein, 445 aa, synthetic construct
VIYDVRDDVLTNLTITLDDIKTDLTTLINTRVDKLEMMINDNVTTILASGATPRGSGGVTLAGNSSLCPISGWAPYSKDNSVRIGSKGDVFVIREPFISCSHLECRTFFLTQGALLNDKHSNGTVKDRSPYRTLMSVPIGSVPSPYNARFESIAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 203 - protein, 443 aa, synthetic construct
VIYDVRDDVLTNLTITLDDIKTDLTTLINTRVDKLEMMINDNVTTILASGATPRGGVTLAGNSSLCPISGWAPYSKDNSVRIGSKGDVFVIREPFISCSHLECRTFFLTQGALLNDKHSNGTVKDRSPYRTLMSVPIGSVPSPYNARFESIAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 204 - protein, 452 aa, synthetic construct
VIYDVRDDVLTNLTITLDDIKTDLTTLINTRVDKLEMMINDNVTTILASGSGSNTNFLAEQAVTSVTLAGNSSLCPISGWAPYSKDNSVRIGSKGDVFVIREPFISCSHLECRTFFLTQGALLNDKHSNGTVKDRSPYRTLMSVPIGEVPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 205 - protein, 447 aa, synthetic construct
VIYDVRDDVLTNLTITLDDIKTDLTTLINTRVDKLEMMINDNVTTILASGSGLAEQAVTSVTLAGNSSLCPISGWAPYSKDNSVRIGSKGDVFVIREPFISCSHLECRTFFLTQGALLNDKHSNGTVKDRSPYRTLMSVPIGEVPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 206 - protein, 445 aa, synthetic construct
VIYDVRDDVLTNLTITLDDIKTDLTTLINTRVDKLEMMINDNVTTILASGATPRGSGGVTLAGNSSLCPISGWAPYSKDNSVRIGSKGDVFVIREPFISCSHLECRTFFLTQGALLNDKHSNGTVKDRSPYRTLMSVPIGEVPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 207 - protein, 443 aa, synthetic construct
VIYDVRDDVLTNLTITLDDIKTDLTTLINTRVDKLEMMINDNVTTILASGATPRGGVTLAGNSSLCPISGWAPYSKDNSVRIGSKGDVFVIREPFISCSHLECRTFFLTQGALLNDKHSNGTVKDRSPYRTLMSVPIGEVPSPYNARFESVAWSASACHDGISWLTIGITGPDNGAVAVLKYNGIITDTIKSWRNNILRTQESECACVNGSCFTVMTDGPSNGQASYKIFKIEKGKVVKSVELNAPNYHYEECSCYPDAGDIMCVCRDNWHGSNRPWVSFNQNLEYQIGYICSGVFGDNPRPNDRTGSCSPMSSNGAYGVKGFSFKYGNGVWIGRTKSTSSRSGFEMIWDPNGWTETDSSFSVKQDIVEITDWSGYSGSFVQHPELTGLDCMRPCFWVELIRGRPKENTIWTSGSSISFCGVNSDTVGWSWPDGAELPFTIDK

SEQ ID NO: 208 - protein, 452 aa, synthetic construct
VIYDVRDDVLTNLTITLDDIKTDLTTLINTRVDKLEMMINDNVTTILASGSGITYKVVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 209 - protein, 447 aa, synthetic construct
VIYDVRDDVLTNLTITLDDIKTDLTTLINTRVDKLEMMINDNVTTILASGSGVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 210 - protein, 445 aa, synthetic construct
VIYDVRDDVLTNLTITLDDIKTDLTTLINTRVDKLEMMINDNVTTILASGATPRGSGGVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 211 - protein, 443 aa, synthetic construct
VIYDVRDDVLTNLTITLDDIKTDLTTLINTRVDKLEMMINDNVTTILASGATPRGGVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 212 - protein, 441 aa, synthetic construct
NLTITLDDIKTDLTTLINTRVDKLEMMINDNVTTILASGSGITYKVVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 213 - protein, 436 aa, synthetic construct
NLTITLDDIKTDLTTLINTRVDKLEMMINDNVTTILASGSGVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 214 - protein, 434 aa, synthetic construct
NLTITLDDIKTDLTTLINTRVDKLEMMINDNVTTILASGATPRGSGGVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 215 - protein, 432 aa, synthetic construct
NLTITLDDIKTDLTTLINTRVDKLEMMINDNVTTILASGATPRGGVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 216 - protein, 437 aa, synthetic construct
TLDDIKTDLTTLINTRVDKLEMMINDNVTTILASGSGITYKVVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 217 - protein, 432 aa, synthetic construct
TLDDIKTDLTTLINTRVDKLEMMINDNVTTILASGSGVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 218 - protein, 430 aa, synthetic construct
TLDDIKTDLTTLINTRVDKLEMMINDNVTTILASGATPRGSGGVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 219 - protein, 428 aa, synthetic construct
TLDDIKTDLTTLINTRVDKLEMMINDNVTTILASGATPRGGVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 220 - protein, 452 aa, synthetic construct
VIYDVRDDVLTNLTITLDDIKTDLTTLINTRVDKLEMMINDNVTTILASGSGITYKVVAGQDSTSVILTGNSSLCPIRGWAPHSKDNGIRIGSKGDVFVIREPFISCSHLECRTFFLTQGALLNDKHSRGTFKDRSPYRALMSVPVGSAPSPYNARFESVAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 221 - protein, 447 aa, synthetic construct
VIYDVRDDVLTNLTITLDDIKTDLTTLINTRVDKLEMMINDNVTTILASGSGVAGQDSTSVILTGNSSLCPIRGWAPHSKDNGIRIGSKGDVFVIREPFISCSHLECRTFFLTQGALLNDKHSRGTFKDRSPYRALMSVPVGSAPSPYNARFESVAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 222 - protein, 445 aa, synthetic construct
VIYDVRDDVLTNLTITLDDIKTDLTTLINTRVDKLEMMINDNVTTILASGATPRGSGGVILTGNSSLCPIRGWAPHSKDNGIRIGSKGDVFVIREPFISCSHLECRTFFLTQGALLNDKHSRGTFKDRSPYRALMSVPVGSAPSPYNARFESVAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 223 - protein, 443 aa, synthetic construct
VIYDVRDDVLTNLTITLDDIKTDLTTLINTRVDKLEMMINDNVTTILASGATPRGGVILTGNSSLCPIRGWAPHSKDNGIRIGSKGDVFVIREPFISCSHLECRTFFLTQGALLNDKHSRGTFKDRSPYRALMSVPVGSAPSPYNARFESVAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 224 - protein, 452 aa, synthetic construct
VIYDVRDDVLTNLTITLDDIKTDLTTLINTRVDKLEMMINDNVTTILASGSGITYKVVAGQDSTSVILTGNSSLCPIRGWAPHSKDNGIRIGSKGDVFVIREPFISCSHLECRTFFLTQGALLNDKHSRGTFKDRSPYRALMSVPVGSAPSPYNARFESIAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 225 - protein, 447 aa, synthetic construct
VIYDVRDDVLTNLTITLDDIKTDLTTLINTRVDKLEMMINDNVTTILASGSGVAGQDSTSVILTGNSSLCPIRGWAPHSKDNGIRIGSKGDVFVIREPFISCSHLECRTFFLTQGALLNDKHSRGTFKDRSPYRALMSVPVGSAPSPYNARFESIAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 226 - protein, 445 aa, synthetic construct
VIYDVRDDVLTNLTITLDDIKTDLTTLINTRVDKLEMMINDNVTTILASGATPRGSGGVILTGNSSLCPIRGWAPHSKDNGIRIGSKGDVFVIREPFISCSHLECRTFFLTQGALLNDKHSRGTFKDRSPYRALMSVPVGSAPSPYNARFESIAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 227 - protein, 443 aa, synthetic construct
VIYDVRDDVLTNLTITLDDIKTDLTTLINTRVDKLEMMINDNVTTILASGATPRGGVILTGNSSLCPIRGWAPHSKDNGIRIGSKGDVFVIREPFISCSHLECRTFFLTQGALLNDKHSRGTFKDRSPYRALMSVPVGSAPSPYNARFESIAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 228 - protein, 452 aa, synthetic construct
VIYDVRDDVLTNLTITLDDIKTDLTTLINTRVDKLEMMINDNVTTILASGSGITYKVVAGQDSTSVILTGNSSLCPIRGWAPHSKDNGIRIGSKGDVFVIREPFISCSHLECRTFFLTQGALLNDKHSRGTFKDRSPYRALMSVPVGEAPSPYNARFESVAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 229 - protein, 447 aa, synthetic construct
VIYDVRDDVLTNLTITLDDIKTDLTTLINTRVDKLEMMINDNVTTILASGSGVAGQDSTSVILTGNSSLCPIRGWAPHSKDNGIRIGSKGDVFVIREPFISCSHLECRTFFLTQGALLNDKHSRGTFKDRSPYRALMSVPVGEAPSPYNARFESVAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 230 - protein, 445 aa, synthetic construct
VIYDVRDDVLTNLTITLDDIKTDLTTLINTRVDKLEMMINDNVTTILASGATPRGSGGVILTGNSSLCPIRGWAPHSKDNGIRIGSKGDVFVIREPFISCSHLECRTFFLTQGALLNDKHSRGTFKDRSPYRALMSVPVGEAPSPYNARFESVAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 231 - protein, 443 aa, synthetic construct
VIYDVRDDVLTNLTITLDDIKTDLTTLINTRVDKLEMMINDNVTTILASGATPRGGVILTGNSSLCPIRGWAPHSKDNGIRIGSKGDVFVIREPFISCSHLECRTFFLTQGALLNDKHSRGTFKDRSPYRALMSVPVGEAPSPYNARFESVAWSASACHDGMGWLTIGITGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 232 - protein, 452 aa, synthetic construct
VIYDVRDDVLTNLTITLDDIKTDLTTLINTRVDKLEMMINDNVTTILASGSGITYKVVAGQDSTSVILTGNSSLCPIRGWAIHSKDNSVRIGSKGDVFVIREPFISCSHLECRTFFLTQGALLNDKHSRGTFKDRSPYRTLMSCPIGEVPSPYNSRFESVAWSASACHDGMGWLTIGISGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 233 - protein, 447 aa, synthetic construct
VIYDVRDDVLTNLTITLDDIKTDLTTLINTRVDKLEMMINDNVTTILASGSGVAGQDSTSVILTGNSSLCPIRGWAIHSKDNSVRIGSKGDVFVIREPFISCSHLECRTFFLTQGALLNDKHSRGTFKDRSPYRTLMSCPIGEVPSPYNSRFESVAWSASACHDGMGWLTIGISGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 234 - protein, 445 aa, synthetic construct
VIYDVRDDVLTNLTITLDDIKTDLTTLINTRVDKLEMMINDNVTTILASGATPRGSGGVILTGNSSLCPIRGWAIHSKDNSVRIGSKGDVFVIREPFISCSHLECRTFFLTQGALLNDKHSRGTFKDRSPYRTLMSCPIGEVPSPYNSRFESVAWSASACHDGMGWLTIGISGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 235 - protein, 443 aa, synthetic construct
VIYDVRDDVLTNLTITLDDIKTDLTTLINTRVDKLEMMINDNVTTILASGATPRGGVILTGNSSLCPIRGWAIHSKDNSVRIGSKGDVFVIREPFISCSHLECRTFFLTQGALLNDKHSRGTFKDRSPYRTLMSCPIGEVPSPYNSRFESVAWSASACHDGMGWLTIGISGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 236 - protein, 452 aa, synthetic construct
VIYDVRDDVLTNLTITLDDIKTDLTTLINTRVDKLEMMINDNVTTILASGSGITYKVVAGQDSTSVILTGNSSLCPIRGWAPHSKDNSVRIGSKGDVFVIREPFISCSHLECRTFFLTQGALLNDKHSRGTFKDRSPYRTLMSVPIGSVPSPYNARFESVAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 237 - protein, 447 aa, synthetic construct
VIYDVRDDVLTNLTITLDDIKTDLTTLINTRVDKLEMMINDNVTTILASGSGVAGQDSTSVILTGNSSLCPIRGWAPHSKDNSVRIGSKGDVFVIREPFISCSHLECRTFFLTQGALLNDKHSRGTFKDRSPYRTLMSVPIGSVPSPYNARFESVAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 238 - protein, 445 aa, synthetic construct
VIYDVRDDVLTNLTITLDDIKTDLTTLINTRVDKLEMMINDNVTTILASGATPRGSGGVILTGNSSLCPIRGWAPHSKDNSVRIGSKGDVFVIREPFISCSHLECRTFFLTQGALLNDKHSRGTFKDRSPYRTLMSVPIGSVPSPYNARFESVAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 239 - protein, 443 aa, synthetic construct
VIYDVRDDVLTNLTITLDDIKTDLTTLINTRVDKLEMMINDNVTTILASGATPRGGVILTGNSSLCPIRGWAPHSKDNSVRIGSKGDVFVIREPFISCSHLECRTFFLTQGALLNDKHSRGTFKDRSPYRTLMSVPIGSVPSPYNARFESVAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 240 - protein, 452 aa, synthetic construct
VIYDVRDDVLTNLTITLDDIKTDLTTLINTRVDKLEMMINDNVTTILASGSGITYKVVAGQDSTSVILTGNSSLCPIRGWAPHSKDNSVRIGSKGDVFVIREPFISCSHLECRTFFLTQGALLNDKHSRGTFKDRSPYRTLMSVPIGSVPSPYNARFESIAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 241 - protein, 447 aa, synthetic construct
VIYDVRDDVLTNLTITLDDIKTDLTTLINTRVDKLEMMINDNVTTILASGSGVAGQDSTSVILTGNSSLCPIRGWAPHSKDNSVRIGSKGDVFVIREPFISCSHLECRTFFLTQGALLNDKHSRGTFKDRSPYRTLMSVPIGSVPSPYNARFESIAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 242 - protein, 445 aa, synthetic construct
VIYDVRDDVLTNLTITLDDIKTDLTTLINTRVDKLEMMINDNVTTILASGATPRGSGGVILTGNSSLCPIRGWAPHSKDNSVRIGSKGDVFVIREPFISCSHLECRTFFLTQGALLNDKHSRGTFKDRSPYRTLMSVPIGSVPSPYNARFESIAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 243 - protein, 443 aa, synthetic construct
VIYDVRDDVLTNLTITLDDIKTDLTTLINTRVDKLEMMINDNVTTILASGATPRGGVILTGNSSLCPIRGWAPHSKDNSVRIGSKGDVFVIREPFISCSHLECRTFFLTQGALLNDKHSRGTFKDRSPYRTLMSVPIGSVPSPYNARFESIAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 244 - protein, 452 aa, synthetic construct
VIYDVRDDVLTNLTITLDDIKTDLTTLINTRVDKLEMMINDNVTTILASGSGITYKVVAGQDSTSVILTGNSSLCPIRGWAPHSKDNSVRIGSKGDVFVIREPFISCSHLECRTFFLTQGALLNDKHSRGTFKDRSPYRTLMSVPIGEVPSPYNARFESVAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 245 - protein, 447 aa, synthetic construct
VIYDVRDDVLTNLTITLDDIKTDLTTLINTRVDKLEMMINDNVTTILASGSGVAGQDSTSVILTGNSSLCPIRGWAPHSKDNSVRIGSKGDVFVIREPFISCSHLECRTFFLTQGALLNDKHSRGTFKDRSPYRTLMSVPIGEVPSPYNARFESVAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 246 - protein, 445 aa, synthetic construct
VIYDVRDDVLTNLTITLDDIKTDLTTLINTRVDKLEMMINDNVTTILASGATPRGSGGVILTGNSSLCPIRGWAPHSKDNSVRIGSKGDVFVIREPFISCSHLECRTFFLTQGALLNDKHSRGTFKDRSPYRTLMSVPIGEVPSPYNARFESVAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 247 - protein, 443 aa, synthetic construct
VIYDVRDDVLTNLTITLDDIKTDLTTLINTRVDKLEMMINDNVTTILASGATPRGGVILTGNSSLCPIRGWAPHSKDNSVRIGSKGDVFVIREPFISCSHLECRTFFLTQGALLNDKHSRGTFKDRSPYRTLMSVPIGEVPSPYNARFESVAWSASACHDGMGWLTIGITGPDDGAVAVLKYNGIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 248 - protein, 447 aa, synthetic construct
VIYDVRDDVLTNLTITLDDIKTDLTTLINTRVDKLEMMINDNVTTILASGSGKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 249 - protein, 445 aa, synthetic construct
VIYDVRDDVLTNLTITLDDIKTDLTTLINTRVDKLEMMINDNVTTILASGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 250 - protein, 447 aa, synthetic construct
VIYDVRDDVLTNLTITLDDIKTDLTTLINTRVDKLEMMINDNVTTILASGGGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 251 - protein, 443 aa, synthetic construct
VIYDVRDDVLTNLTITLDDIKTDLTTLINTRVDKLEMMINDNVTTILASGATPRGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 252 - protein, 436 aa, synthetic construct
NLTITLDDIKTDLTTLINTRVDKLEMMINDNVTTILASGSGKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 253 - protein, 434 aa, synthetic construct
NLTITLDDIKTDLTTLINTRVDKLEMMINDNVTTILASGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 254 - protein, 436 aa, synthetic construct
NLTITLDDIKTDLTTLINTRVDKLEMMINDNVTTILASGGGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 255 - protein, 432 aa, synthetic construct
NLTITLDDIKTDLTTLINTRVDKLEMMINDNVTTILASGATPRGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 256 - protein, 432 aa, synthetic construct
TLDDIKTDLTTLINTRVDKLEMMINDNVTTILASGSGKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 257 - protein, 430 aa, synthetic construct
TLDDIKTDLTTLINTRVDKLEMMINDNVTTILASGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 258 - protein, 432 aa, synthetic construct
TLDDIKTDLTTLINTRVDKLEMMINDNVTTILASGGGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 259 - protein, 428 aa, synthetic construct
TLDDIKTDLTTLINTRVDKLEMMINDNVTTILASGATPRGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 260 - protein, 447 aa, synthetic construct
VIYDVRDDVLTNLTITLDDIKTDLTTLINTRVDKLEMMINDNVTTILASGSGKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 261 - protein, 445 aa, synthetic construct
VIYDVRDDVLTNLTITLDDIKTDLTTLINTRVDKLEMMINDNVTTILASGATPRGSGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 262 - protein, 447 aa, synthetic construct
VIYDVRDDVLTNLTITLDDIKTDLTTLINTRVDKLEMMINDNVTTILASGGGATPRGSGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 263 - protein, 443 aa, synthetic construct
VIYDVRDDVLTNLTITLDDIKTDLTTLINTRVDKLEMMINDNVTTILASGATPRGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 264 - protein, 436 aa, synthetic construct
NLTITLDDIKTDLTTLINTRVDKLEMMINDNVTTILASGSGKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 265 - protein, 434 aa, synthetic construct
NLTITLDDIKTDLTTLINTRVDKLEMMINDNVTTILASGATPRGSGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 266 - protein, 436 aa, synthetic construct
NLTITLDDIKTDLTTLINTRVDKLEMMINDNVTTILASGGGATPRGSGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 267 - protein, 432 aa, synthetic construct
NLTITLDDIKTDLTTLINTRVDKLEMMINDNVTTILASGATPRGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 268 - protein, 432 aa, synthetic construct
TLDDIKTDLTTLINTRVDKLEMMINDNVTTILASGSGKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 269 - protein, 430 aa, synthetic construct
TLDDIKTDLTTLINTRVDKLEMMINDNVTTILASGATPRGSGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 270 - protein, 432 aa, synthetic construct
TLDDIKTDLTTLINTRVDKLEMMINDNVTTILASGGGATPRGSGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 271 - protein, 428 aa, synthetic construct
TLDDIKTDLTTLINTRVDKLEMMINDNVTTILASGATPRGGEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 272 - protein, 447 aa, synthetic construct
VIYDVRDDVLTNLTITLDDIKTDLTTLINTRVDKLEMMINDNVTTILASGSGKEICPKLAEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 273 - protein, 445 aa, synthetic construct
VIYDVRDDVLTNLTITLDDIKTDLTTLINTRVDKLEMMINDNVTTILASGATPRGSGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 274 - protein, 447 aa, synthetic construct
VIYDVRDDVLTNLTITLDDIKTDLTTLINTRVDKLEMMINDNVTTILASGGGATPRGSGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 275 - protein, 443 aa, synthetic construct
VIYDVRDDVLTNLTITLDDIKTDLTTLINTRVDKLEMMINDNVTTILASGATPRGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 276 - protein, 436 aa, synthetic construct
NLTITLDDIKTDLTTLINTRVDKLEMMINDNVTTILASGSGKEICPKLAEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 277 - protein, 434 aa, synthetic construct
NLTITLDDIKTDLTTLINTRVDKLEMMINDNVTTILASGATPRGSGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 278 - protein, 436 aa, synthetic construct
NLTITLDDIKTDLTTLINTRVDKLEMMINDNVTTILASGGGATPRGSGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 279 - protein, 432 aa, synthetic construct
NLTITLDDIKTDLTTLINTRVDKLEMMINDNVTTILASGATPRGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 280 - protein, 432 aa, synthetic construct
TLDDIKTDLTTLINTRVDKLEMMINDNVTTILASGSGKEICPKLAEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 281 - protein, 430 aa, synthetic construct
TLDDIKTDLTTLINTRVDKLEMMINDNVTTILASGATPRGSGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 282 - protein, 432 aa, synthetic construct
TLDDIKTDLTTLINTRVDKLEMMINDNVTTILASGGGATPRGSGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 283 - protein, 428 aa, synthetic construct
TLDDIKTDLTTLINTRVDKLEMMINDNVTTILASGATPRGGEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 284 - protein, 447 aa, synthetic construct
VIYDVRDDVLTNLTITLDDIKTDLTTLINTRVDKLEMMINDNVTTILASGSGKEICPKLVEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 285 - protein, 445 aa, synthetic construct
VIYDVRDDVLTNLTITLDDIKTDLTTLINTRVDKLEMMINDNVTTILASGATPRGSGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 286 - protein, 447 aa, synthetic construct
VIYDVRDDVLTNLTITLDDIKTDLTTLINTRVDKLEMMINDNVTTILASGGGATPRGSGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 287 - protein, 443 aa, synthetic construct
VIYDVRDDVLTNLTITLDDIKTDLTTLINTRVDKLEMMINDNVTTILASGATPRGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 288 - protein, 436 aa, synthetic construct
NLTITLDDIKTDLTTLINTRVDKLEMMINDNVTTILASGSGKEICPKLVEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 289 - protein, 434 aa, synthetic construct
NLTITLDDIKTDLTTLINTRVDKLEMMINDNVTTILASGATPRGSGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 290 - protein, 436 aa, synthetic construct
NLTITLDDIKTDLTTLINTRVDKLEMMINDNVTTILASGGGATPRGSGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 291 - protein, 432 aa, synthetic construct
NLTITLDDIKTDLTTLINTRVDKLEMMINDNVTTILASGATPRGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 292 - protein, 432 aa, synthetic construct
TLDDIKTDLTTLINTRVDKLEMMINDNVTTILASGSGKEICPKLVEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 293 - protein, 430 aa, synthetic construct
TLDDIKTDLTTLINTRVDKLEMMINDNVTTILASGATPRGSGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 294 - protein, 432 aa, synthetic construct
TLDDIKTDLTTLINTRVDKLEMMINDNVTTILASGGGATPRGSGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 295 - protein, 428 aa, synthetic construct
TLDDIKTDLTTLINTRVDKLEMMINDNVTTILASGATPRGGEYRNWSKPQCKITGFAPFSKDNSIRLSAGGDIWVTREPYVSCDPGKCYQFALGQGTTLDNKHSNDTIHDRTPYRTLLMNELGVPFHLGTRQVCIAWSSSSCHDGKAWLHVCITGYDKNATASFIYDGRLVDSIGSWSKNILRTQESECVCINGTCTVVMTDGSASERADTKILFIEEGKIVHISPLSGSAQHVEECSCYPRYPGVRCVCRDNWKGSNRPVVDINVKDYSIVSSYVCSGLVGDTPRKNDRSSSSYCRNPNNEKGNHGVKGWAFDDGNDVWMGRTISEESRSGYETFKVIGGWSTPNSKLQINRQVIVDSGNRSGYSGIFSVEGKSCINRCFYVELIRGREQETRVWWTSNSIVVFCGTSGTYGTGSWPDGADINLMPI

SEQ ID NO: 296 - protein, 447 aa, synthetic construct
VIYDVRDDVLTNLTITLDDIKTDLTTLINTRVDKLEMMINDNVTTILASGSGKEICPKVVEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 297 - protein, 445 aa, synthetic construct
VIYDVRDDVLTNLTITLDDIKTDLTTLINTRVDKLEMMINDNVTTILASGATPRGSGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 298 - protein, 447 aa, synthetic construct
VIYDVRDDVLTNLTITLDDIKTDLTTLINTRVDKLEMMINDNVTTILASGGGATPRGSGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 299 - protein, 443 aa, synthetic construct
VIYDVRDDVLTNLTITLDDIKTDLTTLINTRVDKLEMMINDNVTTILASGATPRGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 300 - protein, 436 aa, synthetic construct
NLTITLDDIKTDLTTLINTRVDKLEMMINDNVTTILASGSGKEICPKVVEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 301 - protein, 434 aa, synthetic construct
NLTITLDDIKTDLTTLINTRVDKLEMMINDNVTTILASGATPRGSGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 302 - protein, 436 aa, synthetic construct
NLTITLDDIKTDLTTLINTRVDKLEMMINDNVTTILASGGGATPRGSGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 303 - protein, 432 aa, synthetic construct
NLTITLDDIKTDLTTLINTRVDKLEMMINDNVTTILASGATPRGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 304 - protein, 432 aa, synthetic construct
TLDDIKTDLTTLINTRVDKLEMMINDNVTTILASGSGKEICPKVVEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 305 - protein, 430 aa, synthetic construct
TLDDIKTDLTTLINTRVDKLEMMINDNVTTILASGATPRGSGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 306 - protein, 432 aa, synthetic construct
TLDDIKTDLTTLINTRVDKLEMMINDNVTTILASGGGATPRGSGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 307 - protein, 428 aa, synthetic construct
TLDDIKTDLTTLINTRVDKLEMMINDNVTTILASGATPRGGEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPI

SEQ ID NO: 308 - protein, 451 aa, synthetic construct
VIYDVRDDVLTNLTITLDDIKTDLTTLINTRVDKLEMMINDNVTTILASGSGTKGATLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 309 - protein, 449 aa, synthetic construct
VIYDVRDDVLTNLTITLDDIKTDLTTLINTRVDKLEMMINDNVTTILASGATPRGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 310 - protein, 449 aa, synthetic construct
VIYDVRDDVLTNLTITLDDIKTDLTTLINTRVDKLEMMINDNVTTILASGGGSGGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 311 - protein, 447 aa, synthetic construct
VIYDVRDDVLTNLTITLDDIKTDLTTLINTRVDKLEMMINDNVTTILASGATPRGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 312 - protein, 440 aa, synthetic construct
NLTITLDDIKTDLTTLINTRVDKLEMMINDNVTTILASGSGTKGATLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 313 - protein, 438 aa, synthetic construct
NLTITLDDIKTDLTTLINTRVDKLEMMINDNVTTILASGATPRGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 314 - protein, 438 aa, synthetic construct
NLTITLDDIKTDLTTLINTRVDKLEMMINDNVTTILASGGGSGGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 315 - protein, 436 aa, synthetic construct
NLTITLDDIKTDLTTLINTRVDKLEMMINDNVTTILASGATPRGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 316 - protein, 436 aa, synthetic construct
TLDDIKTDLTTLINTRVDKLEMMINDNVTTILASGSGTKGATLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 317 - protein, 434 aa, synthetic construct
TLDDIKTDLTTLINTRVDKLEMMINDNVTTILASGATPRGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 318 - protein, 434 aa, synthetic construct
TLDDIKTDLTTLINTRVDKLEMMINDNVTTILASGGGSGGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 319 - protein, 432 aa, synthetic construct
TLDDIKTDLTTLINTRVDKLEMMINDNVTTILASGATPRGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 320 - protein, 451 aa, synthetic construct
VIYDVRDDVLTNLTITLDDIKTDLTTLINTRVDKLEMMINDNVTTILASGSGTKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 321 - protein, 449 aa, synthetic construct
VIYDVRDDVLTNLTITLDDIKTDLTTLINTRVDKLEMMINDNVTTILASGATPRGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 322 - protein, 449 aa, synthetic construct
VIYDVRDDVLTNLTITLDDIKTDLTTLINTRVDKLEMMINDNVTTILASGGGSGGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 323 - protein, 447 aa, synthetic construct
VIYDVRDDVLTNLTITLDDIKTDLTTLINTRVDKLEMMINDNVTTILASGATPRGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 324 - protein, 440 aa, synthetic construct
NLTITLDDIKTDLTTLINTRVDKLEMMINDNVTTILASGSGTKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 325 - protein, 438 aa, synthetic construct
NLTITLDDIKTDLTTLINTRVDKLEMMINDNVTTILASGATPRGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 326 - protein, 438 aa, synthetic construct
NLTITLDDIKTDLTTLINTRVDKLEMMINDNVTTILASGGGSGGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 327 - protein, 436 aa, synthetic construct
NLTITLDDIKTDLTTLINTRVDKLEMMINDNVTTILASGATPRGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 328 - protein, 436 aa, synthetic construct
TLDDIKTDLTTLINTRVDKLEMMINDNVTTILASGSGTKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 329 - protein, 434 aa, synthetic construct
TLDDIKTDLTTLINTRVDKLEMMINDNVTTILASGATPRGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 330 - protein, 434 aa, synthetic construct
TLDDIKTDLTTLINTRVDKLEMMINDNVTTILASGGGSGGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 331 - protein, 432 aa, synthetic construct
TLDDIKTDLTTLINTRVDKLEMMINDNVTTILASGATPRGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 332 - protein, 451 aa, synthetic construct
VIYDVRDDVLTNLTITLDDIKTDLTTLINTRVDKLEMMINDNVTTILASGSGTKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 333 - protein, 449 aa, synthetic construct
VIYDVRDDVLTNLTITLDDIKTDLTTLINTRVDKLEMMINDNVTTILASGATPRGSGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 334 - protein, 449 aa, synthetic construct
VIYDVRDDVLTNLTITLDDIKTDLTTLINTRVDKLEMMINDNVTTILASGGGSGGSGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 335 - protein, 447 aa, synthetic construct
VIYDVRDDVLTNLTITLDDIKTDLTTLINTRVDKLEMMINDNVTTILASGATPRGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 336 - protein, 440 aa, synthetic construct
NLTITLDDIKTDLTTLINTRVDKLEMMINDNVTTILASGSGTKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 337 - protein, 438 aa, synthetic construct
NLTITLDDIKTDLTTLINTRVDKLEMMINDNVTTILASGATPRGSGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 338 - protein, 438 aa, synthetic construct
NLTITLDDIKTDLTTLINTRVDKLEMMINDNVTTILASGGGSGGSGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 339 - protein, 436 aa, synthetic construct
NLTITLDDIKTDLTTLINTRVDKLEMMINDNVTTILASGATPRGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 340 - protein, 436 aa, synthetic construct
TLDDIKTDLTTLINTRVDKLEMMINDNVTTILASGSGTKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 341 - protein, 434 aa, synthetic construct
TLDDIKTDLTTLINTRVDKLEMMINDNVTTILASGATPRGSGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 342 - protein, 434 aa, synthetic construct
TLDDIKTDLTTLINTRVDKLEMMINDNVTTILASGGGSGGSGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 343 - protein, 432 aa, synthetic construct
TLDDIKTDLTTLINTRVDKLEMMINDNVTTILASGATPRGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 344 - protein, 451 aa, synthetic construct
VIYDVRDDVLTNLTITLDDIKTDLTTLINTRVDKLEMMINDNVTTILASGSGTKGMTLLLSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 345 - protein, 449 aa, synthetic construct
VIYDVRDDVLTNLTITLDDIKTDLTTLINTRVDKLEMMINDNVTTILASGATPRGSGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 346 - protein, 449 aa, synthetic construct
VIYDVRDDVLTNLTITLDDIKTDLTTLINTRVDKLEMMINDNVTTILASGGGSGGSGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 347 - protein, 447 aa, synthetic construct
VIYDVRDDVLTNLTITLDDIKTDLTTLINTRVDKLEMMINDNVTTILASGATPRGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 348 - protein, 440 aa, synthetic construct
NLTITLDDIKTDLTTLINTRVDKLEMMINDNVTTILASGSGTKGMTLLLSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 349 - protein, 438 aa, synthetic construct
NLTITLDDIKTDLTTLINTRVDKLEMMINDNVTTILASGATPRGSGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 350 - protein, 438 aa, synthetic construct
NLTITLDDIKTDLTTLINTRVDKLEMMINDNVTTILASGGGSGGSGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 351 - protein, 436 aa, synthetic construct
NLTITLDDIKTDLTTLINTRVDKLEMMINDNVTTILASGATPRGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 352 - protein, 436 aa, synthetic construct
TLDDIKTDLTTLINTRVDKLEMMINDNVTTILASGSGTKGMTLLLSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 353 - protein, 434 aa, synthetic construct
TLDDIKTDLTTLINTRVDKLEMMINDNVTTILASGATPRGSGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 354 - protein, 434 aa, synthetic construct
TLDDIKTDLTTLINTRVDKLEMMINDNVTTILASGGGSGGSGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 355 - protein, 432 aa, synthetic construct
TLDDIKTDLTTLINTRVDKLEMMINDNVTTILASGATPRGGSEPEWTYPRLSCQGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALIKIKYGEAYTDTYHSYANNILRTQESACNCIGGDCYLMITDGSASGISKCRFLKIREGRIIKEIFPTGRVEHTEECTCGFASNKTIECACRDNSYTAKRPFVKLNVETDTAEIRLMCTETYLDTPRPDDGSITGPCESNGDKGRGGIKGGFVHQRMASKIGRWYSRTMSKTERMGMELYVKYDGDPWTDSDALAPSGVMVSMKEPGWYSFGFEIKDKKCDVPCIGIEMVHDGGKKTWHSAATAIYCLMGSGQLLWDTVTGVDMAL

SEQ ID NO: 356 - protein, 451 aa, synthetic construct
VIYDVRDDVLTNLTITLDDIKTDLTTLINTRVDKLEMMINDNVTTILASGSGTKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 357 - protein, 449 aa, synthetic construct
VIYDVRDDVLTNLTITLDDIKTDLTTLINTRVDKLEMMINDNVTTILASGATPRGSGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 358 - protein, 449 aa, synthetic construct
VIYDVRDDVLTNLTITLDDIKTDLTTLINTRVDKLEMMINDNVTTILASGGGSGGSGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 359 - protein, 447 aa, synthetic construct
VIYDVRDDVLTNLTITLDDIKTDLTTLINTRVDKLEMMINDNVTTILASGATPRGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 360 - protein, 440 aa, synthetic construct
NLTITLDDIKTDLTTLINTRVDKLEMMINDNVTTILASGSGTKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 361 - protein, 438 aa, synthetic construct
NLTITLDDIKTDLTTLINTRVDKLEMMINDNVTTILASGATPRGSGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 362 - protein, 438 aa, synthetic construct
NLTITLDDIKTDLTTLINTRVDKLEMMINDNVTTILASGGGSGGSGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 363 - protein, 436 aa, synthetic construct
NLTITLDDIKTDLTTLINTRVDKLEMMINDNVTTILASGATPRGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 364 - protein, 436 aa, synthetic construct
TLDDIKTDLTTLINTRVDKLEMMINDNVTTILASGSGTKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 365 - protein, 434 aa, synthetic construct
TLDDIKTDLTTLINTRVDKLEMMINDNVTTILASGATPRGSGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 366 - protein, 434 aa, synthetic construct
TLDDIKTDLTTLINTRVDKLEMMINDNVTTILASGGGSGGSGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 367 - protein, 432 aa, synthetic construct
TLDDIKTDLTTLINTRVDKLEMMINDNVTTILASGATPRGGPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 368 - protein, 457 aa, synthetic construct
IINETADDIVYRLTVIIDDRYESLKNLITLRADRLEMIINDNVSTILASGSGTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 369 - protein, 457 aa, synthetic construct
IINETADDIVYRLTVIIDDRYESLKNLITLRADRLEMIINDNVSTILASGSGIVYLTNTTIEKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 370 - protein, 463 aa, synthetic construct
IINETADDIVYRLTVIIDDRYESLKNLITLRADRLEMIINDNVSTILASGSGNASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 371 - protein, 457 aa, synthetic construct
VIYDVRDDVLTNLTITLDDIKTDLTTLINTRVDKLEMMINDNVTTILASGSGTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 372 - protein, 438 aa, synthetic construct
SSSDYSDLQRVKQELLEEVKKELQKVKEEIIEAFVQELRKRGSLVPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 373 - protein, 438 aa, synthetic construct
SSSDYSDLQRVKQELLEEVKKELQKVKEEIIEAFVQELRKRGSLVPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 374 - protein, 442 aa, synthetic construct
SSSDYSDLQRVKQELLEEVKKELQKVKEEIIEAFVQELRKRGSLVPRGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 375 - protein, 452 aa, synthetic construct
IINETADDIVYRLTVIIDDRYESLKNLITLRADRLEMIINDNVSTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 376 - protein, 443 aa, synthetic construct
IINETADDIVYRLTVIIDDRYESLKNLITLRADRLEMIINDNVSTILASGSGSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 377 - protein, 445 aa, synthetic construct
IINETADDIVYRLTVIIDDRYESLKNLITLRADRLEMIINDNVSTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 378 - protein, 441 aa, synthetic construct
IINETADDIVYRLTVIIDDRYESLKNLITLRADRLEMIINDNVSTILASGGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 379 - protein, 436 aa, synthetic construct
IINETADDIVYRLTVIIDDRYESLKNLITLRADRLEMIINDNVSTILASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 380 - protein, 452 aa, synthetic construct; identical to SEQ ID NO: 56

SEQ ID NO: 381 - protein, 443 aa, synthetic construct
VIYDVRDDVLTNLTITLDDIKTDLTTLINTRVDKLEMMINDNVTTILASGSGSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 382 - protein, 445 aa, synthetic construct; identical to SEQ ID NO: 58

SEQ ID NO: 383 - protein, 441 aa, synthetic construct
VIYDVRDDVLTNLTITLDDIKTDLTTLINTRVDKLEMMINDNVTTILASGGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 384 - protein, 436 aa, synthetic construct
VIYDVRDDVLTNLTITLDDIKTDLTTLINTRVDKLEMMINDNVTTILASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 385 - protein, 452 aa, synthetic construct
IINETADDIVYRLTVIIDDRYESLKNLITLRADRLEMIINDNVSTILASGSGNTTIEKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 386 - protein, 443 aa, synthetic construct
IINETADDIVYRLTVIIDDRYESLKNLITLRADRLEMIINDNVSTILASGSGPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 387 - protein, 445 aa, synthetic construct
IINETADDIVYRLTVIIDDRYESLKNLITLRADRLEMIINDNVSTILASGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 388 - protein, 441 aa, synthetic construct
IINETADDIVYRLTVIIDDRYESLKNLITLRADRLEMIINDNVSTILASGGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 389 - protein, 436 aa, synthetic construct
IINETADDIVYRLTVIIDDRYESLKNLITLRADRLEMIINDNVSTILAAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 390 - protein, 455 aa, synthetic construct
IINETADDIVYRLTVIIDDRYESLKNLITLRADRLEMIINDNVSTILASGSGNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 391 - protein, 447 aa, synthetic construct
IINETADDIVYRLTVIIDDRYESLKNLITLRADRLEMIINDNVSTILASGSGTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 392 - protein, 449 aa, synthetic construct
IINETADDIVYRLTVIIDDRYESLKNLITLRADRLEMIINDNVSTILASGATPRGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 393 - protein, 445 aa, synthetic construct
IINETADDIVYRLTVIIDDRYESLKNLITLRADRLEMIINDNVSTILASGGSGGPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 394 - protein, 440 aa, synthetic construct
IINETADDIVYRLTVIIDDRYESLKNLITLRADRLEMIINDNVSTILALPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 395 - protein, 457 aa, synthetic construct
VINDTRDDILTNLTITLDDIKTDLTTLINTRADKLEMIINDNVTTILASGSGTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 396 - protein, 457 aa, synthetic construct
VINDTADDILTNLTITIDDIYTDLTTLINTRADKLEMIINDNVTTILASGSGTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 397 - protein, 457 aa, synthetic construct
VINDTRDDVLTNLTITLDDIKTNLTTLINTRVDKLEMMINDNVTTILASGSGTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 398 - protein, 457 aa, synthetic construct
VINDTRDDILTNLTITLDDIKTNLTTLINTRADKLEMIINDNVTTILASGSGTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 399 - protein, 443 aa, synthetic construct; identical to SEQ ID NO: 59

SEQ ID NO: 400 - protein, 447 aa, synthetic construct
VIYDVRDDVLTNLTITLDDIKTDLTTLINTRVDKLEMMINDNVTTILASGGGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 401 - protein, 457 aa, synthetic construct
VIYDVRDDVLTNLTITLDDIKTDLTTLINTRVDKLEMMINDNVTTILASGSGIVYLTNTTIEKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 402 - protein, 445 aa, synthetic construct; identical to SEQ ID NO: 249

SEQ ID NO: 403 - protein, 447 aa, synthetic construct; identical to SEQ ID NO: 250

SEQ ID NO: 404 - protein, 457 aa, synthetic construct
VINDTRDDVLTNLTITLDDIKTNLTTLINTRVDKLEMMINDNVTTILASGSGIVYLTNTTIEKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 405 - protein, 445 aa, synthetic construct
VINDTRDDVLTNLTITLDDIKTNLTTLINTRVDKLEMMINDNVTTILASGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 406 - protein, 447 aa, synthetic construct
VINDTRDDVLTNLTITLDDIKTNLTTLINTRVDKLEMMINDNVTTILASGGGATPRGSGGEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 407 - protein, 449 aa, synthetic construct; identical to SEQ ID NO: 321

SEQ ID NO: 408 - protein, 449 aa, synthetic construct; identical to SEQ ID NO: 322

SEQ ID NO: 409 - protein, 452 aa, synthetic construct; identical to SEQ ID NO: 68

SEQ ID NO: 410 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRQFFLTQGALLNDKHSNGTIKDRSPYRTLMSVPIGSVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 411 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRQFFLTQGALLNDKHSNGTIKDRSPYRTLMSVPIGSVPSPYNARFESIAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 412 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRQFFLTQGALLNDKHSNGTIKDRSPYRTLMSVPIGSVPSPYNARFESVAWSASACHDGINWLTIGITGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTTGWSWPDGAELPFTIDK

SEQ ID NO: 413 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPYSKDNSVRIGSKGDVFVIREPFISCSPLECRQFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 414 - protein, 452 aa, synthetic construct
VIYDVRDDVLTNLTITLDDIKTDLTTLINTRVDKLEMMINDNVTTILASGSGSNTNFAAGQSVVSVKLAGNSSLCPVSGWAPLSKDNSVRIGSKGDVFVIREPFISCSPLECRQFFLTQGALLNDKHSNGTIKDRSPYRTLMSVPIGSVPSPYNARFESIAWSASACHDGINWLTIGITGPDSGAVAILKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVMHPELTGLDCIVPCFWVELIRGRPEENTIWTSGSSISFCGVNSDTVGWSWPDGAELPFTIDK

SEQ ID NO: 415 - protein, 452 aa, synthetic construct; identical to SEQ ID NO: 72

SEQ ID NO: 416 - protein, 449 aa, synthetic construct
DVRDDVLTNLTITLDDIKTDLTTLINTRVDKLEMMINDNVTTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 417 - protein, 445 aa, synthetic construct
DVLTNLTITLDDIKTDLTTLINTRVDKLEMMINDNVTTILASGSGSNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 418 - protein, 441 aa, synthetic construct; identical to SEQ ID NO: 60

SEQ ID NO: 419 - protein, 437 aa, synthetic construct; identical to SEQ ID NO: 64

SEQ ID NO: 420 - protein, 449 aa, synthetic construct
IINETADDIVYRLTVIIDDRYESLKNLITLRADRLEMIINDNVSTILASGATPRGSGGPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 421 - protein, 449 aa, synthetic construct; identical to SEQ ID NO: 333

SEQ ID NO: 422 - protein, 449 aa, synthetic construct; identical to SEQ ID NO: 334

SEQ ID NO: 423 - protein, 387 aa, synthetic construct
VKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDN

SEQ ID NO: 424 - protein, 454 aa, synthetic construct
VIYDVRDDVLTNLTITLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 425 - protein, 449 aa, synthetic construct
VIYDVRDDVLTNLTITLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 426 - protein, 447 aa, synthetic construct
VIYDVRDDVLTNLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 427 - protein, 445 aa, synthetic construct
VIYDVRDDVLTNLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 428 - protein, 443 aa, synthetic construct
NLTITLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 429 - protein, 438 aa, synthetic construct
NLTITLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 430 - protein, 436 aa, synthetic construct
NLTITLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 431 - protein, 434 aa, synthetic construct
NLTITLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 432 - protein, 439 aa, synthetic construct
TLDDIKTDLTTLINTRVDKLEMMINDNVTTILASGSGSNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 433 - protein, 434 aa, synthetic construct
TLDDIKTDLTTLINTRVDKLEMMINDNVTTILASGSGAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 434 - protein, 432 aa, synthetic construct
TLDDIKTDLTTLINTRVDKLEMMINDNVTTILASGATPRGSGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 435 - protein, 430 aa, synthetic construct
TLDDIKTDLTTLINTRVDKLEMMINDNVTTILASGATPRGGVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 436 - protein, 454 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 437 - protein, 449 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 438 - protein, 447 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 439 - protein, 445 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 440 - protein, 454 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 441 - protein, 449 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 442 - protein, 447 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 443 - protein, 445 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGSVPSPYNARFESIAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 444 - protein, 454 aa, synthetic construct
VIYDVRDDVLTNLTITLDDIKTDLTTLINTRVDKLEMMINDNVTTILASGSGSNTNSAARQSVASVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 445 - protein, 449 aa, synthetic construct
VIYDVRDDVLTNLTITLDDIKTDLTTLINTRVDKLEMMINDNVTTILASGSGAARQSVASVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 446 - protein, 447 aa, synthetic construct
VIYDVRDDVLTNLTITLDDIKTDLTTLINTRVDKLEMMINDNVTTILASGATPRGSGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 447 - protein, 445 aa, synthetic construct
VIYDVRDDVLTNLTITLDDIKTDLTTLINTRVDKLEMMINDNVTTILASGATPRGGVKLAGNSSLCPVSGWAPYSKDNSVRIGSKGDVFVIREPFISCSPLECRTFFLTQGALLNDKHSNGTIKDRSPYRTLMSVPIGEVPSPYNARFESVAWSASACHDGTNWLTIGITGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDNEF

SEQ ID NO: 448 - protein, 748 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVSAGSGGSGSGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 449 - protein, 751 aa, synthetic construct
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVGGSAGSGGSGSGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 450 - protein, 762 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASAGSGGSGSGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 451 - protein, 764 aa, synthetic construct
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASAGSGGSGSGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 452 - protein, 522 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVSA

SEQ ID NO: 453 - protein, 525 aa, synthetic construct
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVGGSA

SEQ ID NO: 454 - protein, 536 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASA

SEQ ID NO: 455 - protein, 538 aa, synthetic construct
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASA

SEQ ID NO: 456 - protein, 11 aa, synthetic construct
GSGGSGSGSGS

SEQ ID NO: 457 - protein, 12 aa, synthetic construct
EKAAKAEEAARK

SEQ ID NO: 458 - protein, 202 aa, synthetic construct
EELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 459 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 460 - protein, 153 aa, synthetic construct
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 461 - protein, 156 aa, synthetic construct
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 462 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 463 - protein, 119 aa, synthetic construct
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 464 - protein, 748 aa, synthetic construct
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCFPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVSAGSGGSGSGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 465 - protein, 520 aa, synthetic construct
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCFPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGV

SEQ ID NO: 466 - protein, 42 aa, synthetic construct
SSSDYSDLQRVKQELLEEVKKELQKVKEEIIEAFVQELRKRG

SEQ ID NO: 467 - protein, 85 aa, synthetic construct
VELSSQQEYLKLKERYDALQRTQRNLLGEDLGPLSTKELESLERQLDSSLKQIRALRTQFMLDQSKERMLTETNKTLRLRLADGY

SEQ ID NO: 468 - protein, 113 aa, synthetic construct
ENTSSMKEMATLLTSLGVIQSAQEFESSRDASYVFARRALKSANYAEMTFNVCGLILSAEKSSARKVDENKQLLKIQESVESFRDIYKRFSEYQKEQNSLLMSNLSTLHIITD

SEQ ID NO: 469 - protein, 104 aa, synthetic construct
DKYIMPSDDFSNTFFPHDTDRLNYHADHLGDYDLETLCEESVLMGVINSIKLINLDMRLNHIEEQVKEIPKIINKLESIDRVLAKTNTALSTIEGHLVSMMIMI

SEQ ID NO: 470 - protein, 24 aa, synthetic construct
LLVQRMDAKLDLILALIGRLVRQS

SEQ ID NO: 471 - protein, 74 aa, synthetic construct
GDHYDDELFSDVQDIKTALAKIHEDNQKIISKLESLLLLKGEVESIKKQINRQNISISTLEGHLSSIMIAIPGL

SEQ ID NO: 472 - protein, 51 aa, synthetic construct
AVADVGDPFLLWKQQMDKWQNEYITDWQYHFEQYKKYQTYRHLDSDSCSGS

SEQ ID NO: 473 - protein, 31 aa, synthetic construct
MGILPSPGMPALLSLVSLLSVLLMGCVAETG

SEQ ID NO: 474 - protein, 25 aa, synthetic construct
MELLILKANAITTILTAVTFCFASG

SEQ ID NO: 475 - protein, 19 aa, synthetic construct
MSWKVMIIISLLITPQHGL

SEQ ID NO: 476 - protein, 17 aa, synthetic construct
MKAILVVLLYTFTTANA

SEQ ID NO: 477 - protein, 18 aa, synthetic construct
MPISILLIITTMIMASHC

SEQ ID NO: 478 - protein, 15 aa, synthetic construct
MFVFLVLLPLVSSQC

SEQ ID NO: 479 - protein, 19 aa, synthetic construct
MVPQVLLFVPLLGFSLCFG

SEQ ID NO: 480 - protein, 6 aa, synthetic construct
HHHHHH

SEQ ID NO: 481 - protein, 12 aa, synthetic construct
GSGGSGSGSGGS

SEQ ID NO: 482 - protein, 15 aa, synthetic construct
GSGGSGSGSGGSGSG

SEQ ID NO: 483 - protein, 8 aa, synthetic construct
GGSGGSGS

SEQ ID NO: 484 - protein, 8 aa, synthetic construct
GSGGSGSG

SEQ ID NO: 485 - protein, 7 aa, synthetic construct
GSGSGSG

SEQ ID NO: 486 - protein, 15 aa, synthetic construct
GSGSGSGSGSGSGSG

SEQ ID NO: 487 - skipped

SEQ ID NO: 488 - protein, 520 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGV

SEQ ID NO: 489 - protein, 751 aa, synthetic construct; identical to SEQ ID NO: 449

SEQ ID NO: 490 - protein, 521 aa, synthetic construct
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGV

SEQ ID NO: 491 - protein, 762 aa, synthetic construct; identical to SEQ ID NO: 450

SEQ ID NO: 492 - protein, 534 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAA

SEQ ID NO: 493 - protein, 764 aa, synthetic construct; identical to SEQ ID NO: 451

SEQ ID NO: 494 - protein, 536 aa, synthetic construct
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAA

SEQ ID NO: 495 - protein, 156 aa, synthetic construct
NQHSKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAAE